(19) United States
(12) Patent Application Publication
(10) Pub. No.: US 2026/0295033 A1
(43) Pub. Date: Oct. 1, 2026

(54) COMPOSITIONS OF MRNA-ENCODED VIRUS-LIKE PARTICLES, COMPOSITIONS OF MRNA-LIPID NANOPARTICLES, AND METHODS FOR USE THEREOF

(71) Applicant: VARIATION BIOTECHNOLOGIES INC., Ottawa (CA)

(72) Inventors: David Evander ANDERSON, Newton, MA (US); Tanvir AHMED, Manotick (CA); Rohan Aminbhai LALANI, Nepean (CA)

(21) Appl. No.: 19/479,546

(22) PCT Filed: Apr. 25, 2024
(86) PCT No.: PCT/CA2024/050544

§ 371 Date: Oct. 28, 2025

Related U.S. Application Data
(60) Provisional application No. 63/505,800, filed on Jun. 2, 2023.
(60) Provisional application No. 63/620,595, filed on Jan. 12, 2024.

(51) Int. Cl. A61K 39/215; A61K 39/00; A61K 39/245; A61P 37/04; C12N 7/00

(52) U.S. Cl. CPC A61K 39/215; A61K 39/245; A61P 37/04; C12N 7/00; A61K 2039/5258; A61K 2039/53; A61K 2039/55555; C12N 2710/16222; C12N 2710/16223; C12N 2710/16234; C12N 2770/20022; C12N 2770/20023; C12N 2770/20034

(57) ABSTRACT

An immunogenic composition is described, comprising mRNA encoding polypeptides constituting a virus-like particle (VLP), and a pharmaceutically acceptable carrier. The polypeptides constituting the VLP comprise: a first polypeptide comprising a gag protein found in murine leukemia virus (MEV) having at least 95% identity with the amino acid sequence of SEQ ID NO: 1; and at least one additional polypeptide, which may comprise a protein of a virus, such as EB V, CMV, or coronavirus, for example a spike glycoprotein from a beta coronavirus such as SARS-COV-2, SARS-COV-1 or MERS-COV; or the at least one additional polypeptide may comprise a protein expressed by a cancer cell or tumour cell. A lipid nanoparticle composition is also described, which may comprise the VLPs. Methods of treatment or prevention of infection, such as from EBV, CMV, or coronavirus, or for treatment or prevention of cancer are described.

### CROSS REFERENCE TO RELATED APPLICATIONS

This application claims the benefit of priority from U.S. Provisional Application No. 63/505,800 filed Jun. 2, 2023 and entitled “COMPOSITIONS OF MRNA-ENCODED VLPS AND METHODS FOR USE THEREOF” and U.S. Provisional Application No. 63/620,595 filed Jan. 12, 2024 and entitled “COMPOSITIONS OF MRNA-ENCODED VLPS, COMPOSITIONS OF MRNA-LIPID NANOPARTICLES, AND METHODS FOR USE THEREOF”, the contents of which are expressly incorporated by reference for all purposes.

### FIELD

The present disclosure relates generally to vaccines, in particular mRNA vaccines encoding virus-like particle vaccines for infectious disease and cancer. The present disclosure also relates generally to mRNA-lipid nanoparticle (LNP) formulations.

### BACKGROUND

Neutralizing antibodies are a correlate of protection against a wide array of viruses, including Epstein-Barr virus (EBV), cytomegalovirus (CMV), as well as those elicited from recently approved mRNA-based vaccines against SARS-COV-2 (Plotkin S A et al., 2010; Gilbert et al., 2022). Whereas induction of durable neutralizing antibody responses is of primary importance for prophylactic vaccines, induction of durable T cell immunity is of primary importance in the treatment of chronic viral infections and cancers. In addition to the magnitude and breadth of T cell responses induced by a vaccine, induction of polyfunctional T cells secreting multiple cytokines (e.g. IFN-γ, TNF-α) is an important component of vaccine efficacy (Panagioti E et al., 2018; Ramesh P et al., 2021) against both viral infections and cancers.

Based on analysis of individuals infected with SARS-COV-2 or vaccinated with approved mRNA-based vaccines, vaccination induces higher initial neutralizing antibody titers relative to naturally acquired immunity, but those antibody titers induced by mRNA-based vaccines decline more rapidly (Sette and Crotty, 2022). Conversely, infection with SARS-COV-2 induces higher frequencies of virus-specific CD4+ and CD8+ T cells than does vaccination.

A wide array of approaches have been described to produce virus-like particles (VLPs), which mimic the size structure of the relevant virus but lack genetic materials and are therefore non-infectious; they can be broadly classified into enveloped (eVLPs) or non-enveloped VLPs (Nooraei S et al., 2021). The presence of the lipid bilayer in eVLPs provides a natural site for glycoproteins to be anchored and adopt their preferred conformations, typically leading to enhanced immunity relative to a recombinant glycoprotein or chemical or genetic expression of the glycoprotein or an epitope from it on the surface of a non-enveloped VLP (Yan D et al., 2015).

Efforts to use mRNA to encode and produce VLPs in vivo have recently been reported and have demonstrated that mRNA-produced VLPs increased neutralizing antibody responses compared to mRNA expression of the antigens alone (Lu J et al., 2020; Hoffmann M A G et al., 2023). However, those studies failed to demonstrate the ability of mRNA-encoded VLPs to enhance T cell responses relative to mRNA expression of the antigen alone, and did not evaluate the potential impact on antibody durability.

Accordingly, a need exists to improve both the durability of antibody responses induced with mRNA-based vaccines and to increase the frequency of CD4+ and CD8+ T cells, particularly polyfunctional T cells.

### SUMMARY

The present disclosure provides methods and compositions useful for prophylaxis and/or treatment of human infectious diseases and cancer.

There is provided herein an immunogenic composition comprising mRNA encoding polypeptides that constitute a virus-like particle (VLP), and a pharmaceutically acceptable carrier, wherein the polypeptides that constitute the VLP comprise: a first polypeptide comprising a gag protein found in murine leukemia virus (MLV) having at least 95% identity with the amino acid sequence of SEQ ID NO:1; and at least one additional polypeptide comprising a protein of a virus against which an immunogenic effect is directed.

In an embodiment of the immunogenic composition, the at least one additional polypeptide comprises a protein of Epstein-Barr virus (EBV) or cytomegalovirus (CMV).

In another embodiment of the immunogenic composition, the at least one additional polypeptide may comprise a spike glycoprotein from a beta coronavirus. For example, the spike glycoprotein may be from SARS-COV-2, SARS-COV or MERS-COV. Such a spike glycoprotein may be a wild type protein, or spike glycoprotein may be a modified protein. According to an embodiment of the composition, the at least one additional polypeptide may comprise two spike glycoproteins. Further, the at least one additional polypeptide may comprise three spike glycoproteins. According to embodiments described herein, the modified spike glycoprotein may comprise such modifications as: a deletion at a furin cleavage site, or one or more lysine and/or valine residues is replaced with a proline residue, among others. A wide variety of modifications are envisioned.

The immunogenic composition may further comprise an adjuvant. For example, the adjuvant may be selected from the group consisting of cytokines, gel-type adjuvants, microbial adjuvants, oil-emulsion and emulsifier-based adjuvants, particulate adjuvants, synthetic adjuvants, polymer adjuvants, and combinations thereof. If a particulate adjuvant is to be employed, such examples of this include an aluminum salt.

The immunogenic composition may be formulated as lipid nanoparticles (LNPs) loaded with the mRNA. Other formulation types are envisioned, as described further herein.

The immunogenic composition may comprise two or more mRNAs encoding the polypeptides constituting the VLP, or may comprise one mRNA that encodes two or more of the polypeptides constituting the VLP.

Described herein is a method of treatment or prophylaxis of a subject having or at risk for a viral infection, comprising administering to the subject the immunogenic composition as described herein.

According to a further embodiment, there is described herein an immunogenic composition comprising mRNA encoding polypeptides that constitute a virus-like particle (VLP), and a pharmaceutically acceptable carrier. In this embodiment, the polypeptides that constitute the VLP comprise: a first polypeptide comprising a gag protein found in murine leukemia virus (MLV) having at least 95% identity with the amino acid sequence of SEQ ID NO: 1; and at least one additional polypeptide comprising a protein expressed by a tumor cell or cancer cell against which an immunogenic effect is directed.

A method of treatment or prophylaxis of a subject against a tumor or a cancer is also described, comprising administering to the subject this immunogenic composition, wherein the at least one additional polypeptide comprises a protein expressed by the tumor cell or cancer cell against which the immunogenic effect is directed.

In a further embodiment, there is described a method of producing a virus-like particle (VLP) by co-transfecting a host cell with a first vector comprising a nucleotide sequence encoding SEQ ID NO: 1 and a second vector comprising a nucleotide sequence of one or more of SEQ ID NOs: 6, 7, 10, 11, 14, 15, 18, 19, 22, 23, 27 or 31; and cultivating the host cell under conditions suitable for expression of the proteins encoded by the vectors.

A lipid nanoparticle composition is described herein, comprising C12-200, DOPE, cholesterol, and PEG-2000 DMG in a molar ratio of 33.5±1.7:19±1:45.1±2.3:2.4±0.1, optionally wherein the lipids are present at a concentration of about 2 to about 6 mg/mL, for example about 3 mg/mL to about 5 mg/mL, such as about 4 mg/mL. An embodiment of such a lipid nanoparticle further comprises phosphate at a concentration of about 8 mM to about 12 mM, such as about 10 mM; and sucrose, trehalose, or a combination thereof at a concentration of about 7 wt % to about 9 wt %, such as about 8 wt %. The lipid nanoparticle composition may be at a pH of about 7.0 to about 7.4, such as a pH of about 7.2.

The lipid nanoparticle composition may further comprising the immunogenic composition as defined herein. For example, the lipid nanoparticle composition may comprise mRNA, wherein the mRNA encodes polypeptides that constitute a virus-like particle (VLP), and the polypeptides that constitute the VLP comprise: a first polypeptide comprising a gag protein found in murine leukemia virus (MLV) having at least 95% identity with the amino acid sequence of SEQ ID NO:1; and at least one additional polypeptide comprising a protein of a virus against which an immunogenic effect is directed.

For example, the at least one additional polypeptide may comprise a protein of Epstein-Barr virus (EBV) or cytomegalovirus (CMV), or may comprise a spike glycoprotein from a beta coronavirus.

According to a further embodiment, the lipid nanoparticle composition may further comprise mRNA, wherein the mRNA encodes polypeptides that constitute a virus-like particle (VLP), and the polypeptides that constitute the VLP comprise: a first polypeptide comprising a gag protein found in murine leukemia virus (MLV) having at least 95% identity with the amino acid sequence of SEQ ID NO:1; and at least one additional polypeptide comprising a protein expressed by a tumor cell or cancer cell against which an immunogenic effect is directed.

A method of treatment or prophylaxis is described, for a subject having or at risk for a viral infection, comprising administering to the lipid nanoparticle composition described herein, such as may be directed to Epstein-Barr virus (EBV), cytomegalovirus (CMV), or coronavirus infection.

Further, a method of treatment or prophylaxis of a subject against a tumor or a cancer, may comprise administering to the subject the lipid nanoparticle composition described herein as directed to a cancer or tumor cell.

As used herein, the term “VLP” encompasses virus-like particles generally, whether enveloped (eVLP) or not.

In further embodiments, there is provided mRNA encoding enveloped virus-like particles (eVLPs) which comprise one or more Moloney Murine leukemia virus (MMLV) core proteins and include one or more heterologous proteins, such as for example, the MERS Spike protein, the Epstein-Barr virus (EBV) envelope glycoprotein gp350 and/or EBV nuclear antigen 1 (EBNA1) and/or EBV glycoprotein gB, or the Cytomegalovirus (CMV) envelope glycoprotein gB.

The present disclosure also encompasses a combination of antigens (e.g., envelope glycoproteins and structural proteins) that leads to improved induction of beneficial immune responses in vivo through delivery of mRNA encoding eVLPs, for example that include induction of both humoral responses (e.g., production of neutralizing antibodies) and cellular responses (e.g., polyfunctional CD4+ and CD8+ T cells). eVLPs generated in vivo with exogenous mRNA are also characterized in that they contain no viral DNA and are non-infectious yet provide superior immune responses compared to biologically produced eVLPs or mRNA encoding the relevant antigens alone.

In some embodiments, the mRNA generated eVLPs are surrounded by a lipid membrane, optionally containing one or more epitopes from viral envelope glycoproteins (e.g., EBV gp350, MERS Spike glycoprotein, CMV gB glycoprotein) which are antigens that play a role in induction of virus-neutralizing antibodies.

In some embodiments, the provided mRNA compositions and methods to produce in vivo eVLPs contain one or more epitopes from viral structural proteins which are antigens that play a role in induction of cellular immune responses (e.g., T-cell responses). In some embodiments, utilized viral structural proteins both stimulate formation of CD4+T helper cells and induce CD8+ cytotoxic T lymphocytes (CTL) against EBV, MERS, or CMV.

In some embodiments described herein, mRNA vaccines and in vivo produced eVLP variants of viral envelope glycoproteins or structural proteins (e.g., variants of EBV gp350 and/or EBNA1 or CMV gB) are described. In some embodiments, a variant viral envelope glycoprotein is or comprises a fusion protein. In some embodiments, a variant of a viral glycoprotein comprises a heterologous protein domain (e.g., a transmembrane and/or cytoplasmic domain from a different protein). In some embodiments, a variant of a viral structural protein comprises a heterologous antigen or epitope. In some embodiments, eVLPs comprising variants of viral structural proteins are provided. In some embodiments, a variant of a viral structural protein is or comprises a fusion protein.

In some embodiments, the present disclosure provides mRNAs encoding the MMLV Gag structural protein and mRNA encoding a heterologous envelope glycoprotein. In some embodiments the polypeptide is from MERS-COV. In some embodiments the polypeptide is from EBV. In other embodiments the polypeptide is from CMV.

In some embodiments, the polypeptide represents a modified envelope glycoprotein from MERS-COV. In other embodiments the modified envelope glycoprotein is from EBV or CMV. In some embodiments modified envelope glycoproteins have been modified such that the transmembrane domain is replaced with the transmembrane domain of another virus. In a particularly preferred embodiment, the modified envelope glycoprotein is comprised of an extracellular domain from MERS-COV, EBV, or CMV, the transmembrane domain and cytoplasmic tail of which proteins have been replaced with the transmembrane domain and cytoplasmic tail from vesicular stomatitis virus (VSV) G protein. CMV gB contains the TMCyt from VSV-G. MERS S and EBV gp350 are full length proteins.

The present disclosure also provides lipid nanoparticle (LNP) compositions comprising: 1,1′-((2-(4-(2-((2-(bis(2-hydroxydodecyl)amino)ethyl) (2-hydroxydodecyl)amino)ethyl) piperazin-1-yl)ethyl)azanediyl)bis(dodecan-2-ol) (C12-200); 1,2-dioleoyl-sn-glycero-3-phosphoethanolamine (DOPE); cholesterol; and 1,2-dimyristoyl-rac-glycero-3-methoxypolyethylene glycol-2000 (PEG-2000 DMG). The formula of C12-200 is illustrated below:

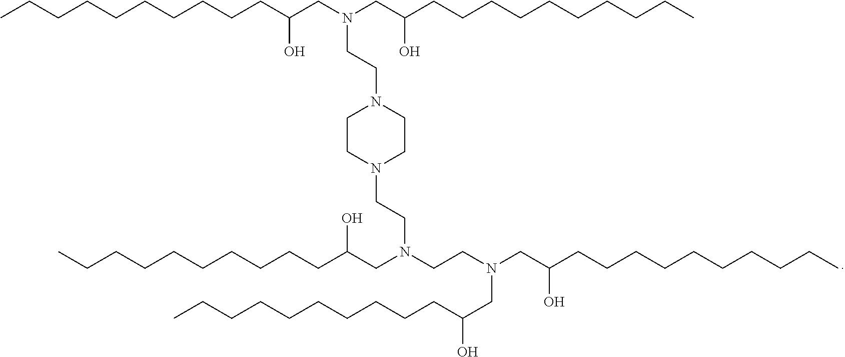 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

The C12-200, DOPE, cholesterol, and PEG-2000 DMG may be present in a molar ratio of 33.5±1.7:19±1:45.1±2.3:2.4±0.1. The lipids may be present in the composition at a concentration of about 2 to about 6 mg/mL, for example from about 3 to about 5 mg/mL, such as about 4 mg/mL.

The composition may be a buffered solution that includes phosphate, and sucrose and/or trehalose. For example, the composition may include about 8 mM to about 12 mM, such as about 10 mM, of phosphate; and/or the composition may include about 7 wt % to about 9 wt %, such as about 8 wt %, of sucrose, trehalose, or a combination thereof. The combination of phosphate and sucrose and/or trehalose may be used to maintain an osmolality and pH within physiological ranges, such as an osmolality from 250 to 350 mmol/kg and a pH from 7.0 to 7.4.

The composition may be at a pH of about 7.0 to about 7.4, such as a pH of about 7.2.

In a particular example, the lipid nanoparticle composition includes: C12:200, DOPE, cholesterol, and PEG2000-DMG in a molar ratio of 33.5:19:45.1:2.4, 10 mM of phosphate, and 8 wt % sucrose. In this particular example, the lipids are at a lipid concentration of 4 mg/mL and the composition is at a pH of 7.2.

LNPs according to the present disclosure may be used to encapsulate mRNA, such as the mRNA as described herein. The mRNA may be loaded into the LNPs using a citrate buffer, such as a 50 mM citrate buffer. The mRNA may be provided at a concentration from about 100 to about 300 μg/mL, such as at a concentration from about 175 to about 225 μg/mL, for example a concentration of about 200 μg/mL.

In some examples, the mRNA encodes polypeptides that constitute a virus-like particle (VLP). In one example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO:1; and at least one additional polypeptide comprising a spike glycoprotein from a beta coronavirus. In another example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO: 1; and at least one additional polypeptide comprising a protein expressed by a tumor cell or cancer cell against which an immunogenic effect is directed. In yet another example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO: 1; and at least one additional polypeptide comprising a protein of a virus against which an immunogenic effect is directed, such as a protein of Epstein-Barr virus (EBV) or cytomegalovirus (CMV).

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 is a negative stain transmission electron microscopic (nsTEM) image of eVLP particles produced in vitro after addition of an LNP formulation containing mRNA encoding for MMLV Gag.

FIG. 2 is a flow diagram depicting an mRNA-LNP transfection process.

FIG. 3 is a process flow diagram detailing cell culture procedures.

### DETAILED DESCRIPTION

Retroviruses are enveloped RNA viruses that belong to the family Retroviridae. After infection of a host cell by a retrovirus, RNA is transcribed into DNA via the enzyme reverse transcriptase. DNA is then incorporated into the host cell's genome by an integrase enzyme and thereafter replicates as part of the host cell's DNA. The Retroviridae family includes the following genus Alpharetrovirus, Betaretrovirus, Gammearetrovirus, Deltaretrovirus, Epsilonretrovirus, Lentivirus and Spumavirus. The hosts for this family of retroviruses generally are vertebrates. Retroviruses produce an infectious virion containing a spherical nucleocapsid (the viral genome in complex with viral structural proteins) surrounded by a lipid bilayer derived from the host cell membrane.

Retroviral vectors can be used to generate enveloped virions that are infectious and either replication-competent or replication-defective. Replication-competent infectious retroviral vectors contain all the necessary genes for virion synthesis and continue to propagate themselves once infection of the host cell occurs. Replication-defective infectious retroviral vectors do not spread after the initial infection. This is accomplished by replacement of most of the coding regions of the retrovirus with genes or nucleotide sequences to be transferred; so that the vector is incapable of making proteins required for additional rounds of replication.

Alternatively or additionally, retroviral vectors can be used to generate virus-like particles (VLPs) that lack a retrovirus-derived genome and are both non-infectious and non-replicating. Because of VLPs advantageous properties, VLPs may be utilized as an antigen delivery system. Furthermore, because VLPs are non-infectious, they can be administered safely as an immunogenic composition (e.g., a vaccine). VLPs are generally structurally similar to enveloped virions described above, but lack a retrovirus-derived genome, making it unlikely that viral replication will occur. Expression of capsid proteins (e.g., Gag) of some viruses (e.g., murine leukemia viruses, such as Moloney Murine leukemia virus (MMLV)) leads to self-assembly into particles similar to the corresponding native virus, which particles are free of viral genetic material.

A wide variety of VLPs have been prepared. For example, VLPs including single or multiple capsid proteins either with or without envelope proteins and/or surface glycoproteins have been prepared. In some cases, VLPs are non-enveloped and assemble by expression of just one major capsid protein, as shown for VLPs prepared from hepadnaviruses (e.g., Engerix™, GSK and Recombivax HB™, Merck), papillomaviruses (e.g., Cervarix™, GSK and Gardasil™, Merck), paroviruses, or polyomaviruses. In some embodiments, VLPs are enveloped and can comprise multiple antigenic proteins found in the corresponding native virus. VLPs typically resemble their corresponding native virus and can be multivalent particulate structures. In some embodiments, antigenic proteins may be presented internally within the VLP, as a component of the VLP structure, and/or on the surface of the VLP. The present disclosure encompasses the recognition that presentation of an antigen in the context of a VLP is advantageous for induction of neutralizing antibodies against the antigen as compared to other forms of antigen presentation, e.g., soluble antigens not associated with a VLP. Neutralizing antibodies most often recognize tertiary or quaternary structures; this often requires presenting antigenic proteins, like envelope glycoproteins, in their native viral conformation. Alternatively or additionally, VLPs may be useful for presenting antigens in a context which induces cellular immunity (e.g., T cell response). The present disclosure further encompasses the insight that use of antigen combinations in VLP systems can generate improved immune response.

Some embodiments utilize VLPs comprised of one or more retroviral structural proteins (e.g., Gag). In some embodiments, a structural protein for use as described herein is Alpharetrovirus (e.g., Avian Leukosis Virus), Betaretrovirus (Mouse Mammary Tumor Virus), Gammaretrovirus (Murine Leukemia Virus), Deltaretrovirus (Bovine Leukemia Virus), Epsilonretrovirus (Walley Dermal Sarcoma Virus), Lentivirus (Human Immunodeficiency Virus 1) or Spumavirus (Chimpanzee Foamy Virus) structural protein. In certain embodiments, a structural polyprotein is a Murine Leukemia Virus (MLV) structural protein. Genomes of these retroviruses are readily available in databases. The Gag genes of all these retroviruses have an overall structural similarity and within each group of retroviruses are conserved at the amino acid level. Retroviral Gag proteins primarily function in viral assembly. The Gag gene in the form of a polyprotein gives rise to the core structural proteins of the VLP. The MLV Gag gene encodes a 65 kDa polyprotein precursor which is proteolytically cleaved into 4 structural proteins (Matrix (MA); p12; Capsid (CA); and Nucleocapsid (NC)), by MLV protease, in the mature virion. Retroviruses assemble immature capsid composed of the Gag polyprotein formed from the Gag polypeptide but devoid of other viral elements like viral protease with Gag as the structural protein of the immature virus particle. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. The form of the Gag protein that mediates viral particle assembly is the polyprotein.

A retroviral structural protein for use in accordance with some described embodiments is a Gag polypeptide. As used herein, the term “Gag polypeptide” is the retrovirus derived structural polypeptide that is responsible for formation of the VLPs described herein and refers to a polypeptide sequence whose amino acid sequence includes at least one characteristic sequence of Gag. A wide variety of Gag sequences from various retroviruses are known in the art and those of ordinary skill in the art, referring to such sequences, can readily identify sequences that are characteristic of Gag proteins generally, and/or of particular Gag polypeptides.

In some embodiments, a suitable Gag polypeptide is substantially identical and/or substantially homologous to a known retroviral Gag polypeptide. For example, a Gag polypeptide may be a modified retroviral Gag polypeptide containing one or more amino acid substitutions, deletions, and/or insertions as compared to a wild-type or naturally-occurring Gag polypeptide (e.g., SEQ ID NO:1), while retaining substantial self-assembly activity. Thus, in some embodiments, a Gag polypeptide suitable for uses described herein is substantially identical to and/or substantially homologous to an MMLV Gag polypeptide (SEQ ID NO:1). In some embodiments, a suitable Gag polypeptide has an amino acid sequence of at least 50%, 55%, 60%, 65%, 70%, 75%, 80%, 85%, 90%, 91%, 92%, 93%, 94%, 95%, 96%, 97%, 98%, 99% sequence identity to SEQ ID NO:1. In some embodiments, a Gag polypeptide suitable for use herein is substantially identical to an MMLV Gag polypeptide (SEQ ID NO:1).

SEQ ID NO: 1 is an MMLV-Gag Amino Acid Sequence
MGQTVTTPLSLTLGHWKDVERIAHNQSVDVKKRRWVTFCSAEWPTENVGWPRDGTENRDLITQVKIKV
FSPGPHGHPDQVPYIVTWEALAFDPPPWVKPFVHPKPPPPLPPSAPSLPLEPPRSTPPRSSLYPALTP
SLGAKPKPQVLSDSGGPLIDLLTEDPPPYRDPRPPPSDRDGNGGEATPAGEAPDPSPMASRLRGRREP
PVADSTTSQAFPLRAGGNGQLQYWPFSSSDLYNWKNNNPSFSEDPGKLTALIESVLITHQPTWDDCQQ
LLGTLLTGEEKQRVLLEARKAVRGDDGRPTQLPNEVDAAFPLERPDWDYTTQAGRNHLVHYRQLLLAG
LQNAGRSPTNLAKVKGITQGPNESPSAFLERLKEAYRRYTPYDPEDPGQETNVSMSFIWQSAPDIGRK
LERLEDLKNKTLGDLVREAEKIENKRETPEEREERIRRETEEKEERRRTEDEQKEKERDRRRHREMSK
LLATVVSGQKQDRQGGERRRSQLDRDQCAYCKEKGHWAKDCPKKPRGPRGPRPQTSLLTLDD
SEQ ID NO: 2 is MMLV-Gag Nucleotide Sequence
ATGGGCCAGACTGTTACCACTCCCTTAAGTTTGACCTTAGGTCACTGGAAAGATGTCGAGCGGATCGC
TCACAACCAGTCGGTAGATGTCAAGAAGAGACGTTGGGTTACCTTCTGCTCTGCAGAATGGCCAACCT
TTAACGTCGGATGGCCGCGAGACGGCACCTTTAACCGAGACCTCATCACCCAGGTTAAGATCAAGGTC
TTTTCACCTGGCCCGCATGGACACCCAGACCAGGTCCCCTACATCGTGACCTGGGAAGCCTTGGCTTT
TGACCCCCCTCCCTGGGTCAAGCCCTTTGTACACCCTAAGCCTCCGCCTCCTCTTCCTCCATCCGCCC
CGTCTCTCCCCCTTGAACCTCCTCGTTCGACCCCGCCTCGATCCTCCCTTTATCCAGCCCTCACTCCT
TCTCTAGGCGCCAAACCTAAACCTCAAGTTCTTTCTGACAGTGGGGGGCCGCTCATCGACCTACTTAC
AGAAGACCCCCCGCCTTATAGGGACCCAAGACCACCCCCTTCCGACAGGGACGGAAATGGTGGAGAAG
CGACCCCTGCGGGAGAGGCACCGGACCCCTCCCCAATGGCATCTCGCCTACGTGGGAGACGGGAGCCC
CCTGTGGCCGACTCCACTACCTCGCAGGCATTCCCCCTCCGCGCAGGAGGAAACGGACAGCTTCAATA
CTGGCCGTTCTCCTCTTCTGACCTTTACAACTGGAAAAATAATAACCCTTCTTTTTCTGAAGATCCAG
GTAAACTGACAGCTCTGATCGAGTCTGTTCTCATCACCCATCAGCCCACCTGGGACGACTGTCAGCAG
CTGTTGGGGACTCTGCTGACCGGAGAAGAAAAACAACGGGTGCTCTTAGAGGCTAGAAAGGCGGTGCG
GGGCGATGATGGGCGCCCCACTCAACTGCCCAATGAAGTCGATGCCGCTTTTCCCCTCGAGCGCCCAG
ACTGGGATTACACCACCCAGGCAGGTAGGAACCACCTAGTCCACTATCGCCAGTTGCTCCTAGCGGGT
CTCCAAAACGCGGGCAGAAGCCCCACCAATTTGGCCAAGGTAAAAGGAATAACACAAGGGCCCAATGA
GTCTCCCTCGGCCTTCCTAGAGAGACTTAAGGAAGCCTATCGCAGGTACACTCCTTATGACCCTGAGG
ACCCAGGGCAAGAAACTAATGTGTCTATGTCTTTCATTTGGCAGTCTGCCCCAGACATTGGGAGAAAG
TTAGAGAGGTTAGAAGATTTAAAAAACAAGACGCTTGGAGATTTGGTTAGAGAGGCAGAAAAGATCTT
TAATAAACGAGAAACCCCGGAAGAAAGAGAGGAACGTATCAGGAGAGAAACAGAGGAAAAAGAAGAAC
GCCGTAGGACAGAGGATGAGCAGAAAGAGAAAGAAAGAGATCGTAGGAGACATAGAGAGATGAGCAAG
CTATTGGCCACTGTCGTTAGTGGACAGAAACAGGATAGACAGGGAGGAGAACGAAGGAGGTCCCAACT
CGATCGCGACCAGTGTGCCTACTGCAAAGAAAAGGGGCACTGGGCTAAAGATTGTCCCAAGAAACCAC
GAGGACCTCGGGGACCAAGACCCCAGACCTCCCTCCTGACCCTAGATGACTAA
SEQ ID NO: 3 is a Codon Optimized MMLV-Gag Nucleotide Sequence
ATGGGACAGACCGTCACAACACCCCTGAGCCTGACCCTGGGACATTGGAAAGACGTGGAGAGGATCGC
ACATAACCAGAGCGTGGACGTGAAGAAACGGAGATGGGTCACATTCTGCAGTGCTGAGTGGCCAACTT
TTAATGTGGGATGGCCCCGAGACGGCACTTTCAACAGGGATCTGATCACCCAGGTGAAGATCAAGGTC
TTTAGCCCAGGACCTCACGGACATCCAGACCAGGTGCCTTATATCGTCACCTGGGAGGCACTGGCCTT
CGATCCCCCTCCATGGGTGAAGCCATTTGTCCACCCAAAACCACCTCCACCACTGCCTCCAAGTGCCC
CTTCACTGCCACTGGAACCACCCCGGAGCACACCACCCCGCAGCTCCCTGTATCCTGCTCTGACTCCA
TCTCTGGGCGCAAAGCCAAAACCACAGGTGCTGAGCGACTCCGGAGGACCACTGATTGACCTGCTGAC
AGAGGACCCCCCACCATACCGAGATCCTCGGCCTCCACCAAGCGACCGCGATGGAAATGGAGGAGAGG
CTACTCCTGCCGGCGAAGCCCCTGACCCATCTCCAATGGCTAGTAGGCTGCGCGGCAGGCGCGAGCCT
CCAGTGGCAGATAGCACCACATCCCAGGCCTTCCCTCTGAGGGCTGGGGGAAATGGGCAGCTCCAGTA
TTGGCCATTTTCTAGTTCAGACCTGTACAACTGGAAGAACAATAACCCCTCTTTCAGTGAGGACCCCG
GCAAACTGACCGCCCTGATCGAATCCGTGCTGATTACCCATCAGCCCACATGGGACGATTGTCAGCAG
CTCCTGGGCACCCTGCTGACCGGAGAGGAAAAGCAGCGCGTGCTGCTGGAGGCTCGCAAAGCAGTCCG
AGGGGACGATGGACGGCCCACACAGCTCCCTAATGAGGTGGACGCCGCTTTTCCACTGGAAAGACCCG
ACTGGGATTATACTACCCAGGCAGGGAGAAACCACCTGGTCCATTACAGGCAGCTCCTGCTGGCAGGC
CTGCAGAATGCCGGGAGATCCCCCACCAACCTGGCCAAGGTGAAAGGCATCACACAGGGGCCTAATGA
GTCACCAAGCGCCTTTCTGGAGAGGCTGAAGGAAGCTTACCGACGGTATACCCCATACGACCCTGAGG
ACCCCGGACAGGAAACAAACGTCTCCATGTCTTTCATCTGGCAGTCTGCCCCAGACATTGGGCGGAAG
CTGGAGAGACTGGAAGACCTGAAGAACAAGACCCTGGGCGACCTGGTGCGGGAGGCTGAAAAGATCTT
CAACAAACGGGAGACCCCCGAGGAAAGAGAGGAAAGGATTAGAAGGGAAACTGAGGAAAAGGAGGAAC
GCCGACGGACCGAGGACGAACAGAAGGAGAAAGAACGAGATCGGCGGCGGCACCGGGAGATGTCAAAG
CTGCTGGCCACCGTGGTCAGCGGACAGAAACAGGACAGACAGGGAGGAGAGCGACGGAGAAGCCAGCT
CGACAGGGATCAGTGCGCATACTGTAAGGAAAAAGGCCATTGGGCCAAGGATTGCCCCAAAAAGCCAA
GAGGACCAAGAGGACCAAGACCACAGACATCACTGCTGACCCTGGACGACTAA
SEQ ID NO: 4 is a MMLV-Gag mRNA Sequence (codon optimized)
AUGGGACAGACCGUCACAACACCCCUGAGCCUGACCCUGGGACAUUGGAAAGACGUGGAGAGGAUCGC
ACAUAACCAGAGCGUGGACGUGAAGAAACGGAGAUGGGUCACAUUCUGCAGUGCUGAGUGGCCAACUU
UUAAUGUGGGAUGGCCCCGAGACGGCACUUUCAACAGGGAUCUGAUCACCCAGGUGAAGAUCAAGGUC
UUUAGCCCAGGACCUCACGGACAUCCAGACCAGGUGCCUUAUAUCGUCACCUGGGAGGCACUGGCCUU
CGAUCCCCCUCCAUGGGUGAAGCCAUUUGUCCACCCAAAACCACCUCCACCACUGCCUCCAAGUGCCC
CUUCACUGCCACUGGAACCACCCCGGAGCACACCACCCCGCAGCUCCCUGUAUCCUGCUCUGACUCCA
UCUCUGGGCGCAAAGCCAAAACCACAGGUGCUGAGCGACUCCGGAGGACCACUGAUUGACCUGCUGAC
AGAGGACCCCCCACCAUACCGAGAUCCUCGGCCUCCACCAAGCGACCGCGAUGGAAAUGGAGGAGAGG
CUACUCCUGCCGGCGAAGCCCCUGACCCAUCUCCAAUGGCUAGUAGGCUGCGCGGCAGGCGCGAGCCU
CCAGUGGCAGAUAGCACCACAUCCCAGGCCUUCCCUCUGAGGGCUGGGGGAAAUGGGCAGCUCCAGUA
UUGGCCAUUUUCUAGUUCAGACCUGUACAACUGGAAGAACAAUAACCCCUCUUUCAGUGAGGACCCCG
GCAAACUGACCGCCCUGAUCGAAUCCGUGCUGAUUACCCAUCAGCCCACAUGGGACGAUUGUCAGCAG
CUCCUGGGCACCCUGCUGACCGGAGAGGAAAAGCAGCGCGUGCUGCUGGAGGCUCGCAAAGCAGUCCG
AGGGGACGAUGGACGGCCCACACAGCUCCCUAAUGAGGUGGACGCCGCUUUUCCACUGGAAAGACCCG
ACUGGGAUUAUACUACCCAGGCAGGGAGAAACCACCUGGUCCAUUACAGGCAGCUCCUGCUGGCAGGC
CUGCAGAAUGCCGGGAGAUCCCCCACCAACCUGGCCAAGGUGAAAGGCAUCACACAGGGGCCUAAUGA
GUCACCAAGCGCCUUUCUGGAGAGGCUGAAGGAAGCUUACCGACGGUAUACCCCAUACGACCCUGAGG
ACCCCGGACAGGAAACAAACGUCUCCAUGUCUUUCAUCUGGCAGUCUGCCCCAGACAUUGGGCGGAAG
CUGGAGAGACUGGAAGACCUGAAGAACAAGACCCUGGGCGACCUGGUGCGGGAGGCUGAAAAGAUCUU
CAACAAACGGGAGACCCCCGAGGAAAGAGAGGAAAGGAUUAGAAGGGAAACUGAGGAAAAGGAGGAAC
GCCGACGGACCGAGGACGAACAGAAGGAGAAAGAACGAGAUCGGCGGCGGCACCGGGAGAUGUCAAAG
CUGCUGGCCACCGUGGUCAGCGGACAGAAACAGGACAGACAGGGAGGAGAGCGACGGAGAAGCCAGCU
CGACAGGGAUCAGUGCGCAUACUGUAAGGAAAAAGGCCAUUGGGCCAAGGAUUGCCCCAAAAAGCCAA
GAGGACCAAGAGGACCAAGACCACAGACAUCACUGCUGACCCUGGACGACUAA

Typically in nature, a Gag protein includes a large C-terminal extension which may contain retroviral protease, reverse transcriptase, and integrase enzymatic activity. Assembly of VLPs, however, generally does not require the presence of such components. In some cases, a retroviral Gag protein alone (e.g., lacking a C-terminal extension, lacking one or more of genomic RNA, reverse transcriptase, viral protease, or envelope protein) can self-assemble to form VLPs both in vitro and in vivo (Sharma S et al., 1997 Proc. Natl. Acad. Sci. USA 94:10803-8). Retroviral Gag polyprotein alone can oligomerize and assemble into VLPs.

In some embodiments, a Gag polypeptide for use as described herein lacks a C-terminal extension and/or contains a modified C-terminal extension. A Gag polypeptide may optionally include one or more additional polypeptides (e.g., a heterologous antigen). In some embodiments, a Gag polypeptide is co-expressed with a heterologous antigen (e.g., under separate promoters and/or as separate proteins). In some embodiments, a Gag polypeptide is expressed as a fusion protein with a heterologous antigen. The Gag polypeptide can be linked to a heterologous antigen to create a fusion protein without altering Gag function. For example, a coding sequence for a heterologous antigen may be spliced into the Gag polypeptide coding sequence, e.g., at the 3′ end of the Gag polypeptide coding sequence. In some embodiments, a coding sequence for a heterologous antigen may be spliced in frame into the Gag polypeptide coding sequence. In some embodiments, a Gag polypeptide-coding sequence and heterologous antigen may be expressed by a single promoter. In some embodiments, a heterologous antigen is inserted at (e.g., fused to) the C-terminus of a Gag polypeptide. Without wishing to be bound by any theory, it is thought that fusion of a self-assembling Gag polypeptide to a heterologous antigen creates a fusion protein that acts as unmodified Gag and as a result will allow the antigen to be incorporated into the structural components of a resulting VLP. In some embodiments, VLP structural components serve as effective immunogens (e.g., for induction of cellular immune response). For example, provided VLPs may comprise a retroviral Gag polypeptide (e.g., MMLV Gag) and a structural component of EBV (e.g., EBNA1). In some such embodiments, EBNA1 is incorporated into the VLP and serves as an antigen for eliciting an immune response against EBV.

In some instances, a Gag polypeptide may be fused with a heterologous structural component that has been modified. In some cases, the modified structural protein may be truncated while in other instances it may be mutated. In some instances, a Gag polypeptide may be fused with a heterologous envelope glycoprotein in which the transmembrane and cytoplasmic tails have been truncated and/or deleted.

In some embodiments, a suitable Gag polypeptide fusion protein is substantially identical and/or substantially homologous to a retroviral Gag polypeptide. For example, SEQ ID NO: 5. A Gag polypeptide fusion protein may be a modified retroviral Gag polypeptide containing one or more amino acid substitutions, deletions, and/or insertions as compared to a wild-type or naturally-occurring Gag polypeptide, while retaining substantial self-assembly activity. Thus, in some embodiments, a Gag polypeptide suitable for use herein is substantially identical to and/or substantially homologous to such a sequence as SEQ ID NO: 5. In some embodiments, a Gag polypeptide suitable for use herein has an amino acid sequence of at least 50%, 55%, 60%, 65%, 70%, 75%, 80%, 85%, 90%, 91%, 92%, 93%, 94%, 95%, 96%, 97%, 98%, 99% identity to SEQ ID NO:5.

SEQ ID NO: 5 is a MMLV-Gag/EBV EBNA1 Amino Acid Sequence
MGQTVTTPLSLTLGHWKDVERIAHNQSVDVKKRRWVTFCSAEWPTENVGWPRDGTFNRDLITQVKIKV
FSPGPHGHPDQVPYIVTWEALAFDPPPWVKPFVHPKPPPPLPPSAPSLPLEPPRSTPPRSSLYPALTP
SLGAKPKPQVLSDSGGPLIDLLTEDPPPYRDPRPPPSDRDGNGGEATPAGEAPDPSPMASRLRGRREP
PVADSTTSQAFPLRAGGNGQLQYWPESSSDLYNWKNNNPSFSEDPGKLTALIESVLITHQPTWDDCQQ
LLGTLLTGEEKQRVLLEARKAVRGDDGRPTQLPNEVDAAFPLERPDWDYTTQAGRNHLVHYRQLLLAG
LQNAGRSPTNLAKVKGITQGPNESPSAFLERLKEAYRRYTPYDPEDPGQETNVSMSFIWQSAPDIGRK
LERLEDLKNKTLGDLVREAEKIENKRETPEEREERIRRETEEKEERRRTEDEQKEKERDRRRHREMSK
LLATVVSGQKQDRQGGERRRSQLDRDQCAYCKEKGHWAKDCPKKPRGPRGPRPQTSLLTLDDMSDEGP
GTGPGNGLGQKEDTSGPEGSGGSGPQRRGGDNHGRGRGRGRGRGGGRPGAPGGSGSGPRHRDGVRRPQ
KRPSCIGCKGAHRRGRGRERARGGSRERARGRGRGRGEKRPRSPSSQSSSSGSPPRRPPPGRRPFFHP
VGEADYFEYHQEGGPDGEPDVPPGAIEQGPADDPGEGPSTGPRGQGDGGRRKKGGWFGKHRGQGGSNP
KFENIAEGLRALLARSHVERTTDEGTWVAGVFVYGGSKTSLYNLRRGIALAIPQCRLTPLSRLPFGMA
PGPGPQPGPLRESIVCYFMVELQTHIFAEVLKDAIKDLVMTKPAPTCNIKVTVCSFDDGVDLPPWEPP
MVEGAAAEGDDGDDGDEGGDGDEGEEGQE
SEQ ID NO: 6 is a MMLV-Gag/EBV EBNA1 Nucleotide Sequence
ATGGGCCAGACTGTTACCACTCCCTTAAGTTTGACCTTAGGTCACTGGAAAGATGTCGAGCGGATCGC
TCACAACCAGTCGGTAGATGTCAAGAAGAGACGTTGGGTTACCTTCTGCTCTGCAGAATGGCCAACCT
TTAACGTCGGATGGCCGCGAGACGGCACCTTTAACCGAGACCTCATCACCCAGGTTAAGATCAAGGTC
TTTTCACCTGGCCCGCATGGACACCCAGACCAGGTCCCCTACATCGTGACCTGGGAAGCCTTGGCTTT
TGACCCCCCTCCCTGGGTCAAGCCCTTTGTACACCCTAAGCCTCCGCCTCCTCTTCCTCCATCCGCCC
CGTCTCTCCCCCTTGAACCTCCTCGTTCGACCCCGCCTCGATCCTCCCTTTATCCAGCCCTCACTCCT
TCTCTAGGCGCCAAACCTAAACCTCAAGTTCTTTCTGACAGTGGGGGGCCGCTCATCGACCTACTTAC
AGAAGACCCCCCGCCTTATAGGGACCCAAGACCACCCCCTTCCGACAGGGACGGAAATGGTGGAGAAG
CGACCCCTGCGGGAGAGGCACCGGACCCCTCCCCAATGGCATCTCGCCTACGTGGGAGACGGGAGCCC
CCTGTGGCCGACTCCACTACCTCGCAGGCATTCCCCCTCCGCGCAGGAGGAAACGGACAGCTTCAATA
CTGGCCGTTCTCCTCTTCTGACCTTTACAACTGGAAAAATAATAACCCTTCTTTTTCTGAAGATCCAG
GTAAACTGACAGCTCTGATCGAGTCTGTTCTCATCACCCATCAGCCCACCTGGGACGACTGTCAGCAG
CTGTTGGGGACTCTGCTGACCGGAGAAGAAAAACAACGGGTGCTCTTAGAGGCTAGAAAGGCGGTGCG
GGGCGATGATGGGCGCCCCACTCAACTGCCCAATGAAGTCGATGCCGCTTTTCCCCTCGAGCGCCCAG
ACTGGGATTACACCACCCAGGCAGGTAGGAACCACCTAGTCCACTATCGCCAGTTGCTCCTAGCGGGT
CTCCAAAACGCGGGCAGAAGCCCCACCAATTTGGCCAAGGTAAAAGGAATAACACAAGGGCCCAATGA
GTCTCCCTCGGCCTTCCTAGAGAGACTTAAGGAAGCCTATCGCAGGTACACTCCTTATGACCCTGAGG
ACCCAGGGCAAGAAACTAATGTGTCTATGTCTTTCATTTGGCAGTCTGCCCCAGACATTGGGAGAAAG
TTAGAGAGGTTAGAAGATTTAAAAAACAAGACGCTTGGAGATTTGGTTAGAGAGGCAGAAAAGATCTT
TAATAAACGAGAAACCCCGGAAGAAAGAGAGGAACGTATCAGGAGAGAAACAGAGGAAAAAGAAGAAC
GCCGTAGGACAGAGGATGAGCAGAAAGAGAAAGAAAGAGATCGTAGGAGACATAGAGAGATGAGCAAG
CTATTGGCCACTGTCGTTAGTGGACAGAAACAGGATAGACAGGGAGGAGAACGAAGGAGGTCCCAACT
CGATCGCGACCAGTGTGCCTACTGCAAAGAAAAGGGGCACTGGGCTAAAGATTGTCCCAAGAAACCAC
GAGGACCTCGGGGACCAAGACCCCAGACCTCCCTCCTGACCCTAGATGACATGTCTGACGAGGGGCCA
GGTACAGGACCTGGAAATGGCCTAGGACAGAAGGAGGACACATCTGGACCAGAAGGCTCCGGCGGCAG
TGGACCTCAAAGAAGAGGGGGTGATAACCATGGACGAGGACGGGGAAGAGGACGAGGACGAGGAGGCG
GAAGACCAGGAGCCCCGGGCGGCTCAGGATCAGGGCCAAGACATAGAGATGGTGTCCGGAGACCCCAA
AAACGTCCAAGTTGCATTGGCTGCAAAGGGGCCCACCGCCGGGGTAGAGGACGTGAAAGAGCCAGGGG
GGGAAGTCGTGAAAGAGCCAGGGGGAGAGGTCGTGGACGTGGAGAAAAGAGGCCCAGGAGTCCCAGTA
GTCAGTCATCATCATCCGGGTCTCCACCGCGCAGGCCCCCTCCAGGTAGAAGGCCATTTTTCCACCCT
GTAGGGGAAGCCGATTATTTTGAATACCACCAAGAAGGTGGCCCAGATGGTGAGCCTGACGTGCCCCC
GGGAGCGATAGAGCAGGGCCCCGCAGATGACCCAGGAGAAGGCCCAAGCACTGGACCCCGGGGTCAGG
GTGATGGAGGCAGGCGCAAAAAAGGAGGGTGGTTTGGAAAGCATCGTGGTCAAGGAGGTTCCAACCCG
AAATTTGAGAACATTGCAGAAGGTTTAAGAGCTCTCCTGGCTAGGAGTCACGTAGAAAGGACTACCGA
CGAAGGAACTTGGGTCGCCGGTGTGTTCGTATATGGAGGTAGTAAGACCTCCCTTTACAACCTAAGGC
GAGGAATTGCCCTTGCTATTCCACAATGTCGTCTTACACCATTGAGTCGTCTCCCCTTTGGAATGGCC
CCTGGACCCGGCCCACAACCTGGCCCGCTAAGGGAGTCCATTGTCTGTTATTTCATGGTCTTTTTACA
AACTCATATATTTGCTGAGGTTTTGAAGGATGCGATTAAGGACCTTGTTATGACAAAGCCCGCTCCTA
CCTGCAATATCAAAGTGACTGTGTGCAGCTTTGACGATGGAGTAGATTTGCCTCCCTGGTTTCCACCT
ATGGTGGAAGGGGCTGCCGCGGAGGGTGATGACGGAGATGACGGAGATGAAGGAGGTGATGGAGATGA
GGGTGAGGAAGGGCAGGAGTGA
SEQ ID NO: 7 is a MMLV-Gag/EBV EBNA1 Codon Optimized Nucleotide Sequence
ATGGGGCAGACAGTGACAACTCCTCTGAGCCTGACCCTGGGGCATTGGAAAGACGTGGAACGAATCGC
ACATAACCAGTCCGTGGATGTGAAGAAGCGCAGGTGGGTGACCTTCTGTTCTGCCGAGTGGCCAACCT
TCAACGTGGGCTGGCCCCGGGATGGCACCTTTAATCGGGATCTGATCACACAGGTGAAGATCAAGGTG
TTTTCTCCAGGCCCACACGGCCACCCTGATCAGGTGCCCTATATCGTGACATGGGAGGCCCTGGCCTT
CGACCCCCCCCCATGGGTGAAGCCCTTCGTGCACCCAAAGCCCCCCCCCCCCCTGCCCCCATCCGCCC
CCTCCCTGCCCCTGGAGCCACCCAGGTCCACCCCCCCCAGAAGCAGCCTGTACCCTGCCCTGACCCCA
TCCCTGGGCGCGAAGCCCAAGCCACAGGTGCTGAGCGATTCTGGCGGCCCACTGATCGACCTGCTGAC
AGAGGACCCACCCCCATATCGGGACCCTCGCCCTCCTCCATCTGATAGAGACGGCAATGGCGGCGAGG
CCACACCCGCCGGCGAGGCCCCCGATCCTAGCCCCATGGCCTCCCGCCTGAGGGGCAGAAGGGAGCCA
CCCGTGGCCGACAGCACAACCTCTCAGGCCTTTCCTCTGCGGGCCGGCGGCAACGGCCAGCTACAGTA
TTGGCCCTTCAGCAGCTCTGATCTGTACAACTGGAAGAACAATAACCCTTCCTTTTCTGAGGACCCCG
GCAAGCTGACCGCCCTGATCGAGTCTGTGCTGATCACCCACCAGCCCACCTGGGATGACTGTCAGCAG
CTACTGGGCACACTGCTGACAGGCGAGGAGAAGCAGCGGGTGCTGCTGGAGGCCAGAAAGGCCGTGCG
GGGCGATGACGGCAGGCCAACACAGCTACCCAACGAGGTGGATGCCGCCTTTCCTCTGGAGAGGCCAG
ACTGGGACTATACAACCCAGGCCGGGCGCAACCACCTGGTGCACTACCGGCAGCTACTGCTGGCCGGC
CTGCAGAACGCCGGCAGGTCCCCCACAAATCTGGCCAAGGTGAAGGGCATCACACAGGGCCCTAACGA
GTCTCCCTCTGCCTTCCTGGAGAGACTGAAGGAGGCCTACCGGAGATACACACCCTATGATCCAGAGG
ACCCAGGCCAGGAGACAAATGTATCCATGTCCTTCATCTGGCAGTCCGCCCCAGACATCGGCAGAAAG
CTGGAGCGGCTGGAGGACCTGAAGAACAAGACCCTGGGCGACCTGGTGCGGGAGGCCGAGAAGATCTT
CAATAAGCGCGAGACCCCTGAGGAGCGGGAGGAGAGAATCAGACGCGAGACCGAGGAGAAGGAGGAGA
GGAGGAGGACCGAGGACGAGCAGAAGGAGAAGGAGAGAGATAGAAGGCGGCACCGGGAGATGTCCAAG
CTGCTGGCCACCGTGGTGTCCGGCCAGAAGCAGGACCGGCAGGGCGGCGAGCGCCGGAGATCTCAGCT
AGACCGCGATCAGTGCGCCTACTGTAAGGAGAAGGGCCACTGGGCCAAGGACTGCCCCAAGAAGCCAA
GGGGCCCCCGGGGCCCAAGGCCTCAGACCTCCCTGCTGACCCTGGACGACATGTCCGATGAGGGCCCC
GGCACAGGCCCAGGCAACGGCCTGGGCCAGAAGGAGGACACCTCCGGCCCTGAGGGCTCCGGCGGCAG
CGGCCCTCAGAGGAGGGGCGGCGACAATCACGGCAGAGGCCGGGGCCGGGGCCGGGGCAGGGGCGGCG
GCAGACCAGGCGCGCCCGGCGGCTCCGGCAGCGGCCCCAGGCACAGAGATGGCGTGCGCAGACCCCAG
AAGCGGCCTTCTTGTATCGGCTGCAAGGGCGCGCACAGGAGAGGCCGGGGCCGGGAGAGGGCCCGGGG
CGGCAGCAGGGAGCGCGCCAGAGGCCGGGGCAGAGGCAGAGGCGAGAAGAGACCACGGTCTCCCTCTT
CCCAGTCCTCCTCTAGCGGCTCCCCCCCACGCCGGCCTCCCCCCGGGCGGAGACCATTTTTCCACCCA
GTGGGCGAGGCCGATTACTTTGAGTATCACCAGGAGGGCGGCCCTGATGGCGAGCCCGATGTGCCACC
CGGCGCGATCGAGCAGGGCCCCGCCGACGATCCAGGCGAGGGCCCCAGCACCGGCCCCAGGGGCCAGG
GCGACGGCGGCAGACGCAAGAAGGGCGGCTGGTTTGGCAAGCACCGGGGCCAGGGCGGCTCCAATCCA
AAGTTTGAGAATATCGCCGAGGGCCTGCGGGCCCTGCTGGCCAGATCCCACGTAGAGAGGACCACCGA
CGAGGGCACCTGGGTGGCCGGCGTGTTTGTATATGGCGGCTCTAAGACAAGCCTGTACAACCTGCGGA
GAGGCATCGCCCTGGCCATCCCACAGTGTAGACTGACACCACTGAGCAGACTGCCATTCGGCATGGCC
CCTGGCCCAGGCCCTCAGCCCGGCCCTCTGAGAGAGTCCATCGTGTGCTACTTCATGGTGTTCCTGCA
GACCCACATCTTTGCCGAGGTGCTGAAGGATGCCATCAAGGACCTGGTAATGACAAAGCCTGCCCCCA
CATGCAATATCAAGGTGACAGTGTGCTCCTTTGACGACGGCGTGGACCTGCCCCCATGGTTTCCCCCA
ATGGTGGAGGGCGCAGCCGCCGAGGGCGACGATGGCGACGATGGGGACGAGGGAGGCGACGGCGACGA
GGGAGAAGAAGGACAGGAGTAA
SEQ ID NO: 8 is a MMLV-Gag/EBV EBNA1 mRNA Sequence
AUGGGGCAGACAGUGACAACUCCUCUGAGCCUGACCCUGGGGCAUUGGAAAGACGUGGAACGAAUCGC
ACAUAACCAGUCCGUGGAUGUGAAGAAGCGCAGGUGGGUGACCUUCUGUUCUGCCGAGUGGCCAACCU
UCAACGUGGGCUGGCCCCGGGAUGGCACCUUUAAUCGGGAUCUGAUCACACAGGUGAAGAUCAAGGUG
UUUUCUCCAGGCCCACACGGCCACCCUGAUCAGGUGCCCUAUAUCGUGACAUGGGAGGCCCUGGCCUU
CGACCCCCCCCCAUGGGUGAAGCCCUUCGUGCACCCAAAGCCCCCCCCCCCCCUGCCCCCAUCCGCCC
CCUCCCUGCCCCUGGAGCCACCCAGGUCCACCCCCCCCAGAAGCAGCCUGUACCCUGCCCUGACCCCA
UCCCUGGGCGCGAAGCCCAAGCCACAGGUGCUGAGCGAUUCUGGCGGCCCACUGAUCGACCUGCUGAC
AGAGGACCCACCCCCAUAUCGGGACCCUCGCCCUCCUCCAUCUGAUAGAGACGGCAAUGGCGGCGAGG
CCACACCCGCCGGCGAGGCCCCCGAUCCUAGCCCCAUGGCCUCCCGCCUGAGGGGCAGAAGGGAGCCA
CCCGUGGCCGACAGCACAACCUCUCAGGCCUUUCCUCUGCGGGCCGGCGGCAACGGCCAGCUACAGUA
UUGGCCCUUCAGCAGCUCUGAUCUGUACAACUGGAAGAACAAUAACCCUUCCUUUUCUGAGGACCCCG
GCAAGCUGACCGCCCUGAUCGAGUCUGUGCUGAUCACCCACCAGCCCACCUGGGAUGACUGUCAGCAG
CUACUGGGCACACUGCUGACAGGCGAGGAGAAGCAGCGGGUGCUGCUGGAGGCCAGAAAGGCCGUGCG
GGGCGAUGACGGCAGGCCAACACAGCUACCCAACGAGGUGGAUGCCGCCUUUCCUCUGGAGAGGCCAG
ACUGGGACUAUACAACCCAGGCCGGGCGCAACCACCUGGUGCACUACCGGCAGCUACUGCUGGCCGGC
CUGCAGAACGCCGGCAGGUCCCCCACAAAUCUGGCCAAGGUGAAGGGCAUCACACAGGGCCCUAACGA
GUCUCCCUCUGCCUUCCUGGAGAGACUGAAGGAGGCCUACCGGAGAUACACACCCUAUGAUCCAGAGG
ACCCAGGCCAGGAGACAAAUGUAUCCAUGUCCUUCAUCUGGCAGUCCGCCCCAGACAUCGGCAGAAAG
CUGGAGCGGCUGGAGGACCUGAAGAACAAGACCCUGGGCGACCUGGUGCGGGAGGCCGAGAAGAUCUU
CAAUAAGCGCGAGACCCCUGAGGAGCGGGAGGAGAGAAUCAGACGCGAGACCGAGGAGAAGGAGGAGA
GGAGGAGGACCGAGGACGAGCAGAAGGAGAAGGAGAGAGAUAGAAGGCGGCACCGGGAGAUGUCCAAG
CUGCUGGCCACCGUGGUGUCCGGCCAGAAGCAGGACCGGCAGGGCGGCGAGCGCCGGAGAUCUCAGCU
AGACCGCGAUCAGUGCGCCUACUGUAAGGAGAAGGGCCACUGGGCCAAGGACUGCCCCAAGAAGCCAA
GGGGCCCCCGGGGCCCAAGGCCUCAGACCUCCCUGCUGACCCUGGACGACAUGUCCGAUGAGGGCCCC
GGCACAGGCCCAGGCAACGGCCUGGGCCAGAAGGAGGACACCUCCGGCCCUGAGGGCUCCGGCGGCAG
CGGCCCUCAGAGGAGGGGCGGCGACAAUCACGGCAGAGGCCGGGGCCGGGGCCGGGGCAGGGGCGGCG
GCAGACCAGGCGCGCCCGGCGGCUCCGGCAGCGGCCCCAGGCACAGAGAUGGCGUGCGCAGACCCCAG
AAGCGGCCUUCUUGUAUCGGCUGCAAGGGCGCGCACAGGAGAGGCCGGGGCCGGGAGAGGGCCCGGGG
CGGCAGCAGGGAGCGCGCCAGAGGCCGGGGCAGAGGCAGAGGCGAGAAGAGACCACGGUCUCCCUCUU
CCCAGUCCUCCUCUAGCGGCUCCCCCCCACGCCGGCCUCCCCCCGGGCGGAGACCAUUUUUCCACCCA
GUGGGCGAGGCCGAUUACUUUGAGUAUCACCAGGAGGGCGGCCCUGAUGGCGAGCCCGAUGUGCCACC
CGGCGCGAUCGAGCAGGGCCCCGCCGACGAUCCAGGCGAGGGCCCCAGCACCGGCCCCAGGGGCCAGG
GCGACGGCGGCAGACGCAAGAAGGGCGGCUGGUUUGGCAAGCACCGGGGCCAGGGCGGCUCCAAUCCA
AAGUUUGAGAAUAUCGCCGAGGGCCUGCGGGCCCUGCUGGCCAGAUCCCACGUAGAGAGGACCACCGA
CGAGGGCACCUGGGUGGCCGGCGUGUUUGUAUAUGGCGGCUCUAAGACAAGCCUGUACAACCUGCGGA
GAGGCAUCGCCCUGGCCAUCCCACAGUGUAGACUGACACCACUGAGCAGACUGCCAUUCGGCAUGGCC
CCUGGCCCAGGCCCUCAGCCCGGCCCUCUGAGAGAGUCCAUCGUGUGCUACUUCAUGGUGUUCCUGCA
GACCCACAUCUUUGCCGAGGUGCUGAAGGAUGCCAUCAAGGACCUGGUAAUGACAAAGCCUGCCCCCA
CAUGCAAUAUCAAGGUGACAGUGUGCUCCUUUGACGACGGCGUGGACCUGCCCCCAUGGUUUCCCCCA
AUGGUGGAGGGCGCAGCCGCCGAGGGCGACGAUGGCGACGAUGGGGACGAGGGAGGCGACGGCGACGA
GGGAGAAGAAGGACAGGAGUAA
SEQ ID NO: 9 is a MMLV-Gag/EBV gp350 Amino Acid Sequence (Construct:
Gag fused with truncated gp350[22-434])
MGQTVTTPLSLTLGHWKDVERIAHNQSVDVKKRRWVTFCSAEWPTFNVGWPRDGTFNRDLITQVKIKV
FSPGPHGHPDQVPYIVTWEALAFDPPPWVKPFVHPKPPPPLPPSAPSLPLEPPRSTPPRSSLYPALTP
SLGAKPKPQVLSDSGGPLIDLLTEDPPPYRDPRPPPSDRDGNGGEATPAGEAPDPSPMASRLRGRREP
PVADSTTSQAFPLRAGGNGQLQYWPFSSSDLYNWKNNNPSFSEDPGKLTALIESVLITHQPTWDDCQQ
LLGTLLTGEEKQRVLLEARKAVRGDDGRPTQLPNEVDAAFPLERPDWDYTTQAGRNHLVHYRQLLLAG
LQNAGRSPTNLAKVKGITQGPNESPSAFLERLKEAYRRYTPYDPEDPGQETNVSMSFIWQSAPDIGRK
LERLEDLKNKTLGDLVREAEKIENKRETPEEREERIRRETEEKEERRRTEDEQKEKERDRRRHREMSK
LLATVVSGQKQDRQGGERRRSQLDRDQCAYCKEKGHWAKDCPKKPRGPRGPRPQTSLLTLDDDPGEEN
VEIPEFPFYPTCNVCTADVNVTINFDVGGKKHQLDLDFGQLTPHTKAVYQPRGAFGGSENATNLELLE
LLGAGELALTMRSKKLPINVTTGEEQQVSLESVDVYFQDVFGTMWCHHAEMQNPVYLIPETVPYIKWD
NCNSTNITAVVRAQGLDVTLPLSLPTSAQDSNFSVKTQMLGNEIDIECIMEDGEISQVLPGDNKENIT
CSGYESHVPSGGILTSTSPVATPIPGTGYAYSLRLTPRPVSRELGNNSILYVFYSGNGPKASGGDYCI
QSNIVESDEIPASQDMPTNTTDITYVGDNATYSVPMVTSEDANSPNVTVTAFWAWPNNTETDEKCKWT
LTSGTPSGCENISGAFASNRTFDITVSGLGTAPKTLIITRTATNATTTTHKVIFSKAPESTTTSPTLN
TTGFADPNTTTGLPSSTHVPTNLTAPASTGPTVSTADVTSPTPAGTTSGASPVTPSPSPRDNGTESKA
PDMTSSTSPVTTPTPNATSPTPAVTTPTPNATSPTPAVTTPTPNATSPTLGKTSPTSAVTTPTPNATS
PTLGKTSPTSAVTTPTPNATSPTLGKTSPTSAVTTPTPNATGPTVGETSPQANATNHTLGGTSPTPVV
TSQPKNATSAVTTGQHNITSSSTSSMSLRPSSIPETLSPSTSDNSTSHMPLLTSAHPTGGENITQVTP
ASISTHHVSTSSPAPRPGTTSQASGPGNSSTSTKPGEVNVTKGTPPQNATSPQAPSGQKTAVPTVTST
GGKANSTTGGKHTTGHGARTSTEPTTDYGGDSTTPRPRYNATTYLPPSTSSKLRPRWTFTSPPVTTAQ
ATVPVPPTSQPRES
SEQ ID NO: 10 is a MMLV-Gag/EBV gp350 Nucleotide Sequence
ATGGGACAGACCGTCACAACACCCCTGAGCCTGACCCTGGGACATTGGAAAGACGTGGAGAGGATCGC
ACATAACCAGAGCGTGGACGTGAAGAAACGGAGATGGGTCACATTCTGCAGTGCTGAGTGGCCAACTT
TTAATGTGGGATGGCCCCGAGACGGCACTTTCAACAGGGATCTGATCACCCAGGTGAAGATCAAGGTC
TTTAGCCCAGGACCTCACGGACATCCAGACCAGGTGCCTTATATCGTCACCTGGGAGGCACTGGCCTT
CGATCCCCCTCCATGGGTGAAGCCATTTGTCCACCCAAAACCACCTCCACCACTGCCTCCAAGTGCCC
CTTCACTGCCACTGGAACCACCCCGGAGCACACCACCCCGCAGCTCCCTGTATCCTGCTCTGACTCCA
TCTCTGGGCGCAAAGCCAAAACCACAGGTGCTGAGCGACTCCGGAGGACCACTGATTGACCTGCTGAC
AGAGGACCCCCCACCATACCGAGATCCTCGGCCTCCACCAAGCGACCGCGATGGAAATGGAGGAGAGG
CTACTCCTGCCGGCGAAGCCCCTGACCCATCTCCAATGGCTAGTAGGCTGCGCGGCAGGCGCGAGCCT
CCAGTGGCAGATAGCACCACATCCCAGGCCTTCCCTCTGAGGGCTGGGGGAAATGGGCAGCTCCAGTA
TTGGCCATTTTCTAGTTCAGACCTGTACAACTGGAAGAACAATAACCCCTCTTTCAGTGAGGACCCCG
GCAAACTGACCGCCCTGATCGAATCCGTGCTGATTACCCATCAGCCCACATGGGACGATTGTCAGCAG
CTCCTGGGCACCCTGCTGACCGGAGAGGAAAAGCAGCGCGTGCTGCTGGAGGCTCGCAAAGCAGTCCG
AGGGGACGATGGACGGCCCACACAGCTCCCTAATGAGGTGGACGCCGCTTTTCCACTGGAAAGACCCG
ACTGGGATTATACTACCCAGGCAGGGAGAAACCACCTGGTCCATTACAGGCAGCTCCTGCTGGCAGGC
CTGCAGAATGCCGGGAGATCCCCCACCAACCTGGCCAAGGTGAAAGGCATCACACAGGGGCCTAATGA
GTCACCAAGCGCCTTTCTGGAGAGGCTGAAGGAAGCTTACCGACGGTATACCCCATACGACCCTGAGG
ACCCCGGACAGGAAACAAACGTCTCCATGTCTTTCATCTGGCAGTCTGCCCCAGACATTGGGCGGAAG
CTGGAGAGACTGGAAGACCTGAAGAACAAGACCCTGGGCGACCTGGTGCGGGAGGCTGAAAAGATCTT
CAACAAACGGGAGACCCCCGAGGAAAGAGAGGAAAGGATTAGAAGGGAAACTGAGGAAAAGGAGGAAC
GCCGACGGACCGAGGACGAACAGAAGGAGAAAGAACGAGATCGGCGGCGGCACCGGGAGATGTCAAAG
CTGCTGGCCACCGTGGTCAGCGGACAGAAACAGGACAGACAGGGAGGAGAGCGACGGAGAAGCCAGCT
CGACAGGGATCAGTGCGCATACTGTAAGGAAAAAGGCCATTGGGCCAAGGATTGCCCCAAAAAGCCAA
GAGGACCAAGAGGACCAAGACCACAGACATCACTGCTGACCCTGGACGACGATCCTGGGTTTTTCAAT
GTGGAGATCCCTGAGTTTCCATTCTACCCAACATGCAACGTGTGCACCGCCGACGTGAACGTGACCAT
CAATTTTGATGTGGGCGGCAAGAAGCACCAGCTAGACCTGGATTTTGGCCAGCTAACACCTCACACCA
AGGCCGTGTATCAGCCACGCGGCGCGTTTGGCGGCAGCGAGAACGCCACAAACCTGTTTCTGCTGGAG
CTGCTGGGCGCGGGCGAGCTGGCCCTGACCATGAGAAGCAAGAAGCTGCCAATCAACGTGACCACCGG
CGAGGAGCAGCAGGTGTCCCTGGAGTCCGTGGACGTGTACTTCCAGGACGTGTTCGGCACAATGTGGT
GCCACCACGCCGAGATGCAGAACCCAGTGTACCTGATCCCAGAGACCGTGCCATACATCAAGTGGGAC
AACTGCAACTCCACCAATATCACAGCCGTGGTGCGGGCCCAGGGCCTGGATGTGACACTGCCTCTGAG
CCTGCCTACATCTGCCCAGGACAGCAACTTTTCCGTGAAGACCCAGATGCTGGGCAACGAGATCGACA
TCGAGTGCATCATGGAGGATGGCGAGATCTCCCAGGTGCTGCCTGGCGACAATAAGTTCAACATCACA
TGTAGCGGCTACGAGAGCCACGTACCTTCCGGCGGCATCCTGACCTCTACAAGCCCAGTGGCCACCCC
AATCCCCGGCACCGGCTATGCCTACTCTCTGAGGCTGACACCTCGGCCCGTGAGCAGATTCCTGGGCA
ACAACTCCATCCTGTACGTGTTCTACTCCGGCAATGGCCCTAAGGCCAGCGGCGGCGACTACTGTATC
CAGAGCAACATCGTGTTTTCCGACGAGATCCCTGCCTCTCAGGATATGCCCACCAATACCACCGACAT
CACATACGTGGGCGACAATGCCACATATAGCGTGCCAATGGTGACCAGCGAGGACGCCAATAGCCCTA
ACGTGACAGTGACCGCCTTTTGGGCCTGGCCCAACAATACAGAGACCGATTTCAAGTGTAAGTGGACC
CTGACCAGCGGCACCCCTTCCGGCTGCGAGAATATCAGCGGCGCGTTTGCCTCTAACCGGACATTCGA
CATCACAGTGTCCGGCCTGGGCACAGCCCCCAAGACCCTGATCATCACCAGAACCGCCACAAACGCCA
CAACAACAACCCACAAGGTAATCTTTTCCAAGGCCCCAGAGTCTACCACAACATCCCCCACCCTGAAT
ACCACAGGCTTCGCCGACCCTAATACCACAACCGGCCTGCCTTCTAGCACCCACGTACCAACAAACCT
GACCGCCCCAGCCTCCACAGGCCCAACCGTGTCCACAGCCGACGTGACCTCCCCTACCCCCGCCGGCA
CCACATCCGGCGCGAGCCCAGTGACCCCATCCCCTTCCCCCAGGGATAATGGCACAGAGAGCAAGGCC
CCCGATATGACATCCTCTACAAGCCCTGTGACCACCCCTACCCCCAACGCCACCAGCCCCACCCCAGC
CGTGACCACCCCAACACCCAACGCCACCTCCCCTACACCTGCCGTGACAACCCCAACACCTAATGCCA
CATCCCCAACACTGGGCAAGACCTCCCCAACATCCGCCGTGACAACACCAACCCCCAATGCCACCTCT
CCAACACTGGGCAAGACATCCCCTACCTCCGCCGTGACCACACCTACCCCAAACGCCACATCCCCCAC
ACTGGGCAAGACCAGCCCAACATCTGCCGTGACAACCCCAACCCCAAATGCCACAGGCCCCACCGTGG
GCGAGACAAGCCCTCAGGCCAATGCCACAAATCACACCCTGGGCGGCACCTCCCCTACACCCGTGGTG
ACATCTCAGCCCAAGAATGCCACATCTGCCGTGACAACAGGCCAGCACAATATCACATCCTCTAGCAC
ATCCTCCATGTCCCTGCGCCCCTCTTCCATCCCAGAGACACTGAGCCCAAGCACCTCCGACAACTCCA
CATCTCACATGCCACTGCTGACCTCTGCCCACCCCACAGGCGGCGAGAACATCACCCAGGTGACACCT
GCCTCCATCAGCACCCACCACGTATCTACCAGCTCCCCTGCCCCAAGACCAGGCACAACAAGCCAGGC
CTCCGGCCCAGGCAATTCTTCCACATCTACCAAGCCAGGCGAGGTGAACGTGACCAAGGGCACCCCCC
CACAGAATGCCACCTCCCCTCAGGCCCCTTCCGGCCAGAAGACAGCCGTGCCAACCGTGACATCCACC
GGCGGCAAGGCCAATTCTACAACAGGCGGCAAGCACACCACCGGCCACGGCGCGAGAACCAGCACCGA
GCCTACAACCGACTACGGCGGCGATTCCACAACCCCAAGACCCAGATACAACGCCACCACCTACCTGC
CCCCTTCCACAAGCTCTAAGCTGAGACCAAGATGGACATTCACATCCCCCCCAGTGACCACAGCCCAG
GCCACAGTGCCCGTGCCCCCCACCAGCCAGCCACGGTTCTCCTAA
SEQ ID NO: 11 is a MMLV-Gag/EBV gp350 Codon Optimized Nucleotide Sequence
ATGGGCCAGACAGTGACCACCCCTCTGTCCCTGACCCTGGGCCACTGGAAGGACGTGGAAAGAATCGC
CCACAACCAGTCTGTTGACGTGAAGAAAAGAAGATGGGTGACCTTCTGCTCTGCCGAGTGGCCTACCT
TCAACGTTGGATGGCCCAGAGATGGCACCTTTAACAGAGACCTGATCACCCAGGTGAAGATCAAGGTG
TTCAGCCCTGGCCCCCATGGCCACCCTGATCAGGTGCCTTACATCGTGACCTGGGAGGCCCTGGCCTT
CGACCCCCCTCCATGGGTCAAACCTTTCGTGCACCCCAAGCCTCCACCCCCTCTGCCACCTAGCGCCC
CCTCCCTGCCCCTGGAGCCCCCGCGGAGCACACCCCCAAGATCCAGCCTCTATCCTGCCCTGACCCCG
TCCCTGGGAGCCAAGCCGAAGCCTCAGGTGCTGTCCGACAGCGGCGGACCACTGATCGACCTGCTGAC
GGAAGACCCTCCTCCTTACAGAGACCCTAGACCTCCTCCTTCTGATAGAGACGGCAACGGCGGCGAGG
CCACGCCCGCCGGTGAAGCCCCTGATCCCTCTCCCATGGCCAGCAGACTCAGAGGCCGGAGAGAACCC
CCTGTCGCCGATTCCACAACATCCCAAGCCTTCCCCCTGCGGGCCGGCGGCAACGGCCAACTGCAGTA
CTGGCCCTTTTCCAGCAGCGACCTGTACAACTGGAAGAACAATAATCCTAGTTTTTCTGAGGACCCTG
GCAAACTGACAGCTCTGATCGAGAGTGTGCTGATTACACACCAGCCCACCTGGGATGATTGCCAGCAA
CTGCTGGGCACCCTGCTGACAGGCGAGGAAAAGCAGAGAGTGCTGCTGGAGGCCAGAAAGGCCGTGCG
GGGCGATGATGGCCGGCCTACCCAACTGCCTAATGAGGTGGACGCCGCCTTTCCTCTGGAGAGACCTG
ATTGGGACTACACAACCCAAGCCGGACGAAACCATCTGGTCCACTACCGGCAACTGCTGCTGGCCGGC
CTGCAGAACGCGGGAAGAAGCCCCACCAACCTGGCTAAAGTGAAAGGCATAACGCAAGGGCCTAATGA
GTCACCCTCCGCCTTTCTGGAACGGCTGAAGGAAGCTTATAGGCGGTACACCCCCTACGACCCTGAGG
ACCCTGGCCAGGAGACCAACGTGAGCATGAGCTTCATCTGGCAGAGCGCCCCTGACATAGGAAGGAAG
CTGGAGCGGCTGGAGGATCTGAAGAACAAGACACTGGGCGATCTAGTGAGAGAGGCTGAGAAGATCTT
CAACAAGAGAGAGACCCCTGAGGAGAGAGAAGAACGGATCAGAAGAGAGACAGAGGAAAAGGAAGAAA
GAAGAAGAACAGAGGACGAGCAGAAGGAAAAAGAGAGAGACAGAAGACGCCACAGAGAGATGAGCAAG
CTGCTGGCCACCGTAGTTTCGGGACAGAAACAGGACCGGCAGGGCGGAGAAAGACGGCGGAGCCAACT
GGACCGGGACCAGTGCGCCTACTGCAAGGAGAAGGGCCACTGGGCCAAGGACTGTCCTAAAAAGCCAA
GAGGCCCTCGGGGACCTAGACCTCAAACAAGTCTGCTGACCCTGGACGACGACCCAGGCTTCTTCAAC
GTGGAAATCCCTGAGTTCCCTTTCTACCCTACCTGTAATGTGTGCACCGCCGACGTGAACGTCACCAT
CAACTTCGACGTGGGAGGCAAGAAGCACCAACTGGACTTGGACTTCGGCCAACTGACCCCCCACACCA
AGGCCGTGTACCAGCCTAGAGGCGCTTTCGGCGGCAGCGAGAACGCCACCAACCTGTTCCTGCTGGAG
CTGCTGGGAGCCGGCGAGCTGGCCCTGACCATGAGAAGCAAAAAGCTTCCTATCAACGTGACGACCGG
CGAAGAGCAGCAGGTGAGCCTGGAAAGCGTGGACGTCTACTTCCAGGATGTGTTCGGCACCATGTGGT
GCCACCACGCCGAGATGCAGAACCCTGTGTACCTGATCCCTGAGACCGTGCCCTACATCAAGTGGGAC
AACTGCAACTCCACAAACATCACCGCTGTGGTGAGGGCCCAGGGCCTGGACGTGACCCTGCCCCTGAG
CCTGCCTACTTCTGCCCAAGACAGCAACTTCTCAGTGAAGACCCAAATGCTGGGAAACGAGATCGACA
TCGAGTGCATCATGGAAGATGGCGAGATTAGTCAGGTGCTGCCTGGCGACAACAAGTTCAACATAACA
TGCAGCGGATACGAGAGCCATGTGCCTAGTGGGGGTATCCTGACTTCAACTAGCCCTGTGGCCACTCC
GATCCCCGGAACCGGCTACGCCTATTCTCTGAGACTTACACCAAGACCTGTGAGCAGATTCCTGGGCA
ACAACAGCATCCTGTACGTGTTCTACAGCGGCAATGGCCCCAAGGCGTCCGGCGGCGATTACTGTATC
CAGAGCAACATCGTGTTCAGCGATGAGATCCCTGCCAGCCAAGATATGCCCACCAACACCACCGACAT
CACATACGTGGGCGATAACGCCACCTACAGCGTGCCTATGGTGACCAGCGAGGACGCCAACTCTCCTA
ATGTGACCGTGACAGCCTTCTGGGCCTGGCCTAACAACACCGAGACGGATTTCAAGTGCAAGTGGACA
CTAACATCTGGAACCCCTAGCGGTTGTGAGAACATCAGCGGGGCCTTCGCCAGCAACAGAACCTTTGA
CATCACCGTCTCAGGTCTGGGCACCGCCCCAAAGACGCTGATCATCACCAGAACAGCCACCAACGCTA
CCACCACAACACATAAGGTGATTTTCAGCAAAGCCCCGGAATCTACCACCACCTCTCCCACCCTGAAC
ACGACAGGGTTTGCCGATCCCAATACTACCACCGGCCTGCCATCTAGCACCCATGTGCCTACCAACCT
GACAGCCCCAGCAAGCACCGGGCCAACGGTGTCGACCGCCGACGTGACCAGCCCTACGCCAGCCGGCA
CAACCTCTGGAGCCAGCCCAGTGACCCCTAGCCCTAGCCCTCGGGACAACGGCACAGAAAGCAAGGCC
CCCGACATGACCAGCAGCACCAGCCCCGTGACCACCCCTACACCCAACGCTACATCTCCCACACCAGC
CGTGACAACACCCACACCCAATGCTACCAGCCCTACCCCTGCCGTGACCACCCCCACCCCAAATGCCA
CCTCTCCTACCCTCGGCAAGACCAGCCCCACCTCTGCTGTTACCACCCCGACACCTAATGCCACTTCA
CCTACACTGGGCAAGACCAGCCCTACCAGCGCCGTTACAACTCCTACCCCCAACGCCACCAGCCCGAC
TCTGGGCAAAACTAGCCCCACCAGTGCCGTGACTACACCGACCCCGAATGCCACCGGCCCTACCGTTG
GAGAAACCAGCCCTCAGGCTAACGCGACCAACCACACACTGGGCGGCACAAGCCCTACCCCAGTGGTG
ACATCCCAGCCCAAGAACGCCACATCTGCGGTGACCACCGGCCAGCACAACATCACCTCCAGCAGCAC
CTCCAGCATGAGCCTCAGACCAAGCTCCATCCCTGAGACACTCAGCCCTAGCACAAGCGACAACTCTA
CCTCACACATGCCTCTGCTGACCAGCGCCCACCCTACCGGTGGCGAGAACATCACCCAGGTGACCCCA
GCTTCGATCAGCACGCACCATGTGAGCACATCTTCACCTGCTCCTAGACCTGGCACCACATCCCAGGC
CTCTGGCCCTGGCAACAGCAGCACCTCTACCAAGCCTGGCGAAGTGAACGTAACAAAGGGCACACCTC
CCCAGAACGCTACCTCGCCCCAAGCTCCCAGCGGGCAGAAGACCGCGGTTCCAACAGTGACTTCTACA
GGAGGCAAGGCCAATAGCACCACAGGCGGAAAGCACACCACCGGCCACGGCGCACGGACATCTACAGA
ACCTACAACTGACTACGGCGGCGACAGCACCACCCCCAGACCTAGATACAACGCCACCACCTACCTGC
CTCCATCTACAAGCAGCAAGCTGAGACCCAGATGGACCTTCACGTCCCCTCCTGTGACCACCGCTCAG
GCTACAGTCCCTGTGCCCCCTACCAGCCAGCCTCGCTTCAGCTGA
SEQ ID NO: 12 is a MMLV-Gag/EBV gp350 mRNA Sequence
AUGGGCCAGACAGUGACCACCCCUCUGUCCCUGACCCUGGGCCACUGGAAGGACGUGGAAAGAAUCGC
CCACAACCAGUCUGUUGACGUGAAGAAAAGAAGAUGGGUGACCUUCUGCUCUGCCGAGUGGCCUACCU
UCAACGUUGGAUGGCCCAGAGAUGGCACCUUUAACAGAGACCUGAUCACCCAGGUGAAGAUCAAGGUG
UUCAGCCCUGGCCCCCAUGGCCACCCUGAUCAGGUGCCUUACAUCGUGACCUGGGAGGCCCUGGCCUU
CGACCCCCCUCCAUGGGUCAAACCUUUCGUGCACCCCAAGCCUCCACCCCCUCUGCCACCUAGCGCCC
CCUCCCUGCCCCUGGAGCCCCCGCGGAGCACACCCCCAAGAUCCAGCCUCUAUCCUGCCCUGACCCCG
UCCCUGGGAGCCAAGCCGAAGCCUCAGGUGCUGUCCGACAGCGGCGGACCACUGAUCGACCUGCUGAC
GGAAGACCCUCCUCCUUACAGAGACCCUAGACCUCCUCCUUCUGAUAGAGACGGCAACGGCGGCGAGG
CCACGCCCGCCGGUGAAGCCCCUGAUCCCUCUCCCAUGGCCAGCAGACUCAGAGGCCGGAGAGAACCC
CCUGUCGCCGAUUCCACAACAUCCCAAGCCUUCCCCCUGCGGGCCGGCGGCAACGGCCAACUGCAGUA
CUGGCCCUUUUCCAGCAGCGACCUGUACAACUGGAAGAACAAUAAUCCUAGUUUUUCUGAGGACCCUG
GCAAACUGACAGCUCUGAUCGAGAGUGUGCUGAUUACACACCAGCCCACCUGGGAUGAUUGCCAGCAA
CUGCUGGGCACCCUGCUGACAGGCGAGGAAAAGCAGAGAGUGCUGCUGGAGGCCAGAAAGGCCGUGCG
GGGCGAUGAUGGCCGGCCUACCCAACUGCCUAAUGAGGUGGACGCCGCCUUUCCUCUGGAGAGACCUG
AUUGGGACUACACAACCCAAGCCGGACGAAACCAUCUGGUCCACUACCGGCAACUGCUGCUGGCCGGC
CUGCAGAACGCGGGAAGAAGCCCCACCAACCUGGCUAAAGUGAAAGGCAUAACGCAAGGGCCUAAUGA
GUCACCCUCCGCCUUUCUGGAACGGCUGAAGGAAGCUUAUAGGCGGUACACCCCCUACGACCCUGAGG
ACCCUGGCCAGGAGACCAACGUGAGCAUGAGCUUCAUCUGGCAGAGCGCCCCUGACAUAGGAAGGAAG
CUGGAGCGGCUGGAGGAUCUGAAGAACAAGACACUGGGCGAUCUAGUGAGAGAGGCUGAGAAGAUCUU
CAACAAGAGAGAGACCCCUGAGGAGAGAGAAGAACGGAUCAGAAGAGAGACAGAGGAAAAGGAAGAAA
GAAGAAGAACAGAGGACGAGCAGAAGGAAAAAGAGAGAGACAGAAGACGCCACAGAGAGAUGAGCAAG
CUGCUGGCCACCGUAGUUUCGGGACAGAAACAGGACCGGCAGGGCGGAGAAAGACGGCGGAGCCAACU
GGACCGGGACCAGUGCGCCUACUGCAAGGAGAAGGGCCACUGGGCCAAGGACUGUCCUAAAAAGCCAA
GAGGCCCUCGGGGACCUAGACCUCAAACAAGUCUGCUGACCCUGGACGACGACCCAGGCUUCUUCAAC
GUGGAAAUCCCUGAGUUCCCUUUCUACCCUACCUGUAAUGUGUGCACCGCCGACGUGAACGUCACCAU
CAACUUCGACGUGGGAGGCAAGAAGCACCAACUGGACUUGGACUUCGGCCAACUGACCCCCCACACCA
AGGCCGUGUACCAGCCUAGAGGCGCUUUCGGCGGCAGCGAGAACGCCACCAACCUGUUCCUGCUGGAG
CUGCUGGGAGCCGGCGAGCUGGCCCUGACCAUGAGAAGCAAAAAGCUUCCUAUCAACGUGACGACCGG
CGAAGAGCAGCAGGUGAGCCUGGAAAGCGUGGACGUCUACUUCCAGGAUGUGUUCGGCACCAUGUGGU
GCCACCACGCCGAGAUGCAGAACCCUGUGUACCUGAUCCCUGAGACCGUGCCCUACAUCAAGUGGGAC
AACUGCAACUCCACAAACAUCACCGCUGUGGUGAGGGCCCAGGGCCUGGACGUGACCCUGCCCCUGAG
CCUGCCUACUUCUGCCCAAGACAGCAACUUCUCAGUGAAGACCCAAAUGCUGGGAAACGAGAUCGACA
UCGAGUGCAUCAUGGAAGAUGGCGAGAUUAGUCAGGUGCUGCCUGGCGACAACAAGUUCAACAUAACA
UGCAGCGGAUACGAGAGCCAUGUGCCUAGUGGGGGUAUCCUGACUUCAACUAGCCCUGUGGCCACUCC
GAUCCCCGGAACCGGCUACGCCUAUUCUCUGAGACUUACACCAAGACCUGUGAGCAGAUUCCUGGGCA
ACAACAGCAUCCUGUACGUGUUCUACAGCGGCAAUGGCCCCAAGGCGUCCGGCGGCGAUUACUGUAUC
CAGAGCAACAUCGUGUUCAGCGAUGAGAUCCCUGCCAGCCAAGAUAUGCCCACCAACACCACCGACAU
CACAUACGUGGGCGAUAACGCCACCUACAGCGUGCCUAUGGUGACCAGCGAGGACGCCAACUCUCCUA
AUGUGACCGUGACAGCCUUCUGGGCCUGGCCUAACAACACCGAGACGGAUUUCAAGUGCAAGUGGACA
CUAACAUCUGGAACCCCUAGCGGUUGUGAGAACAUCAGCGGGGCCUUCGCCAGCAACAGAACCUUUGA
CAUCACCGUCUCAGGUCUGGGCACCGCCCCAAAGACGCUGAUCAUCACCAGAACAGCCACCAACGCUA
CCACCACAACACAUAAGGUGAUUUUCAGCAAAGCCCCGGAAUCUACCACCACCUCUCCCACCCUGAAC
ACGACAGGGUUUGCCGAUCCCAAUACUACCACCGGCCUGCCAUCUAGCACCCAUGUGCCUACCAACCU
GACAGCCCCAGCAAGCACCGGGCCAACGGUGUCGACCGCCGACGUGACCAGCCCUACGCCAGCCGGCA
CAACCUCUGGAGCCAGCCCAGUGACCCCUAGCCCUAGCCCUCGGGACAACGGCACAGAAAGCAAGGCC
CCCGACAUGACCAGCAGCACCAGCCCCGUGACCACCCCUACACCCAACGCUACAUCUCCCACACCAGC
CGUGACAACACCCACACCCAAUGCUACCAGCCCUACCCCUGCCGUGACCACCCCCACCCCAAAUGCCA
CCUCUCCUACCCUCGGCAAGACCAGCCCCACCUCUGCUGUUACCACCCCGACACCUAAUGCCACUUCA
CCUACACUGGGCAAGACCAGCCCUACCAGCGCCGUUACAACUCCUACCCCCAACGCCACCAGCCCGAC
UCUGGGCAAAACUAGCCCCACCAGUGCCGUGACUACACCGACCCCGAAUGCCACCGGCCCUACCGUUG
GAGAAACCAGCCCUCAGGCUAACGCGACCAACCACACACUGGGCGGCACAAGCCCUACCCCAGUGGUG
ACAUCCCAGCCCAAGAACGCCACAUCUGCGGUGACCACCGGCCAGCACAACAUCACCUCCAGCAGCAC
CUCCAGCAUGAGCCUCAGACCAAGCUCCAUCCCUGAGACACUCAGCCCUAGCACAAGCGACAACUCUA
CCUCACACAUGCCUCUGCUGACCAGCGCCCACCCUACCGGUGGCGAGAACAUCACCCAGGUGACCCCA
GCUUCGAUCAGCACGCACCAUGUGAGCACAUCUUCACCUGCUCCUAGACCUGGCACCACAUCCCAGGC
CUCUGGCCCUGGCAACAGCAGCACCUCUACCAAGCCUGGCGAAGUGAACGUAACAAAGGGCACACCUC
CCCAGAACGCUACCUCGCCCCAAGCUCCCAGCGGGCAGAAGACCGCGGUUCCAACAGUGACUUCUACA
GGAGGCAAGGCCAAUAGCACCACAGGCGGAAAGCACACCACCGGCCACGGCGCACGGACAUCUACAGA
ACCUACAACUGACUACGGCGGCGACAGCACCACCCCCAGACCUAGAUACAACGCCACCACCUACCUGC
CUCCAUCUACAAGCAGCAAGCUGAGACCCAGAUGGACCUUCACGUCCCCUCCUGUGACCACCGCUCAG
GCUACAGUCCCUGUGCCCCCUACCAGCCAGCCUCGCUUCAGCUGA

In some embodiments, an envelope polypeptide includes a transmembrane domain and cytoplasmic domain found in nature in vesicular stomatitis virus (VSV). As is known in the art, polypeptides sometimes have transmembrane, cytoplasmic, and/or extracellular domains. In general, a “transmembrane domain”, as used herein, refers to a domain that has an attribute of being present in the membrane (e.g., spanning a portion or all of a cellular membrane). As will be appreciated, it is not required that every amino acid in a transmembrane domain be present in the membrane. For example, in some embodiments, a transmembrane domain is characterized in that a designated stretch or portion of a protein is substantially located in the membrane. In general, a “cytoplasmic domain”, as used herein, refers to a domain that has an attribute of being present in the cytoplasm. As will be appreciated, it is not required that every amino acid in a cytoplasmic domain be present in the cytoplasm. For example, in some embodiments, a cytoplasmic domain is characterized in that a designated stretch or portion of a protein is substantially located in the cytoplasm.

As is well known in the art, amino acid or nucleic acid sequences may be analyzed using a variety of algorithms to predict protein subcellular localization (e.g., transmembrane localization or cytoplasmic localization). Exemplary such programs include psort (PSORT.org), Prosite (prosite.expasy.org), PROTTER (https://wlab.ethz.ch/protter/start/), UNIPROT (uniprot.org), among others.

The transmembrane domain of VSV-G functions to target the viral glycoprotein to the cell membrane (Compton T et al., 1989 Proc Natl Acad Sci USA 86:4112-4116). Swapping the transmembrane and cytoplasmic domains of VSV-G for the transmembrane and cytoplasmic domains of another protein has been used to direct a protein to the cell membrane when the native protein does not naturally do this or requires accessory co-expressed proteins to accomplish this (Garrone P et al., 2011 Sci Transl Med 3 (94): 94ra71).

SEQ ID NO: 13 is an EBV gp350 Glycoprotein, Amino Acid Sequence
MEAALLVCQYTIQSLIHLTGEDPGFENVEIPEFPFYPTCNVCTADVNVTINFDVGGKKHQLDLDFGQL
TPHTKAVYQPRGAFGGSENATNLFLLELLGAGELALTMRSKKLPINVTTGEEQQVSLESVDVYFQDVE
GTMWCHHAEMQNPVYLIPETVPYIKWDNCNSTNITAVVRAQGLDVTLPLSLPTSAQDSNESVKTQMLG
NEIDIECIMEDGEISQVLPGDNKFNITCSGYESHVPSGGILTSTSPVATPIPGTGYAYSLRLTPRPVS
RFLGNNSILYVFYSGNGPKASGGDYCIQSNIVESDEIPASQDMPTNTTDITYVGDNATYSVPMVTSED
ANSPNVTVTAFWAWPNNTETDFKCKWTLTSGTPSGCENISGAFASNRTFDITVSGLGTAPKTLIITRT
ATNATTTTHKVIFSKAPESTTTSPTLNTTGFADPNTTTGLPSSTHVPTNLTAPASTGPTVSTADVTSP
TPAGTTSGASPVTPSPSPRDNGTESKAPDMTSSTSPVTTPTPNATSPTPAVTTPTPNATSPTPAVTTP
TPNATSPTLGKTSPTSAVTTPTPNATSPTLGKTSPTSAVTTPTPNATSPTLGKTSPTSAVTTPTPNAT
GPTVGETSPQANATNHTLGGTSPTPVVTSQPKNATSAVTTGQHNITSSSTSSMSLRPSSIPETLSPST
SDNSTSHMPLLTSAHPTGGENITQVTPASISTHHVSTSSPAPRPGTTSQASGPGNSSTSTKPGEVNVT
KGTPPQNATSPQAPSGQKTAVPTVTSTGGKANSTTGGKHTTGHGARTSTEPTTDYGGDSTTPRPRYNA
TTYLPPSTSSKLRPRWTFTSPPVTTAQATVPVPPTSQPRESNLSMLVLQWASLAVLTLLLLLVMADCA
FRRNLSTSHTYTTPPYDDAETYV
SEQ ID NO: 14 is an EBV gp350 Glycoprotein, Nucleotide Sequence (BLLF1
gene from strain B95.8: Genbank Ref: NC_007605.1 with 3 point mutations G601C,
T1483A, A2015T resulting in aa changes E201Q, W495R, N672I). Note that mutations
were designed to better fit with highly represented isolates found in NPC and GC,
that are closer tothe Asian GD1 strain:
ATGGAGGCAGCCTTGCTTGTGTGTCAGTACACCATCCAGAGCCTGATCCATCTCACGGGTGAAGATCC
TGGTTTTTTCAATGTTGAGATTCCGGAATTCCCATTTTACCCCACATGCAATGTTTGCACGGCAGATG
TCAATGTAACTATCAATTTCGATGTCGGGGGCAAAAAGCATCAACTTGATCTTGACTTTGGCCAGCTG
ACACCCCATACGAAGGCTGTCTACCAACCTCGAGGTGCATTTGGTGGCTCAGAAAATGCCACCAATCT
CTTTCTACTGGAGCTCCTTGGTGCAGGAGAATTGGCTCTAACTATGCGGTCTAAGAAGCTTCCAATTA
ACGTCACCACCGGAGAGGAGCAACAAGTAAGCCTGGAATCTGTAGATGTCTACTTTCAAGATGTGTTT
GGAACCATGTGGTGCCACCATGCAGAAATGCAAAACCCCGTGTACCTGATACCAGAAACAGTGCCATA
CATAAAGTGGGATAACTGTAATTCTACCAATATAACGGCAGTAGTGAGGGCACAGGGGCTGGATGTCA
CGCTACCCTTAAGTTTGCCAACGTCAGCTCAAGACTCGAATTTCAGCGTAAAAACACAAATGCTCGGT
AATGAGATAGATATTGAGTGTATTATGGAGGATGGCGAAATTTCACAAGTTCTGCCCGGAGACAACAA
ATTTAACATCACCTGCAGTGGATACGAGAGCCATGTTCCCAGCGGCGGAATTCTCACATCAACGAGTC
CCGTGGCCACCCCAATACCTGGTACAGGGTATGCATACAGCCTGCGTCTGACACCACGTCCAGTGTCA
CGATTTCTTGGCAATAACAGTATCCTGTACGTGTTTTACTCTGGGAATGGACCGAAGGCGAGCGGGGG
AGATTACTGCATTCAGTCCAACATTGTGTTCTCTGATGAGATTCCAGCTTCACAGGACATGCCGACAA
ACACCACAGACATCACATATGTGGGTGACAATGCTACCTATTCAGTGCCAATGGTCACTTCTGAGGAC
GCAAACTCGCCAAATGTTACAGTGACTGCCTTTTGGGCCTGGCCAAACAACACTGAAACTGACTTTAA
GTGCAAATGGACTCTCACCTCGGGGACACCTTCGGGTTGTGAAAATATTTCTGGTGCATTTGCGAGCA
ATCGGACATTTGACATTACTGTCTCGGGTCTTGGCACGGCCCCCAAGACACTCATTATCACACGAACG
GCTACCAATGCCACCACAACAACCCACAAGGTTATATTCTCCAAGGCACCCGAGAGCACCACCACCTC
CCCTACCTTGAATACAACTGGATTTGCTGATCCCAATACAACGACAGGTCTACCCAGCTCTACTCACG
TGCCTACCAACCTCACCGCACCTGCAAGCACAGGCCCCACTGTATCCACCGCGGATGTCACCAGCCCA
ACACCAGCCGGCACAACGTCAGGCGCATCACCGGTGACACCAAGTCCATCTCCAAGGGACAACGGCAC
AGAAAGTAAGGCCCCCGACATGACCAGCTCCACCTCACCAGTGACTACCCCAACCCCAAATGCCACCA
GCCCCACCCCAGCAGTGACTACCCCAACCCCAAATGCCACCAGCCCCACCCCAGCAGTGACTACCCCA
ACCCCAAATGCCACCAGCCCCACCTTGGGAAAAACAAGTCCTACCTCAGCAGTGACTACCCCAACCCC
AAATGCCACCAGCCCCACCTTGGGAAAAACAAGCCCCACCTCAGCAGTGACTACCCCAACCCCAAATG
CCACCAGCCCCACCTTGGGAAAAACAAGCCCCACCTCAGCAGTGACTACCCCAACCCCAAATGCCACC
GGCCCTACTGTGGGAGAAACAAGTCCACAGGCAAATGCCACCAACCACACCTTAGGAGGAACAAGTCC
CACCCCAGTAGTTACCAGCCAACCAAAAAATGCAACCAGTGCTGTTACCACAGGCCAACATAACATAA
CTTCAAGTTCAACCTCTTCCATGTCACTGAGACCCAGTTCAATCCCAGAGACACTCAGCCCCTCCACC
AGTGACAATTCAACGTCACATATGCCTTTACTAACCTCCGCTCACCCAACAGGTGGTGAAAATATAAC
ACAGGTGACACCAGCCTCTATCAGCACACATCATGTGTCCACCAGTTCGCCAGCACCCCGCCCAGGCA
CCACCAGCCAAGCGTCAGGCCCTGGAAACAGTTCCACATCCACAAAACCGGGGGAGGTTAATGTCACC
AAAGGCACGCCCCCCCAAAATGCAACGTCGCCCCAGGCCCCCAGTGGCCAAAAGACGGCGGTTCCCAC
GGTCACCTCAACAGGTGGAAAGGCCAATTCTACCACCGGTGGAAAGCACACCACAGGACATGGAGCCC
GGACAAGTACAGAGCCCACCACAGATTACGGCGGTGATTCAACTACGCCAAGACCGAGATACAATGCG
ACCACCTATCTACCTCCCAGCACTTCTAGCAAACTGCGGCCCCGCTGGACTTTTACGAGCCCACCGGT
TACCACAGCCCAAGCCACCGTGCCAGTCCCGCCAACGTCCCAGCCCAGATTCTCAAACCTCTCCATGC
TAGTACTGCAGTGGGCCTCTCTGGCTGTGCTGACCCTTCTGCTGCTGCTGGTCATGGCGGACTGCGCC
TTTAGGCGTAACTTGTCTACATCCCATACCTACACCACCCCACCATATGATGACGCCGAGACCTATGT
ATAA
SEQ ID NO: 15 is an EBV gp350 Glycoprotein, Nucleotide Sequence, Codon
Optimized For Expression In Human Cell
ATGGAGGCCGCTCTGCTGGTCTGCCAGTACACTATTCAGTCTCTGATTCACCTGACCGGGGAAGATCC
TGGGTTTTTCAATGTGGAGATCCCTGAGTTTCCATTCTACCCAACATGCAACGTGTGCACCGCCGACG
TGAACGTGACCATCAATTTTGATGTGGGCGGCAAGAAGCACCAGCTAGACCTGGATTTTGGCCAGCTA
ACACCTCACACCAAGGCCGTGTATCAGCCACGCGGCGCGTTTGGCGGCAGCGAGAACGCCACAAACCT
GTTTCTGCTGGAGCTGCTGGGCGCGGGCGAGCTGGCCCTGACCATGAGAAGCAAGAAGCTGCCAATCA
ACGTGACCACCGGCGAGGAGCAGCAGGTGTCCCTGGAGTCCGTGGACGTGTACTTCCAGGACGTGTTC
GGCACAATGTGGTGCCACCACGCCGAGATGCAGAACCCAGTGTACCTGATCCCAGAGACCGTGCCATA
CATCAAGTGGGACAACTGCAACTCCACCAATATCACAGCCGTGGTGCGGGCCCAGGGCCTGGATGTGA
CACTGCCTCTGAGCCTGCCTACATCTGCCCAGGACAGCAACTTTTCCGTGAAGACCCAGATGCTGGGC
AACGAGATCGACATCGAGTGCATCATGGAGGATGGCGAGATCTCCCAGGTGCTGCCTGGCGACAATAA
GTTCAACATCACATGTAGCGGCTACGAGAGCCACGTACCTTCCGGCGGCATCCTGACCTCTACAAGCC
CAGTGGCCACCCCAATCCCCGGCACCGGCTATGCCTACTCTCTGAGGCTGACACCTCGGCCCGTGAGC
AGATTCCTGGGCAACAACTCCATCCTGTACGTGTTCTACTCCGGCAATGGCCCTAAGGCCAGCGGCGG
CGACTACTGTATCCAGAGCAACATCGTGTTTTCCGACGAGATCCCTGCCTCTCAGGATATGCCCACCA
ATACCACCGACATCACATACGTGGGCGACAATGCCACATATAGCGTGCCAATGGTGACCAGCGAGGAC
GCCAATAGCCCTAACGTGACAGTGACCGCCTTTTGGGCCTGGCCCAACAATACAGAGACCGATTTCAA
GTGTAAGTGGACCCTGACCAGCGGCACCCCTTCCGGCTGCGAGAATATCAGCGGCGCGTTTGCCTCTA
ACCGGACATTCGACATCACAGTGTCCGGCCTGGGCACAGCCCCCAAGACCCTGATCATCACCAGAACC
GCCACAAACGCCACAACAACAACCCACAAGGTAATCTTTTCCAAGGCCCCAGAGTCTACCACAACATC
CCCCACCCTGAATACCACAGGCTTCGCCGACCCTAATACCACAACCGGCCTGCCTTCTAGCACCCACG
TACCAACAAACCTGACCGCCCCAGCCTCCACAGGCCCAACCGTGTCCACAGCCGACGTGACCTCCCCT
ACCCCCGCCGGCACCACATCCGGCGCGAGCCCAGTGACCCCATCCCCTTCCCCCAGGGATAATGGCAC
AGAGAGCAAGGCCCCCGATATGACATCCTCTACAAGCCCTGTGACCACCCCTACCCCCAACGCCACCA
GCCCCACCCCAGCCGTGACCACCCCAACACCCAACGCCACCTCCCCTACACCTGCCGTGACAACCCCA
ACACCTAATGCCACATCCCCAACACTGGGCAAGACCTCCCCAACATCCGCCGTGACAACACCAACCCC
CAATGCCACCTCTCCAACACTGGGCAAGACATCCCCTACCTCCGCCGTGACCACACCTACCCCAAACG
CCACATCCCCCACACTGGGCAAGACCAGCCCAACATCTGCCGTGACAACCCCAACCCCAAATGCCACA
GGCCCCACCGTGGGCGAGACAAGCCCTCAGGCCAATGCCACAAATCACACCCTGGGCGGCACCTCCCC
TACACCCGTGGTGACATCTCAGCCCAAGAATGCCACATCTGCCGTGACAACAGGCCAGCACAATATCA
CATCCTCTAGCACATCCTCCATGTCCCTGCGCCCCTCTTCCATCCCAGAGACACTGAGCCCAAGCACC
TCCGACAACTCCACATCTCACATGCCACTGCTGACCTCTGCCCACCCCACAGGCGGCGAGAACATCAC
CCAGGTGACACCTGCCTCCATCAGCACCCACCACGTATCTACCAGCTCCCCTGCCCCAAGACCAGGCA
CAACAAGCCAGGCCTCCGGCCCAGGCAATTCTTCCACATCTACCAAGCCAGGCGAGGTGAACGTGACC
AAGGGCACCCCCCCACAGAATGCCACCTCCCCTCAGGCCCCTTCCGGCCAGAAGACAGCCGTGCCAAC
CGTGACATCCACCGGCGGCAAGGCCAATTCTACAACAGGCGGCAAGCACACCACCGGCCACGGCGCGA
GAACCAGCACCGAGCCTACAACCGACTACGGCGGCGATTCCACAACCCCAAGACCCAGATACAACGCC
ACCACCTACCTGCCCCCTTCCACAAGCTCTAAGCTGAGACCAAGATGGACATTCACATCCCCCCCAGT
GACCACAGCCCAGGCCACAGTGCCCGTGCCCCCCACCAGCCAGCCACGGTTCTCCAACCTGTCTATGC
TGGTGCTGCAGTGGGCCTCTCTGGCCGTGCTGACACTGCTGCTGCTGCTGGTAATGGCCGATTGCGCC
TTCCGGCGGAATCTGTCAACAAGCCACACCTACACTACACCTCCTTACGATGATGCCGAAACTTATGT
CTGA
SEQ ID NO: 16 is an EBV gp350 Glycoprotein mRNA Sequence
AUGGAGGCCGCUCUGCUGGUCUGCCAGUACACUAUUCAGUCUCUGAUUCACCUGACCGGGGAAGAUCC
UGGGUUUUUCAAUGUGGAGAUCCCUGAGUUUCCAUUCUACCCAACAUGCAACGUGUGCACCGCCGACG
UGAACGUGACCAUCAAUUUUGAUGUGGGCGGCAAGAAGCACCAGCUAGACCUGGAUUUUGGCCAGCUA
ACACCUCACACCAAGGCCGUGUAUCAGCCACGCGGCGCGUUUGGCGGCAGCGAGAACGCCACAAACCU
GUUUCUGCUGGAGCUGCUGGGCGCGGGCGAGCUGGCCCUGACCAUGAGAAGCAAGAAGCUGCCAAUCA
ACGUGACCACCGGCGAGGAGCAGCAGGUGUCCCUGGAGUCCGUGGACGUGUACUUCCAGGACGUGUUC
GGCACAAUGUGGUGCCACCACGCCGAGAUGCAGAACCCAGUGUACCUGAUCCCAGAGACCGUGCCAUA
CAUCAAGUGGGACAACUGCAACUCCACCAAUAUCACAGCCGUGGUGCGGGCCCAGGGCCUGGAUGUGA
CACUGCCUCUGAGCCUGCCUACAUCUGCCCAGGACAGCAACUUUUCCGUGAAGACCCAGAUGCUGGGC
AACGAGAUCGACAUCGAGUGCAUCAUGGAGGAUGGCGAGAUCUCCCAGGUGCUGCCUGGCGACAAUAA
GUUCAACAUCACAUGUAGCGGCUACGAGAGCCACGUACCUUCCGGCGGCAUCCUGACCUCUACAAGCC
CAGUGGCCACCCCAAUCCCCGGCACCGGCUAUGCCUACUCUCUGAGGCUGACACCUCGGCCCGUGAGC
AGAUUCCUGGGCAACAACUCCAUCCUGUACGUGUUCUACUCCGGCAAUGGCCCUAAGGCCAGCGGCGG
CGACUACUGUAUCCAGAGCAACAUCGUGUUUUCCGACGAGAUCCCUGCCUCUCAGGAUAUGCCCACCA
AUACCACCGACAUCACAUACGUGGGCGACAAUGCCACAUAUAGCGUGCCAAUGGUGACCAGCGAGGAC
GCCAAUAGCCCUAACGUGACAGUGACCGCCUUUUGGGCCUGGCCCAACAAUACAGAGACCGAUUUCAA
GUGUAAGUGGACCCUGACCAGCGGCACCCCUUCCGGCUGCGAGAAUAUCAGCGGCGCGUUUGCCUCUA
ACCGGACAUUCGACAUCACAGUGUCCGGCCUGGGCACAGCCCCCAAGACCCUGAUCAUCACCAGAACC
GCCACAAACGCCACAACAACAACCCACAAGGUAAUCUUUUCCAAGGCCCCAGAGUCUACCACAACAUC
CCCCACCCUGAAUACCACAGGCUUCGCCGACCCUAAUACCACAACCGGCCUGCCUUCUAGCACCCACG
UACCAACAAACCUGACCGCCCCAGCCUCCACAGGCCCAACCGUGUCCACAGCCGACGUGACCUCCCCU
ACCCCCGCCGGCACCACAUCCGGCGCGAGCCCAGUGACCCCAUCCCCUUCCCCCAGGGAUAAUGGCAC
AGAGAGCAAGGCCCCCGAUAUGACAUCCUCUACAAGCCCUGUGACCACCCCUACCCCCAACGCCACCA
GCCCCACCCCAGCCGUGACCACCCCAACACCCAACGCCACCUCCCCUACACCUGCCGUGACAACCCCA
ACACCUAAUGCCACAUCCCCAACACUGGGCAAGACCUCCCCAACAUCCGCCGUGACAACACCAACCCC
CAAUGCCACCUCUCCAACACUGGGCAAGACAUCCCCUACCUCCGCCGUGACCACACCUACCCCAAACG
CCACAUCCCCCACACUGGGCAAGACCAGCCCAACAUCUGCCGUGACAACCCCAACCCCAAAUGCCACA
GGCCCCACCGUGGGCGAGACAAGCCCUCAGGCCAAUGCCACAAAUCACACCCUGGGCGGCACCUCCCC
UACACCCGUGGUGACAUCUCAGCCCAAGAAUGCCACAUCUGCCGUGACAACAGGCCAGCACAAUAUCA
CAUCCUCUAGCACAUCCUCCAUGUCCCUGCGCCCCUCUUCCAUCCCAGAGACACUGAGCCCAAGCACC
UCCGACAACUCCACAUCUCACAUGCCACUGCUGACCUCUGCCCACCCCACAGGCGGCGAGAACAUCAC
CCAGGUGACACCUGCCUCCAUCAGCACCCACCACGUAUCUACCAGCUCCCCUGCCCCAAGACCAGGCA
CAACAAGCCAGGCCUCCGGCCCAGGCAAUUCUUCCACAUCUACCAAGCCAGGCGAGGUGAACGUGACC
AAGGGCACCCCCCCACAGAAUGCCACCUCCCCUCAGGCCCCUUCCGGCCAGAAGACAGCCGUGCCAAC
CGUGACAUCCACCGGCGGCAAGGCCAAUUCUACAACAGGCGGCAAGCACACCACCGGCCACGGCGCGA
GAACCAGCACCGAGCCUACAACCGACUACGGCGGCGAUUCCACAACCCCAAGACCCAGAUACAACGCC
ACCACCUACCUGCCCCCUUCCACAAGCUCUAAGCUGAGACCAAGAUGGACAUUCACAUCCCCCCCAGU
GACCACAGCCCAGGCCACAGUGCCCGUGCCCCCCACCAGCCAGCCACGGUUCUCCAACCUGUCUAUGC
UGGUGCUGCAGUGGGCCUCUCUGGCCGUGCUGACACUGCUGCUGCUGCUGGUAAUGGCCGAUUGCGCC
UUCCGGCGGAAUCUGUCAACAAGCCACACCUACACUACACCUCCUUACGAUGAUGCCGAAACUUAUGU
CUGA
SEQ ID NO: 17 is a MERS-CoV Spike Glycoprotein, Amino Acid Sequence
MIHSVFLLMELLTPTESYVDVGPDSVKSACIEVDIQQTFFDKTWPRPIDVSKADGIIYPQGRTYSNIT
ITYQGLFPYQGDHGDMYVYSAGHATGTTPQKLFVANYSQDVKQFANGFVVRIGAAANSTGTVIISPST
SATIRKIYPAFMLGSSVGNFSDGKMGRFFNHTLVLLPDGCGTLLRAFYCILEPRSGNHCPAGNSYTSE
ATYHTPATDCSDGNYNRNASLNSFKEYFNLRNCTEMYTYNITEDEILEWFGITQTAQGVHLESSRYVD
LYGGNMFQFATLPVYDTIKYYSIIPHSIRSIQSDRKAWAAFYVYKLQPLTELLDESVDGYIRRAIDCG
FNDLSQLHCSYESFDVESGVYSVSSFEAKPSGSVVEQAEGVECDESPLLSGTPPQVYNFKRLVFTNCN
YNLTKLLSLESVNDFTCSQISPAAIASNCYSSLILDYFSYPLSMKSDLSVSSAGPISQFNYKQSFSNP
TCLILATVPHNLTTITKPLKYSYINKCSRLLSDDRTEVPQLVNANQYSPCVSIVPSTVWEDGDYYRKQ
LSPLEGGGWLVASGSTVAMTEQLQMGFGITVQYGTDTNSVCPKLEFANDTKIASQLGNCVEYSLYGVS
GRGVFQNCTAVGVRQQREVYDAYQNLVGYYSDDGNYYCLRACVSVPVSVIYDKETKTHATLEGSVACE
HISSTMSQYSRSTRSMLKRRDSTYGPLQTPVGCVLGLVNSSLFVEDCKLPLGQSLCALPDTPSTLTPR
SVRSVPGEMRLASIAFNHPIQVDQLNSSYFKLSIPTNFSFGVTQEYIQTTIQKVTVDCKQYVCNGFQK
CEQLLREYGQFCSKINQALHGANLRQDDSVRNLFASVKSSQSSPIIPGEGGDENLTLLEPVSISTGSR
SARSAIEDLLEDKVTIADPGYMQGYDDCMQQGPASARDLICAQYVAGYKVLPPLMDVNMEAAYTSSLL
GSIAGVGWTAGLSSFAAIPFAQSIFYRLNGVGITQQVLSENQKLIANKENQALGAMQTGFTTTNEAFQ
KVQDAVNNNAQALSKLASELSNTFGAISASIGDIIQRLDVLEQDAQIDRLINGRLTTLNAFVAQQLVR
SESAALSAQLAKDKVNECVKAQSKRSGFCGQGTHIVSFVVNAPNGLYFMHVGYYPSNHIEVVSAYGLC
DAANPTNCIAPVNGYFIKTNNTRIVDEWSYTGSSFYAPEPITSLNTKYVAPQVTYQNISTNLPPPLLG
NSTGIDFQDELDEFFKNVSTSIPNFGSLTQINTTLLDLTYEMLSLQQVVKALNESYIDLKELGNYTYY
NKWPWYIWLGFIAGLVALALCVFFILCCTGCGTNCMGKLKCNRCCDRYEEYDLEPHKVHVH
SEQ ID NO: 18 is a MERS-CoV Spike Glycoprotein, Nucleotide Sequence
(EMC/2012, Genbank Ref: JX869059.2)
ATGATACACTCAGTGTTTCTACTGATGTTCTTGTTAACACCTACAGAAAGTTACGTTGATGTAGGGCC
AGATTCTGTTAAGTCTGCTTGTATTGAGGTTGATATACAACAGACTTTCTTTGATAAAACTTGGCCTA
GGCCAATTGATGTTTCTAAGGCTGACGGTATTATATACCCTCAAGGCCGTACATATTCTAACATAACT
ATCACTTATCAAGGTCTTTTTCCCTATCAGGGAGACCATGGTGATATGTATGTTTACTCTGCAGGACA
TGCTACAGGCACAACTCCACAAAAGTTGTTTGTAGCTAACTATTCTCAGGACGTCAAACAGTTTGCTA
ATGGGTTTGTCGTCCGTATAGGAGCAGCTGCCAATTCCACTGGCACTGTTATTATTAGCCCATCTACC
AGCGCTACTATACGAAAAATTTACCCTGCTTTTATGCTGGGTTCTTCAGTTGGTAATTTCTCAGATGG
TAAAATGGGCCGCTTCTTCAATCATACTCTAGTTCTTTTGCCCGATGGATGTGGCACTTTACTTAGAG
CTTTTTATTGTATTCTAGAGCCTCGCTCTGGAAATCATTGTCCTGCTGGCAATTCCTATACTTCTTTT
GCCACTTATCACACTCCTGCAACAGATTGTTCTGATGGCAATTACAATCGTAATGCCAGTCTGAACTC
TTTTAAGGAGTATTTTAATTTACGTAACTGCACCTTTATGTACACTTATAACATTACCGAAGATGAGA
TTTTAGAGTGGTTTGGCATTACACAAACTGCTCAAGGTGTTCACCTCTTCTCATCTCGGTATGTTGAT
TTGTACGGCGGCAATATGTTTCAATTTGCCACCTTGCCTGTTTATGATACTATTAAGTATTATTCTAT
CATTCCTCACAGTATTCGTTCTATCCAAAGTGATAGAAAAGCTTGGGCTGCCTTCTACGTATATAAAC
TTCAACCGTTAACTTTCCTGTTGGATTTTTCTGTTGATGGTTATATACGCAGAGCTATAGACTGTGGT
TTTAATGATTTGTCACAACTCCACTGCTCATATGAATCCTTCGATGTTGAATCTGGAGTTTATTCAGT
TTCGTCTTTCGAAGCAAAACCTTCTGGCTCAGTIGTGGAACAGGCTGAAGGTGTTGAATGTGATTTTT
CACCTCTTCTGTCTGGCACACCTCCTCAGGTTTATAATTTCAAGCGTTTGGTTTTTACCAATTGCAAT
TATAATCTTACCAAATTGCTTTCACTTTTTTCTGTGAATGATTTTACTTGTAGTCAAATATCTCCAGC
AGCAATTGCTAGCAACTGTTATTCTTCACTGATTTTGGATTACTTTTCATACCCACTTAGTATGAAAT
CCGATCTCAGTGTTAGTTCTGCTGGTCCAATATCCCAGTTTAATTATAAACAGTCCTTTTCTAATCCC
ACATGTTTGATTTTAGCGACTGTTCCTCATAACCTTACTACTATTACTAAGCCTCTTAAGTACAGCTA
TATTAACAAGTGCTCTCGTCTTCTTTCTGATGATCGTACTGAAGTACCTCAGTTAGTGAACGCTAATC
AATACTCACCCTGTGTATCCATTGTCCCATCCACTGTGTGGGAAGACGGTGATTATTATAGGAAACAA
CTATCTCCACTTGAAGGTGGTGGCTGGCTTGTTGCTAGTGGCTCAACTGTTGCCATGACTGAGCAATT
ACAGATGGGCTTTGGTATTACAGTTCAATATGGTACAGACACCAATAGTGTTTGCCCCAAGCTTGAAT
TTGCTAATGACACAAAAATTGCCTCTCAATTAGGCAATTGCGTGGAATATTCCCTCTATGGTGTTTCG
GGCCGTGGTGTTTTTCAGAATTGCACAGCTGTAGGTGTTCGACAGCAGCGCTTTGTTTATGATGCGTA
CCAGAATTTAGTTGGCTATTATTCTGATGATGGCAACTACTACTGTTTGCGTGCTTGTGTTAGTGTTC
CTGTTTCTGTCATCTATGATAAAGAAACTAAAACCCACGCTACTCTATTTGGTAGTGTTGCATGTGAA
CACATTTCTTCTACCATGTCTCAATACTCCCGTTCTACGCGATCAATGCTTAAACGGCGAGATTCTAC
ATATGGCCCCCTTCAGACACCTGTTGGTTGTGTCCTAGGACTTGTTAATTCCTCTTTGTTCGTAGAGG
ACTGCAAGTTGCCTCTTGGTCAATCTCTCTGTGCTCTTCCTGACACACCTAGTACTCTCACACCTCGC
AGTGTGCGCTCTGTTCCAGGTGAAATGCGCTTGGCATCCATTGCTTTTAATCATCCTATTCAGGTTGA
TCAACTTAATAGTAGTTATTTTAAATTAAGTATACCCACTAATTTTTCCTTTGGTGTGACTCAGGAGT
ACATTCAGACAACCATTCAGAAAGTTACTGTTGATTGTAAACAGTACGTTTGCAATGGTTTCCAGAAG
TGTGAGCAATTACTGCGCGAGTATGGCCAGTTTTGTTCCAAAATAAACCAGGCTCTCCATGGTGCCAA
TTTACGCCAGGATGATTCTGTACGTAATTTGTTTGCGAGCGTGAAAAGCTCTCAATCATCTCCTATCA
TACCAGGTTTTGGAGGTGACTTTAATTTGACACTTCTAGAACCTGTTTCTATATCTACTGGCAGTCGT
AGTGCACGTAGTGCTATTGAGGATTTGCTATTTGACAAAGTCACTATAGCTGATCCTGGTTATATGCA
AGGTTACGATGATTGCATGCAGCAAGGTCCAGCATCAGCTCGTGATCTTATTTGTGCTCAATATGTGG
CTGGTTACAAAGTATTACCTCCTCTTATGGATGTTAATATGGAAGCCGCGTATACTTCATCTTTGCTT
GGCAGCATAGCAGGTGTTGGCTGGACTGCTGGCTTATCCTCCTTTGCTGCTATTCCATTTGCACAGAG
TATCTTTTATAGGTTAAACGGTGTTGGCATTACTCAACAGGTTCTTTCAGAGAACCAAAAGCTTATTG
CCAATAAGTTTAATCAGGCTCTGGGAGCTATGCAAACAGGCTTCACTACAACTAATGAAGCTTTTCAG
AAGGTTCAGGATGCTGTGAACAACAATGCACAGGCTCTATCCAAATTAGCTAGCGAGCTATCTAATAC
TTTTGGTGCTATTTCCGCCTCTATTGGAGACATCATACAACGTCTTGATGTTCTCGAACAGGACGCCC
AAATAGACAGACTTATTAATGGCCGTTTGACAACACTAAATGCTTTTGTTGCACAGCAGCTTGTTCGT
TCCGAATCAGCTGCTCTTTCCGCTCAATTGGCTAAAGATAAAGTCAATGAGTGTGTCAAGGCACAATC
CAAGCGTTCTGGATTTTGCGGTCAAGGCACACATATAGTGTCCTTTGTTGTAAATGCCCCTAATGGCC
TTTACTTCATGCATGTTGGTTATTACCCTAGCAACCACATTGAGGTTGTTTCTGCTTATGGTCTTTGC
GATGCAGCTAACCCTACTAATTGTATAGCCCCTGTTAATGGCTACTTTATTAAAACTAATAACACTAG
GATTGTTGATGAGTGGTCATATACTGGCTCGTCCTTCTATGCACCTGAGCCCATTACCTCCCTTAATA
CTAAGTATGTTGCACCACAGGTGACATACCAAAACATTTCTACTAACCTCCCTCCTCCTCTTCTCGGC
AATTCCACCGGGATTGACTTCCAAGATGAGTTGGATGAGTTTTTCAAAAATGTTAGCACCAGTATACC
TAATTTTGGTTCCCTAACACAGATTAATACTACATTACTCGATCTTACCTACGAGATGTTGTCTCTTC
AACAAGTTGTTAAAGCCCTTAATGAGTCTTACATAGACCTTAAAGAGCTTGGCAATTATACTTATTAC
AACAAATGGCCGTGGTACATTTGGCTTGGTTTCATTGCTGGGCTTGTTGCCTTAGCTCTATGCGTCTT
CTTCATACTGTGCTGCACTGGTTGTGGCACAAACTGTATGGGAAAACTTAAGTGTAATCGTTGTTGTG
ATAGATACGAGGAATACGACCTCGAGCCGCATAAGGTTCATGTTCACTAA
SEQ ID NO: 19 is a MERS-CoV Spike Glycoprotein, Nucleotide Sequence,
Codon Optimized For Expression In Human Cells
ATGATCCACAGCGTGTTCCTGCTGATGTTTCTGCTGACACCTACCGAGTCCTACGTGGATGTGGGCCC
AGACTCTGTGAAGAGCGCCTGCATCGAGGTGGACATCCAGCAGACATTCTTTGACAAGACCTGGCCCA
GACCCATCGACGTGAGCAAGGCAGACGGAATCATCTACCCACAGGGACGCACATATAGCAACATCACA
ATCACCTACCAGGGCCTGTTCCCTTATCAGGGCGACCACGGCGATATGTACGTGTATAGCGCCGGCCA
CGCAACCGGCACCACACCACAGAAGCTGTTTGTGGCCAATTATTCCCAGGACGTGAAGCAGTTCGCCA
ACGGATTTGTGGTGCGGATCGGGGCCGCCGCCAACAGCACAGGCACCGTGATCATCTCTCCCAGCACA
TCCGCCACCATCAGAAAGATCTACCCTGCCTTTATGCTGGGCAGCTCCGTGGGCAACTTCTCCGATGG
CAAGATGGGCAGGTTCTTTAATCACACACTGGTGCTGCTGCCAGACGGATGCGGCACCCTGCTGAGGG
CCTTCTACTGTATCCTGGAGCCCCGCTCTGGAAATCACTGCCCTGCCGGCAACTCCTACACCTCTTTT
GCCACATATCACACCCCTGCCACAGACTGTTCCGATGGCAATTATAACCGGAATGCCAGCCTGAACTC
CTTCAAGGAGTACTTTAATCTGAGAAACTGCACCTTCATGTACACATATAATATCACCGAGGATGAGA
TCCTGGAGTGGTTCGGCATCACACAGACCGCCCAGGGCGTGCACCTGTTTTCTAGCAGATACGTGGAT
CTGTATGGCGGCAACATGTTCCAGTTTGCCACACTGCCAGTGTATGACACCATCAAGTACTATAGCAT
CATCCCCCACTCTATCCGGAGCATCCAGTCCGACAGAAAGGCCTGGGCCGCCTTCTACGTGTATAAGC
TGCAGCCCCTGACCTTCCTGCTGGATTTTTCCGTGGACGGCTACATCCGGAGAGCCATCGATTGCGGC
TTTAACGACCTGTCTCAGCTCCACTGTTCTTATGAGAGCTTCGATGTGGAGTCTGGCGTGTACAGCGT
GTCCTCTTTTGAGGCCAAGCCATCTGGCAGCGTGGTGGAGCAGGCAGAGGGAGTGGAGTGCGACTTCT
CCCCACTGCTGTCTGGCACACCACCTCAGGTGTATAATTTCAAGAGGCTGGTGTTTACAAACTGTAAT
TACAACCTGACCAAGCTGCTGTCCCTGTTCTCTGTGAACGACTTTACCTGCAGCCAGATCTCCCCTGC
CGCCATCGCCTCCAATTGTTATAGCTCCCTGATCCTGGATTACTTCTCTTATCCCCTGTCTATGAAGA
GCGACCTGTCCGTGTCTAGCGCCGGCCCTATCAGCCAGTTTAATTACAAGCAGTCCTTCTCTAACCCC
ACATGCCTGATCCTGGCCACCGTGCCTCACAACCTGACCACAATCACAAAGCCACTGAAGTACTCCTA
TATCAATAAGTGCAGCAGGCTGCTGTCCGACGATCGCACCGAGGTGCCTCAGCTCGTGAACGCCAACC
AGTACTCTCCATGCGTGAGCATCGTGCCATCCACCGTGTGGGAGGACGGCGATTACTATAGAAAGCAG
CTCAGCCCACTGGAGGGAGGAGGATGGCTGGTGGCCAGCGGCTCCACAGTGGCCATGACCGAGCAGCT
CCAGATGGGCTTCGGCATCACAGTGCAGTACGGCACAGATACCAATAGCGTGTGCCCCAAGCTGGAGT
TTGCCAACGACACCAAGATCGCCTCCCAGCTCGGCAATTGCGTGGAGTACTCCCTGTATGGCGTGTCT
GGCAGAGGCGTGTTCCAGAACTGTACAGCCGTGGGCGTGCGGCAGCAGCGGTTCGTGTACGATGCCTA
TCAGAACCTGGTGGGCTACTATAGCGACGATGGCAATTACTATTGCCTGAGGGCATGCGTGAGCGTGC
CCGTGAGCGTGATCTACGACAAGGAGACAAAGACCCACGCCACCCTGTTCGGCTCCGTGGCCTGCGAG
CACATCTCCTCTACAATGTCTCAGTATTCTAGGAGCACCCGCTCTATGCTGAAGAGGCGCGACAGCAC
ATACGGACCACTGCAGACCCCTGTGGGATGCGTGCTGGGCCTGGTGAACAGCAGCCTGTTTGTGGAGG
ATTGCAAGCTGCCACTGGGCCAGTCTCTGTGCGCACTGCCAGACACCCCCAGCACACTGACCCCACGG
TCTGTGAGAAGCGTGCCCGGAGAGATGAGACTGGCCAGCATCGCCTTCAATCACCCTATCCAGGTGGA
TCAGCTCAACAGCAGCTACTTTAAGCTGAGCATCCCAACAAACTTCTCCTTTGGCGTGACCCAGGAGT
ATATCCAGACCACAATCCAGAAGGTGACCGTGGACTGCAAGCAGTACGTGTGCAATGGCTTCCAGAAG
TGCGAGCAGCTCCTGAGGGAGTATGGCCAGTTTTGTTCCAAGATCAATCAGGCCCTGCACGGAGCCAA
CCTGAGGCAGGACGATTCCGTGAGAAACCTGTTCGCCTCTGTGAAGTCCTCTCAGAGCTCCCCTATCA
TCCCAGGCTTCGGCGGCGACTTCAACCTGACCCTGCTGGAGCCCGTGTCCATCTCTACCGGCAGCAGG
TCCGCCCGCAGCGCCATCGAGGATCTGCTGTTTGACAAGGTGACCATCGCCGACCCAGGCTACATGCA
GGGCTATGACGATTGCATGCAGCAGGGACCAGCCTCCGCCCGCGATCTGATCTGTGCCCAGTACGTGG
CCGGCTATAAGGTGCTGCCACCCCTGATGGACGTGAACATGGAGGCCGCCTATACATCTAGCCTGCTG
GGCAGCATCGCAGGAGTGGGATGGACCGCCGGCCTGTCCTCTTTCGCCGCAATCCCTTTTGCCCAGTC
TATCTTCTACCGGCTGAACGGCGTGGGCATCACACAGCAGGTGCTGAGCGAGAATCAGAAGCTGATCG
CCAATAAGTTCAACCAGGCCCTGGGGGCCATGCAGACCGGCTTTACCACAACCAACGAGGCCTTCCAG
AAGGTGCAGGATGCCGTGAACAATAACGCACAGGCCCTGTCCAAGCTGGCCTCCGAGCTGTCTAATAC
CTTCGGGGCCATCAGCGCCAGCATCGGCGACATCATCCAGCGCCTGGACGTGCTGGAGCAGGATGCCC
AGATCGACAGGCTGATCAATGGCCGCCTGACAACCCTGAACGCCTTTGTGGCACAGCAGCTCGTGCGG
AGCGAGTCTGCCGCCCTGAGCGCCCAGCTCGCCAAGGACAAGGTGAACGAGTGCGTGAAGGCCCAGAG
CAAGCGGTCCGGCTTTTGTGGCCAGGGCACCCACATCGTGTCCTTCGTGGTGAATGCCCCTAACGGCC
TGTACTTTATGCATGTGGGCTACTATCCAAGCAACCACATCGAGGTGGTGTCCGCCTATGGCCTGTGC
GATGCCGCCAATCCTACAAACTGTATCGCCCCAGTGAATGGCTACTTCATCAAGACCAATAACACACG
GATCGTGGACGAGTGGTCCTACACCGGCAGCTCCTTTTATGCCCCCGAGCCTATCACATCTCTGAACA
CCAAGTACGTGGCCCCACAGGTGACATATCAGAATATCAGCACCAACCTGCCTCCACCCCTGCTGGGC
AATTCCACCGGCATCGACTTCCAGGATGAGCTGGACGAGTTCTTTAAGAATGTGAGCACATCCATCCC
CAACTTTGGCAGCCTGACCCAGATCAACACAACCCTGCTGGATCTGACATACGAGATGCTGTCTCTGC
AGCAGGTGGTGAAGGCCCTGAATGAGAGCTACATCGACCTGAAGGAGCTGGGCAATTATACCTACTAT
AACAAGTGGCCTTGGTACATCTGGCTGGGCTTCATCGCAGGCCTGGTGGCCCTGGCCCTGTGCGTGTT
CTTTATCCTGTGCTGTACAGGCTGCGGCACCAATTGTATGGGCAAGCTGAAGTGTAACCGGTGCTGTG
ATAGATACGAGGAGTATGACCTGGAGCCACACAAGGTGCATGTGCACTGA
SEQ ID NO: 20 is a MERS-CoV Spike Glycoprotein mRNA Sequence
AUGAUCCACAGCGUGUUCCUGCUGAUGUUUCUGCUGACACCUACCGAGUCCUACGUGGAUGUGGGCCC
AGACUCUGUGAAGAGCGCCUGCAUCGAGGUGGACAUCCAGCAGACAUUCUUUGACAAGACCUGGCCCA
GACCCAUCGACGUGAGCAAGGCAGACGGAAUCAUCUACCCACAGGGACGCACAUAUAGCAACAUCACA
AUCACCUACCAGGGCCUGUUCCCUUAUCAGGGCGACCACGGCGAUAUGUACGUGUAUAGCGCCGGCCA
CGCAACCGGCACCACACCACAGAAGCUGUUUGUGGCCAAUUAUUCCCAGGACGUGAAGCAGUUCGCCA
ACGGAUUUGUGGUGCGGAUCGGGGCCGCCGCCAACAGCACAGGCACCGUGAUCAUCUCUCCCAGCACA
UCCGCCACCAUCAGAAAGAUCUACCCUGCCUUUAUGCUGGGCAGCUCCGUGGGCAACUUCUCCGAUGG
CAAGAUGGGCAGGUUCUUUAAUCACACACUGGUGCUGCUGCCAGACGGAUGCGGCACCCUGCUGAGGG
CCUUCUACUGUAUCCUGGAGCCCCGCUCUGGAAAUCACUGCCCUGCCGGCAACUCCUACACCUCUUUU
GCCACAUAUCACACCCCUGCCACAGACUGUUCCGAUGGCAAUUAUAACCGGAAUGCCAGCCUGAACUC
CUUCAAGGAGUACUUUAAUCUGAGAAACUGCACCUUCAUGUACACAUAUAAUAUCACCGAGGAUGAGA
UCCUGGAGUGGUUCGGCAUCACACAGACCGCCCAGGGCGUGCACCUGUUUUCUAGCAGAUACGUGGAU
CUGUAUGGCGGCAACAUGUUCCAGUUUGCCACACUGCCAGUGUAUGACACCAUCAAGUACUAUAGCAU
CAUCCCCCACUCUAUCCGGAGCAUCCAGUCCGACAGAAAGGCCUGGGCCGCCUUCUACGUGUAUAAGC
UGCAGCCCCUGACCUUCCUGCUGGAUUUUUCCGUGGACGGCUACAUCCGGAGAGCCAUCGAUUGCGGC
UUUAACGACCUGUCUCAGCUCCACUGUUCUUAUGAGAGCUUCGAUGUGGAGUCUGGCGUGUACAGCGU
GUCCUCUUUUGAGGCCAAGCCAUCUGGCAGCGUGGUGGAGCAGGCAGAGGGAGUGGAGUGCGACUUCU
CCCCACUGCUGUCUGGCACACCACCUCAGGUGUAUAAUUUCAAGAGGCUGGUGUUUACAAACUGUAAU
UACAACCUGACCAAGCUGCUGUCCCUGUUCUCUGUGAACGACUUUACCUGCAGCCAGAUCUCCCCUGC
CGCCAUCGCCUCCAAUUGUUAUAGCUCCCUGAUCCUGGAUUACUUCUCUUAUCCCCUGUCUAUGAAGA
GCGACCUGUCCGUGUCUAGCGCCGGCCCUAUCAGCCAGUUUAAUUACAAGCAGUCCUUCUCUAACCCC
ACAUGCCUGAUCCUGGCCACCGUGCCUCACAACCUGACCACAAUCACAAAGCCACUGAAGUACUCCUA
UAUCAAUAAGUGCAGCAGGCUGCUGUCCGACGAUCGCACCGAGGUGCCUCAGCUCGUGAACGCCAACC
AGUACUCUCCAUGCGUGAGCAUCGUGCCAUCCACCGUGUGGGAGGACGGCGAUUACUAUAGAAAGCAG
CUCAGCCCACUGGAGGGAGGAGGAUGGCUGGUGGCCAGCGGCUCCACAGUGGCCAUGACCGAGCAGCU
CCAGAUGGGCUUCGGCAUCACAGUGCAGUACGGCACAGAUACCAAUAGCGUGUGCCCCAAGCUGGAGU
UUGCCAACGACACCAAGAUCGCCUCCCAGCUCGGCAAUUGCGUGGAGUACUCCCUGUAUGGCGUGUCU
GGCAGAGGCGUGUUCCAGAACUGUACAGCCGUGGGCGUGCGGCAGCAGCGGUUCGUGUACGAUGCCUA
UCAGAACCUGGUGGGCUACUAUAGCGACGAUGGCAAUUACUAUUGCCUGAGGGCAUGCGUGAGCGUGC
CCGUGAGCGUGAUCUACGACAAGGAGACAAAGACCCACGCCACCCUGUUCGGCUCCGUGGCCUGCGAG
CACAUCUCCUCUACAAUGUCUCAGUAUUCUAGGAGCACCCGCUCUAUGCUGAAGAGGCGCGACAGCAC
AUACGGACCACUGCAGACCCCUGUGGGAUGCGUGCUGGGCCUGGUGAACAGCAGCCUGUUUGUGGAGG
AUUGCAAGCUGCCACUGGGCCAGUCUCUGUGCGCACUGCCAGACACCCCCAGCACACUGACCCCACGG
UCUGUGAGAAGCGUGCCCGGAGAGAUGAGACUGGCCAGCAUCGCCUUCAAUCACCCUAUCCAGGUGGA
UCAGCUCAACAGCAGCUACUUUAAGCUGAGCAUCCCAACAAACUUCUCCUUUGGCGUGACCCAGGAGU
AUAUCCAGACCACAAUCCAGAAGGUGACCGUGGACUGCAAGCAGUACGUGUGCAAUGGCUUCCAGAAG
UGCGAGCAGCUCCUGAGGGAGUAUGGCCAGUUUUGUUCCAAGAUCAAUCAGGCCCUGCACGGAGCCAA
CCUGAGGCAGGACGAUUCCGUGAGAAACCUGUUCGCCUCUGUGAAGUCCUCUCAGAGCUCCCCUAUCA
UCCCAGGCUUCGGCGGCGACUUCAACCUGACCCUGCUGGAGCCCGUGUCCAUCUCUACCGGCAGCAGG
UCCGCCCGCAGCGCCAUCGAGGAUCUGCUGUUUGACAAGGUGACCAUCGCCGACCCAGGCUACAUGCA
GGGCUAUGACGAUUGCAUGCAGCAGGGACCAGCCUCCGCCCGCGAUCUGAUCUGUGCCCAGUACGUGG
CCGGCUAUAAGGUGCUGCCACCCCUGAUGGACGUGAACAUGGAGGCCGCCUAUACAUCUAGCCUGCUG
GGCAGCAUCGCAGGAGUGGGAUGGACCGCCGGCCUGUCCUCUUUCGCCGCAAUCCCUUUUGCCCAGUC
UAUCUUCUACCGGCUGAACGGCGUGGGCAUCACACAGCAGGUGCUGAGCGAGAAUCAGAAGCUGAUCG
CCAAUAAGUUCAACCAGGCCCUGGGGGCCAUGCAGACCGGCUUUACCACAACCAACGAGGCCUUCCAG
AAGGUGCAGGAUGCCGUGAACAAUAACGCACAGGCCCUGUCCAAGCUGGCCUCCGAGCUGUCUAAUAC
CUUCGGGGCCAUCAGCGCCAGCAUCGGCGACAUCAUCCAGCGCCUGGACGUGCUGGAGCAGGAUGCCC
AGAUCGACAGGCUGAUCAAUGGCCGCCUGACAACCCUGAACGCCUUUGUGGCACAGCAGCUCGUGCGG
AGCGAGUCUGCCGCCCUGAGCGCCCAGCUCGCCAAGGACAAGGUGAACGAGUGCGUGAAGGCCCAGAG
CAAGCGGUCCGGCUUUUGUGGCCAGGGCACCCACAUCGUGUCCUUCGUGGUGAAUGCCCCUAACGGCC
UGUACUUUAUGCAUGUGGGCUACUAUCCAAGCAACCACAUCGAGGUGGUGUCCGCCUAUGGCCUGUGC
GAUGCCGCCAAUCCUACAAACUGUAUCGCCCCAGUGAAUGGCUACUUCAUCAAGACCAAUAACACACG
GAUCGUGGACGAGUGGUCCUACACCGGCAGCUCCUUUUAUGCCCCCGAGCCUAUCACAUCUCUGAACA
CCAAGUACGUGGCCCCACAGGUGACAUAUCAGAAUAUCAGCACCAACCUGCCUCCACCCCUGCUGGGC
AAUUCCACCGGCAUCGACUUCCAGGAUGAGCUGGACGAGUUCUUUAAGAAUGUGAGCACAUCCAUCCC
CAACUUUGGCAGCCUGACCCAGAUCAACACAACCCUGCUGGAUCUGACAUACGAGAUGCUGUCUCUGC
AGCAGGUGGUGAAGGCCCUGAAUGAGAGCUACAUCGACCUGAAGGAGCUGGGCAAUUAUACCUACUAU
AACAAGUGGCCUUGGUACAUCUGGCUGGGCUUCAUCGCAGGCCUGGUGGCCCUGGCCCUGUGCGUGUU
CUUUAUCCUGUGCUGUACAGGCUGCGGCACCAAUUGUAUGGGCAAGCUGAAGUGUAACCGGUGCUGUG
AUAGAUACGAGGAGUAUGACCUGGAGCCACACAAGGUGCAUGUGCACUGA
SEQ ID NO: 21 is an HCMV gB-G Amino Acid Sequence
MESRIWCLVVCVNLCIVCLGAAVSSSSTRGTSATHSHHSSHTTSAAHSRSGSVSQRVTSSQTVSHGVN
ETIYNTTLKYGDVVGVNTTKYPYRVCSMAQGTDLIRFERNIVCTSMKPINEDLDEGIMVVYKRNIVAH
TFKVRVYQKVLTFRRSYAYIHTTYLLGSNTEYVAPPMWEIHHINSHSQCYSSYSRVIAGTVFVAYHRD
SYENKTMQLMPDDYSNTHSTRYVTVKDQWHSRGSTWLYRETCNLNCMVTITTARSKYPYHFFATSTGD
VVDISPFYNGTNRNASYFGENADKFFIFPNYTIVSDFGRPNSALETHRLVAFLERADSVISWDIQDEK
NVTCQLTFWEASERTIRSEAEDSYHESSAKMTATFLSKKQEVNMSDSALDCVRDEAINKLQQIENTSY
NQTYEKYGNVSVFETTGGLVVFWQGIKQKSLVELERLANRSSLNLTHNRTKRSTDGNNATHLSNMESV
HNLVYAQLQFTYDTLRGYINRALAQIAEAWCVDQRRTLEVFKELSKINPSAILSAIYNKPIAAREMGD
VLGLASCVTINQTSVKVLRDMNVKESPGRCYSRPVVIFNFANSSYVQYGQLGEDNEILLGNHRTEECQ
LPSLKIFIAGNSAYEYVDYLFKRMIDLSSISTVDSMIALDIDPLENTDERVLELYSQKELRSINVEDL
EEIMREFNSYKQRVKYVEDKVVDPLPPYLKGLDDLMSGLGAAGKAVGVAIGAVGGAVASVVEGVATEL
KNPFFFIIGLIIGLFLVLRVGIHLCIKLKHTKKRQIYTDIEMNRLGK* (TM and CTD underlined)
SEQ ID NO: 22 is an HCMV gB-G Nucleotide Sequence:
ATGGAATCCAGGATCTGGTGCCTGGTAGTCTGCGTTAACTTGTGTATCGTCTGTCTGGGTGCTGCGGT
TTCCTCATCTTCTACTCGTGGAACTTCTGCTACTCACAGTCACCATTCCTCTCATACGACGTCTGCTG
CTCATTCTCGATCCGGTTCAGTCTCTCAACGCGTAACTTCTTCCCAAACGGTCAGCCATGGTGTTAAC
GAGACCATCTACAACACTACCCTCAAGTACGGAGATGTGGTGGGGGTCAACACCACCAAGTACCCCTA
TCGCGTGTGTTCTATGGCACAGGGTACGGATCTTATTCGCTTTGAACGTAATATCGTCTGCACCTCGA
TGAAGCCCATCAATGAAGACCTGGACGAGGGCATCATGGTGGTCTACAAACGCAACATCGTCGCGCAC
ACCTTTAAGGTACGAGTCTACCAGAAGGTTTTGACGTTTCGTCGTAGCTACGCTTACATCCACACCAC
TTATCTGCTGGGCAGCAACACGGAATACGTGGCGCCTCCTATGTGGGAGATTCATCATATCAACAGTC
ACAGTCAGTGCTACAGTTCCTACAGCCGCGTTATAGCAGGCACGGTTTTCGTGGCTTATCATAGGGAC
AGCTATGAAAACAAAACCATGCAATTAATGCCCGACGATTATTCCAACACCCACAGTACCCGTTACGT
GACGGTCAAGGATCAATGGCACAGCCGCGGCAGCACCTGGCTCTATCGTGAGACCTGTAATCTGAATT
GTATGGTGACCATCACTACTGCGCGCTCCAAGTATCCCTATCATTTTTTCGCAACTTCCACGGGTGAT
GTGGTTGACATTTCTCCTTTCTACAACGGAACTAATCGCAATGCCAGCTATTTTGGAGAAAACGCCGA
CAAGTTTTTCATTTTTCCGAACTACACTATCGTCTCCGACTTTGGAAGACCGAATTCTGCGTTAGAGA
CCCACAGGTTGGTGGCTTTTCTTGAACGTGCGGACTCAGTGATCTCCTGGGATATACAGGACGAGAAG
AATGTTACTTGTCAACTCACTTTCTGGGAAGCCTCGGAACGCACCATTCGTTCCGAAGCCGAGGACTC
GTATCACTTTTCTTCTGCCAAAATGACCGCCACTTTCTTATCTAAGAAGCAAGAGGTGAACATGTCCG
ACTCTGCGCTGGACTGTGTACGTGATGAGGCCATAAATAAGTTACAGCAGATTTTCAATACTTCATAC
AATCAAACATATGAAAAATATGGAAACGTGTCCGTCTTTGAAACCACTGGTGGTTTGGTGGTGTTCTG
GCAAGGTATCAAGCAAAAATCTCTGGTGGAACTCGAACGTTTGGCCAACCGCTCCAGTCTGAATCTTA
CTCATAATAGAACCAAAAGAAGTACAGATGGCAACAATGCAACTCATTTATCCAACATGGAGTCGGTG
CACAATCTGGTCTACGCCCAGCTGCAGTTCACCTATGACACGTTGCGCGGTTACATCAACCGGGCGCT
GGCGCAAATCGCAGAAGCCTGGTGTGTGGATCAACGGCGCACCCTAGAGGTCTTCAAGGAACTTAGCA
AGATCAACCCGTCAGCTATTCTCTCGGCCATCTACAACAAACCGATTGCCGCGCGTTTCATGGGTGAT
GTCCTGGGTCTGGCCAGCTGCGTGACCATTAACCAAACCAGCGTCAAGGTGCTGCGTGATATGAATGT
GAAGGAATCGCCAGGACGCTGCTACTCACGACCAGTGGTCATCTTTAATTTCGCCAACAGCTCGTACG
TGCAGTACGGTCAACTGGGCGAGGATAACGAAATCCTGTTGGGCAACCACCGCACTGAGGAATGTCAG
CTTCCCAGCCTCAAGATCTTCATCGCCGGCAACTCGGCCTACGAGTACGTGGACTACCTCTTCAAACG
CATGATTGACCTCAGCAGCATCTCCACCGTCGACAGCATGATCGCCCTAGACATCGACCCGCTGGAAA
ACACCGACTTCAGGGTACTGGAACTTTACTCGCAGAAAGAATTGCGTTCCATCAACGTTTTTGATCTC
GAGGAGATCATGCGCGAGTTCAATTCGTATAAGCAGCGGGTAAAGTACGTGGAGGACAAGGTAGTCGA
CCCGCTGCCGCCCTACCTCAAGGGTCTGGACGACCTCATGAGCGGCCTGGGCGCCGCGGGAAAGGCCG
TTGGCGTAGCCATTGGGGCCGTGGGTGGCGCGGTGGCCTCCGTGGTCGAAGGCGTTGCCACCTTCCTC
AAAAACCCCTTTTTCTTTATCATAGGGTTAATCATTGGACTATTCTTGGTTCTCCGAGTTGGTATCCA
TCTTTGCATTAAATTAAAGCACACCAAGAAAAGACAGATTTATACAGACATAGAGATGAACCGACTTG
GAAAGTAA (TM and CTD underlined)
SEQ ID NO: 23 is an HCMV (gB-G) Codon Optimized Nucleotide Sequence
ATGGAGTCAAGGATTTGGTGTCTGGTCGTCTGCGTCAACCTGTGCATTGTCTGCCTGGGAGCCGCCGT
CTCATCATCATCTACCCGAGGCACATCCGCCACTCACTCTCACCATAGCTCCCATACCACATCCGCCG
CTCACTCAAGAAGCGGGTCCGTGTCTCAGAGGGTCACATCTAGTCAGACCGTGAGCCATGGAGTCAAC
GAGACAATCTACAATACTACCCTGAAGTATGGAGACGTGGTCGGCGTGAACACAACTAAATACCCCTA
TAGGGTCTGCTCTATGGCCCAGGGGACAGATCTGATCCGATTTGAACGGAACATCGTGTGCACTAGCA
TGAAGCCTATCAATGAGGACCTGGATGAAGGAATTATGGTGGTCTACAAACGAAATATCGTGGCCCAT
ACTTTTAAGGTGAGAGTCTATCAGAAAGTGCTGACCTTCCGGAGAAGCTACGCTTATATTCACACCAC
ATACCTGCTGGGGTCCAACACCGAGTATGTGGCACCCCCTATGTGGGAAATCCACCATATTAATAGTC
ATTCACAGTGCTACTCAAGCTACAGCAGAGTGATCGCTGGAACCGTGTTCGTCGCATACCACAGAGAC
AGTTATGAGAACAAGACAATGCAGCTCATGCCCGACGATTACAGTAATACCCATTCAACAAGATATGT
GACCGTCAAAGATCAGTGGCACTCTCGCGGCAGTACCTGGCTGTACCGAGAGACATGCAACCTGAATT
GTATGGTGACAATTACTACCGCCAGAAGCAAGTACCCTTATCACTTCTTTGCTACCTCAACAGGGGAC
GTGGTCGACATCAGCCCCTTCTACAACGGAACAAACCGGAATGCCTCCTATTTCGGCGAGAACGCTGA
CAAATTCTTTATCTTCCCCAACTACACTATCGTGAGCGATTTCGGCAGACCTAACAGTGCCCTGGAGA
CCCATCGGCTGGTGGCATTTCTGGAAAGAGCCGACAGCGTGATCTCCTGGGACATTCAGGATGAGAAG
AATGTGACCTGCCAGCTCACCTTCTGGGAGGCCAGCGAAAGAACCATCAGGTCCGAGGCAGAAGATTC
TTACCACTTTTCCTCTGCAAAAATGACTGCCACCTTCCTGTCCAAGAAACAGGAGGTGAACATGAGCG
ACTCCGCACTGGATTGCGTGCGGGACGAAGCCATCAATAAGCTGCAGCAGATCTTCAACACATCTTAC
AACCAGACTTACGAGAAGTACGGCAACGTGAGTGTCTTTGAAACAACTGGCGGGCTGGTGGTCTTCTG
GCAGGGGATCAAGCAGAAATCTCTGGTGGAGCTGGAACGGCTGGCCAATAGAAGTTCACTGAACCTGA
CTCATAATCGCACCAAGCGATCCACAGACGGAAACAATGCAACTCATCTGAGCAACATGGAGTCCGTG
CACAATCTGGTCTACGCCCAGCTCCAGTTCACTTACGACACCCTGCGAGGCTATATCAACCGGGCCCT
GGCTCAGATTGCAGAAGCCTGGTGCGTGGATCAGAGGCGCACCCTGGAGGTCTTTAAGGAACTGAGCA
AAATTAACCCATCTGCTATCCTGAGTGCAATCTACAATAAGCCCATCGCAGCCAGGTTCATGGGGGAC
GTGCTGGGACTGGCCTCCTGCGTCACTATCAACCAGACCTCTGTGAAGGTCCTGCGCGATATGAATGT
GAAAGAGAGTCCTGGCAGGTGTTATTCACGCCCAGTGGTCATCTTCAACTTCGCTAATAGCTCCTACG
TGCAGTATGGCCAGCTCGGGGAGGACAACGAAATCCTGCTGGGAAATCACAGGACCGAGGAATGTCAG
CTCCCAAGTCTGAAGATCTTTATTGCCGGCAACTCAGCTTACGAGTATGTGGATTACCTGTTCAAACG
CATGATCGACCTGTCTAGTATTTCAACAGTGGATAGCATGATCGCCCTGGACATTGATCCCCTGGAAA
ATACTGACTTCAGGGTGCTGGAGCTGTATAGCCAGAAGGAACTGCGCTCCATTAACGTGTTTGATCTG
GAGGAAATCATGAGGGAGTTCAATTCCTACAAGCAGCGCGTGAAATATGTCGAAGATAAGGTGGTCGA
CCCTCTGCCACCCTACCTGAAAGGCCTGGACGATCTGATGAGCGGGCTGGGAGCTGCAGGCAAGGCAG
TGGGAGTCGCCATCGGAGCTGTGGGAGGCGCTGTCGCATCCGTGGTCGAGGGAGTGGCTACCTTTCTG
AAGAACCCATTCTTTTTCATCATCGGCCTGATCATTGGGCTGTTCCTGGTGCTGAGAGTCGGCATCCA
CCTGTGCATTAAGCTGAAGCACACCAAGAAGAGGCAGATCTACACCGATATTGAAATGAACAGACTGG
GCAAGTGA (TM and CTD underlined)
SEQ ID NO: 24 is an HCMV (gB-G) mRNA Sequence
AUGGAGUCAAGGAUUUGGUGUCUGGUCGUCUGCGUCAACCUGUGCAUUGUCUGCCUGGGAGCCGCCGU
CUCAUCAUCAUCUACCCGAGGCACAUCCGCCACUCACUCUCACCAUAGCUCCCAUACCACAUCCGCCG
CUCACUCAAGAAGCGGGUCCGUGUCUCAGAGGGUCACAUCUAGUCAGACCGUGAGCCAUGGAGUCAAC
GAGACAAUCUACAAUACUACCCUGAAGUAUGGAGACGUGGUCGGCGUGAACACAACUAAAUACCCCUA
UAGGGUCUGCUCUAUGGCCCAGGGGACAGAUCUGAUCCGAUUUGAACGGAACAUCGUGUGCACUAGCA
UGAAGCCUAUCAAUGAGGACCUGGAUGAAGGAAUUAUGGUGGUCUACAAACGAAAUAUCGUGGCCCAU
ACUUUUAAGGUGAGAGUCUAUCAGAAAGUGCUGACCUUCCGGAGAAGCUACGCUUAUAUUCACACCAC
AUACCUGCUGGGGUCCAACACCGAGUAUGUGGCACCCCCUAUGUGGGAAAUCCACCAUAUUAAUAGUC
AUUCACAGUGCUACUCAAGCUACAGCAGAGUGAUCGCUGGAACCGUGUUCGUCGCAUACCACAGAGAC
AGUUAUGAGAACAAGACAAUGCAGCUCAUGCCCGACGAUUACAGUAAUACCCAUUCAACAAGAUAUGU
GACCGUCAAAGAUCAGUGGCACUCUCGCGGCAGUACCUGGCUGUACCGAGAGACAUGCAACCUGAAUU
GUAUGGUGACAAUUACUACCGCCAGAAGCAAGUACCCUUAUCACUUCUUUGCUACCUCAACAGGGGAC
GUGGUCGACAUCAGCCCCUUCUACAACGGAACAAACCGGAAUGCCUCCUAUUUCGGCGAGAACGCUGA
CAAAUUCUUUAUCUUCCCCAACUACACUAUCGUGAGCGAUUUCGGCAGACCUAACAGUGCCCUGGAGA
CCCAUCGGCUGGUGGCAUUUCUGGAAAGAGCCGACAGCGUGAUCUCCUGGGACAUUCAGGAUGAGAAG
AAUGUGACCUGCCAGCUCACCUUCUGGGAGGCCAGCGAAAGAACCAUCAGGUCCGAGGCAGAAGAUUC
UUACCACUUUUCCUCUGCAAAAAUGACUGCCACCUUCCUGUCCAAGAAACAGGAGGUGAACAUGAGCG
ACUCCGCACUGGAUUGCGUGCGGGACGAAGCCAUCAAUAAGCUGCAGCAGAUCUUCAACACAUCUUAC
AACCAGACUUACGAGAAGUACGGCAACGUGAGUGUCUUUGAAACAACUGGCGGGCUGGUGGUCUUCUG
GCAGGGGAUCAAGCAGAAAUCUCUGGUGGAGCUGGAACGGCUGGCCAAUAGAAGUUCACUGAACCUGA
CUCAUAAUCGCACCAAGCGAUCCACAGACGGAAACAAUGCAACUCAUCUGAGCAACAUGGAGUCCGUG
CACAAUCUGGUCUACGCCCAGCUCCAGUUCACUUACGACACCCUGCGAGGCUAUAUCAACCGGGCCCU
GGCUCAGAUUGCAGAAGCCUGGUGCGUGGAUCAGAGGCGCACCCUGGAGGUCUUUAAGGAACUGAGCA
AAAUUAACCCAUCUGCUAUCCUGAGUGCAAUCUACAAUAAGCCCAUCGCAGCCAGGUUCAUGGGGGAC
GUGCUGGGACUGGCCUCCUGCGUCACUAUCAACCAGACCUCUGUGAAGGUCCUGCGCGAUAUGAAUGU
GAAAGAGAGUCCUGGCAGGUGUUAUUCACGCCCAGUGGUCAUCUUCAACUUCGCUAAUAGCUCCUACG
UGCAGUAUGGCCAGCUCGGGGAGGACAACGAAAUCCUGCUGGGAAAUCACAGGACCGAGGAAUGUCAG
CUCCCAAGUCUGAAGAUCUUUAUUGCCGGCAACUCAGCUUACGAGUAUGUGGAUUACCUGUUCAAACG
CAUGAUCGACCUGUCUAGUAUUUCAACAGUGGAUAGCAUGAUCGCCCUGGACAUUGAUCCCCUGGAAA
AUACUGACUUCAGGGUGCUGGAGCUGUAUAGCCAGAAGGAACUGCGCUCCAUUAACGUGUUUGAUCUG
GAGGAAAUCAUGAGGGAGUUCAAUUCCUACAAGCAGCGCGUGAAAUAUGUCGAAGAUAAGGUGGUCGA
CCCUCUGCCACCCUACCUGAAAGGCCUGGACGAUCUGAUGAGCGGGCUGGGAGCUGCAGGCAAGGCAG
UGGGAGUCGCCAUCGGAGCUGUGGGAGGCGCUGUCGCAUCCGUGGUCGAGGGAGUGGCUACCUUUCUG
AAGAACCCAUUCUUUUUCAUCAUCGGCCUGAUCAUUGGGCUGUUCCUGGUGCUGAGAGUCGGCAUCCA
CCUGUGCAUUAAGCUGAAGCACACCAAGAAGAGGCAGAUCUACACCGAUAUUGAAAUGAACAGACUGG
GCAAGUGA
SEQ ID NO: 25 is an EGFP mRNA Sequence from TriLink BioTechnologies
AUGGUGAGCAAGGGCGAGGAGCUGUUCACCGGGGUGGUGCCCAUCCUGGUCGAGCUGGACGGCGACGU
AAACGGCCACAAGUUCAGCGUGUCCGGCGAGGGCGAGGGCGAUGCCACCUACGGCAAGCUGACCCUGA
AGUUCAUCUGCACCACCGGCAAGCUGCCCGUGCCCUGGCCCACCCUCGUGACCACCCUGACCUACGGC
GUGCAGUGCUUCAGCCGCUACCCCGACCACAUGAAGCAGCACGACUUCUUCAAGUCCGCCAUGCCCGA
AGGCUACGUCCAGGAGCGCACCAUCUUCUUCAAGGACGACGGCAACUACAAGACCCGCGCCGAGGUGA
AGUUCGAGGGCGACACCCUGGUGAACCGCAUCGAGCUGAAGGGCAUCGACUUCAAGGAGGACGGCAAC
AUCCUGGGGCACAAGCUGGAGUACAACUACAACAGCCACAACGUCUAUAUCAUGGCCGACAAGCAGAA
GAACGGCAUCAAGGUGAACUUCAAGAUCCGCCACAACAUCGAGGACGGCAGCGUGCAGCUCGCCGACC
ACUACCAGCAGAACACCCCCAUCGGCGACGGCCCCGUGCUGCUGCCCGACAACCACUACCUGAGCACC
CAGUCCGCCCUGAGCAAAGACCCCAACGAGAAGCGCGAUCACAUGGUCCUGCUGGAGUUCGUGACCGC
CGCCGGGAUCACUCUCGGCAUGGACGAGCUGUACAAGUAA
SEQ ID NO: 26 is a SARS CoV 2 Spike Glycoprotein, Amino Acid Sequences
(COVID-v4 FURIN CLEAVAGE Mod. & VSV-G TMCyt SWAP)
MFVFLVLLPLVSSQCVNLTTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVEKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLERKSNLKPFERDISTEIYQAG
STPCNGVEGENCYFPLQSYGFQPTNGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PGSASSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDEGGENFSQILPDPSKPSKRS
FIEDLLENKVTLADAGFIKQYGDCLGDIAARDLICAQKENGLTVLPPLLTDEMIAQYTSALLAGTITS
GWTFGAGAALQIPFAMQMAYRENGIGVTQNVLYENQKLIANQFNSAIGKIQDSLSSTASALGKLQDVV
NQNAQALNTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKFFFIIGLIIGLEL
VLRVGIHLCIKLKHTKKRQIYTDIEMNRLGK
SEQ ID NO: 27-SARS CoV 2 Spike Glycoprotein, Nucleotide Sequences
(COVID-v4 FURIN CLEAVAGE Mod. & VSV-G TMCyt SWAP)
ATGTTTGTTTTTCTTGTTTTATTGCCACTAGTCTCTAGTCAGTGTGTTAATCTTACAACCAGAACTCA
ATTACCCCCTGCATACACTAATTCTTTCACACGTGGTGTTTATTACCCTGACAAAGTTTTCAGATCCT
CAGTTTTACATTCAACTCAGGACTTGTTCTTACCTTTCTTTTCCAATGTTACTTGGTTCCATGCTATA
CATGTCTCTGGGACCAATGGTACTAAGAGGTTTGATAACCCTGTCCTACCATTTAATGATGGTGTTTA
TTTTGCTTCCACTGAGAAGTCTAACATAATAAGAGGCTGGATTTTTGGTACTACTTTAGATTCGAAGA
CCCAGTCCCTACTTATTGTTAATAACGCTACTAATGTTGTTATTAAAGTCTGTGAATTTCAATTTTGT
AATGATCCATTTTTGGGTGTTTATTACCACAAAAACAACAAAAGTTGGATGGAAAGTGAGTTCAGAGT
TTATTCTAGTGCGAATAATTGCACTTTTGAATATGTCTCTCAGCCTTTTCTTATGGACCTTGAAGGAA
AACAGGGTAATTTCAAAAATCTTAGGGAATTTGTGTTTAAGAATATTGATGGTTATTTTAAAATATAT
TCTAAGCACACGCCTATTAATTTAGTGCGTGATCTCCCTCAGGGTTTTTCGGCTTTAGAACCATTGGT
AGATTTGCCAATAGGTATTAACATCACTAGGTTTCAAACTTTACTTGCTTTACATAGAAGTTATTTGA
CTCCTGGTGATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGTGCACTTGACCC
TCTCTCAGAAACAAAGTGTACGTTGAAATCCTTCACTGTAGAAAAAGGAATCTATCAAACTTCTAACT
TTAGAGTCCAACCAACAGAATCTATTGTTAGATTTCCTAATATTACAAACTTGTGCCCTTTTGGTGAA
GTTTTTAACGCCACCAGATTTGCATCTGTTTATGCTTGGAACAGGAAGAGAATCAGCAACTGTGTTGC
TGATTATTCTGTCCTATATAATTCCGCATCATTTTCCACTTTTAAGTGTTATGGAGTGTCTCCTACTA
AATTAAATGATCTCTGCTTTACTAATGTCTATGCAGATTCATTTGTAATTAGAGGTGATGAAGTCAGA
CAAATCGCTCCAGGGCAAACTGGAAAGATTGCTGATTATAATTATAAATTACCAGATGATTTTACAGG
CTGCGTTATAGCTTGGAATTCTAACAATCTTGATTCTAAGGTTGGTGGTAATTATAATTACCTGTATA
GATTGTTTAGGAAGTCTAATCTCAAACCTTTTGAGAGAGATATTTCAACTGAAATCTATCAGGCCGGT
AGCACACCTTGTAATGGTGTTGAAGGTTTTAATTGTTACTTTCCTTTACAATCATATGGTTTCCAACC
CACTAATGGTGTTGGTTACCAACCATACAGAGTAGTAGTACTTTCTTTTGAACTTCTACATGCACCAG
CAACTGTTTGTGGACCTAAAAAGTCTACTAATTTGGTTAAAAACAAATGTGTCAATTTCAACTTCAAT
GGTTTAACAGGCACAGGTGTTCTTACTGAGTCTAACAAAAAGTTTCTGCCTTTCCAACAATTTGGCAG
AGACATTGCTGACACTACTGATGCTGTCCGTGATCCACAGACACTTGAGATTCTTGACATTACACCAT
GTTCTTTTGGTGGTGTCAGTGTTATAACACCAGGAACAAATACTTCTAACCAGGTTGCTGTTCTTTAT
CAGGATGTTAACTGCACAGAAGTCCCTGTTGCTATTCATGCAGATCAACTTACTCCTACTTGGCGTGT
TTATTCTACAGGTTCTAATGTTTTTCAAACACGTGCAGGCTGTTTAATAGGGGCTGAACATGTCAACA
ACTCATATGAGTGTGACATACCCATTGGTGCAGGTATATGCGCTAGTTATCAGACTCAGACTAATTCT
CCTGGTAGTGCAAGTAGTGTAGCTAGTCAATCCATCATTGCCTACACTATGTCACTTGGTGCAGAAAA
TTCAGTTGCTTACTCTAATAACTCTATTGCCATACCCACAAATTTTACTATTAGTGTTACCACAGAAA
TTCTACCAGTGTCTATGACCAAGACATCAGTAGATTGTACAATGTACATTTGTGGTGATTCAACTGAA
TGCAGCAATCTTTTGTTGCAATATGGCAGTTTTTGTACACAATTAAACCGTGCTTTAACTGGAATAGC
TGTTGAACAAGACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAGCAAGAGGTCA
TTTATTGAAGATCTACTTTTCAACAAAGTGACACTTGCAGATGCTGGCTTCATCAAACAATATGGTGA
TTGCCTTGGTGATATTGCTGCTAGAGACCTCATTTGTGCACAAAAGTTTAACGGCCTTACTGTTTTGC
CACCTTTGCTCACAGATGAAATGATTGCTCAATACACTTCTGCACTGTTAGCGGGTACAATCACTTCT
GGTTGGACCTTTGGTGCAGGTGCTGCATTACAAATACCATTTGCTATGCAAATGGCTTATAGGTTTAA
TGGTATTGGAGTTACACAGAATGTTCTCTATGAGAACCAAAAATTGATTGCCAACCAATTTAATAGTG
CTATTGGCAAAATTCAAGACTCACTTTCTTCCACAGCAAGTGCACTTGGAAAACTTCAAGATGTGGTC
AACCAAAATGCACAAGCTTTAAACACGCTTGTTAAACAACTTAGCTCCAATTTTGGTGCAATTTCAAG
TGTTTTAAATGATATCCTTTCACGTCTTGACCCTCCTGAGGCTGAAGTGCAAATTGATAGGTTGATCA
CAGGCAGACTTCAAAGTTTGCAGACATATGTGACTCAACAATTAATTAGAGCTGCAGAAATCAGAGCT
TCTGCTAATCTTGCTGCTACTAAAATGTCAGAGTGTGTACTTGGACAATCAAAAAGAGTTGATTTTTG
TGGAAAGGGCTATCATCTTATGTCCTTCCCTCAGTCAGCACCTCATGGTGTAGTCTTCTTGCATGTGA
CTTATGTCCCTGCACAAGAAAAGAACTTCACAACTGCTCCTGCCATTTGTCATGATGGAAAAGCACAC
TTTCCTCGTGAAGGTGTCTTTGTTTCAAATGGCACACACTGGTTTGTAACACAAAGGAATTTTTATGA
ACCACAAATCATTACTACAGACAACACATTTGTGTCTGGTAACTGTGATGTTGTAATAGGAATTGTCA
ACAACACAGTTTATGATCCTTTGCAACCTGAATTAGACTCATTCAAGGAGGAGTTAGATAAATATTTT
AAGAATCATACATCACCAGATGTTGATTTAGGTGACATCTCTGGCATTAATGCTTCAGTTGTAAACAT
TCAAAAAGAAATTGACCGCCTCAATGAGGTTGCCAAGAATTTAAATGAATCTCTCATCGATCTCCAAG
AACTTGGAAAGTATGAGCAGTATATAAAATTTTTCTTTATCATAGGGTTAATCATTGGACTATTCTTG
GTTCTCCGAGTTGGTATCCATCTTTGCATTAAATTAAAGCACACCAAGAAAAGACAGATTTATACAGA
CATAGAGATGAACCGACTTGGAAAGTAA
SEQ ID NO: 28-SARS CoV 2 Spike Glycoprotein, Codon Optimized Sequences
(COVID-v4 FURIN CLEAVAGE Mod. & VSV-G TMCyt SWAP)
ATGTTCGTGTTCCTGGTGCTGCTGCCTCTGGTGAGCTCCCAGTGCGTGAACCTGACCACAAGGACCCA
GCTCCCCCCTGCCTATACCAATTCCTTTACAAGGGGCGTGTACTATCCAGACAAGGTGTTCCGCTCTA
GCGTGCTGCACTCTACACAGGATCTGTTCCTGCCCTTCTTTAGCAACGTGACCTGGTTTCACGCCATC
CATGTGAGCGGCACCAATGGCACAAAGCGGTTTGACAATCCTGTGCTGCCATTCAACGATGGCGTGTA
CTTTGCCTCCACCGAGAAGTCTAACATCATCAGAGGCTGGATCTTCGGCACCACACTGGACAGCAAGA
CACAGTCCCTGCTGATCGTGAACAATGCCACCAACGTGGTCATCAAGGTGTGCGAGTTTCAGTTCTGT
AATGATCCTTTTCTGGGCGTGTACTATCACAAGAACAATAAGTCTTGGATGGAGAGCGAGTTCCGCGT
GTATTCCTCTGCCAACAATTGTACATTCGAGTACGTGTCCCAGCCATTTCTGATGGACCTGGAGGGCA
AGCAGGGCAACTTCAAGAACCTGCGGGAGTTCGTGTTCAAGAATATCGATGGCTATTTCAAGATCTAC
TCTAAGCACACCCCTATCAACCTGGTGCGCGACCTGCCACAGGGCTTTAGCGCCCTGGAGCCTCTGGT
GGATCTGCCAATCGGCATCAACATCACCAGGTTCCAGACACTGCTGGCCCTGCACCGCAGCTACCTGA
CACCTGGCGACAGCTCCTCTGGATGGACCGCCGGGGCCGCCGCCTACTATGTGGGCTATCTGCAGCCA
CGGACCTTTCTGCTGAAGTACAACGAGAATGGCACCATCACAGACGCAGTGGATTGCGCCCTGGACCC
CCTGAGCGAGACCAAGTGTACACTGAAGTCCTTCACCGTGGAGAAGGGCATCTATCAGACATCCAATT
TTCGGGTGCAGCCCACCGAGTCTATCGTGAGATTCCCAAATATCACAAACCTGTGCCCCTTCGGCGAG
GTGTTTAACGCCACCAGATTCGCCAGCGTGTACGCCTGGAATCGGAAGAGAATCTCTAACTGCGTGGC
CGACTATAGCGTGCTGTACAACTCTGCCAGCTTTTCCACCTTCAAGTGCTATGGCGTGTCCCCCACAA
AGCTGAATGACCTGTGCTTCACCAACGTGTACGCCGATTCTTTTGTGATCAGGGGCGACGAGGTGAGA
CAGATCGCACCAGGCCAGACAGGCAAGATCGCCGACTACAATTATAAGCTGCCCGACGATTTCACCGG
CTGCGTGATCGCCTGGAACTCTAACAATCTGGATAGCAAAGTGGGCGGCAACTACAATTATCTGTACA
GGCTGTTCCGCAAGAGCAATCTGAAGCCTTTTGAGCGGGACATCTCTACAGAGATCTACCAGGCCGGC
AGCACCCCATGCAATGGCGTGGAGGGCTTCAACTGTTATTTTCCCCTGCAGTCCTACGGCTTTCAGCC
TACCAACGGCGTGGGCTATCAGCCATACAGAGTGGTGGTGCTGAGCTTCGAGCTGCTGCACGCACCAG
CAACAGTGTGCGGACCTAAGAAGTCCACCAATCTGGTGAAGAACAAGTGCGTGAACTTCAACTTCAAC
GGCCTGACCGGCACAGGCGTGCTGACCGAGTCCAATAAGAAGTTTCTGCCCTTCCAGCAGTTTGGCCG
GGACATCGCCGATACCACAGACGCCGTGAGAGACCCCCAGACCCTGGAGATCCTGGACATCACACCTT
GCTCCTTCGGCGGCGTGTCTGTGATCACACCTGGCACCAATACAAGCAACCAGGTGGCCGTGCTGTAT
CAGGACGTGAATTGTACCGAGGTGCCAGTGGCCATCCACGCCGATCAGCTCACCCCCACATGGCGGGT
GTACTCCACCGGCTCTAACGTGTTCCAGACAAGAGCCGGCTGCCTGATCGGAGCCGAGCATGTGAACA
ATTCCTATGAGTGCGACATCCCCATCGGAGCCGGCATCTGTGCCTCTTACCAGACCCAGACAAACAGC
CCTGGCTCCGCCAGCTCCGTGGCCTCTCAGAGCATCATCGCCTATACCATGAGCCTGGGGGCCGAGAA
CAGCGTGGCCTACTCTAACAATAGCATCGCCATCCCCACCAACTTTACAATCTCTGTGACCACAGAGA
TCCTGCCTGTGAGCATGACCAAGACATCCGTGGACTGCACAATGTATATCTGTGGCGATTCCACCGAG
TGCTCTAACCTGCTGCTGCAGTACGGCAGCTTCTGTACCCAGCTCAACCGGGCCCTGACAGGAATCGC
AGTGGAGCAGGACAAGAACACACAGGAGGTGTTTGCCCAGGTGAAGCAGATCTACAAGACCCCACCCA
TCAAGGACTTCGGCGGCTTTAATTTCTCCCAGATCCTGCCAGATCCCTCCAAGCCATCTAAGCGGAGC
TTCATCGAGGACCTGCTGTTTAACAAGGTGACCCTGGCCGATGCCGGCTTTATCAAGCAGTATGGCGA
TTGCCTGGGCGACATCGCCGCCAGAGACCTGATCTGTGCCCAGAAGTTCAATGGCCTGACCGTGCTGC
CTCCACTGCTGACAGATGAGATGATCGCACAGTACACAAGCGCCCTGCTGGCCGGCACCATCACATCC
GGATGGACCTTCGGGGCCGGGGCCGCCCTGCAGATCCCCTTCGCCATGCAGATGGCCTATAGGTTTAA
CGGCATCGGCGTGACCCAGAATGTGCTGTACGAGAACCAGAAGCTGATCGCCAATCAGTTCAACTCCG
CCATCGGCAAGATCCAGGACAGCCTGTCTAGCACAGCCTCCGCCCTGGGCAAGCTGCAGGATGTGGTG
AATCAGAACGCCCAGGCCCTGAATACCCTGGTGAAGCAGCTCAGCAGCAACTTCGGGGCCATCAGCAG
CGTGCTGAACGACATCCTGAGCCGGCTGGACCCCCCTGAGGCAGAGGTGCAGATCGACAGGCTGATCA
CAGGCCGCCTGCAGTCTCTGCAGACCTATGTGACACAGCAGCTCATCAGGGCCGCCGAGATCAGAGCC
AGCGCCAATCTGGCCGCCACCAAGATGTCCGAGTGCGTGCTGGGCCAGTCTAAGCGCGTGGACTTCTG
TGGCAAGGGCTATCACCTGATGAGCTTTCCACAGTCCGCCCCCCACGGAGTGGTGTTCCTGCATGTGA
CCTACGTGCCTGCCCAGGAGAAGAACTTTACCACAGCCCCAGCCATCTGCCACGATGGCAAGGCACAC
TTCCCCAGGGAGGGCGTGTTCGTGAGCAACGGCACCCACTGGTTCGTGACACAGCGCAACTTCTACGA
GCCTCAGATCATCACCACAGACAATACATTCGTGTCTGGCAACTGTGACGTGGTCATCGGCATCGTGA
ACAATACCGTGTATGATCCCCTGCAGCCTGAGCTGGACAGCTTCAAGGAGGAGCTGGATAAGTACTTT
AAGAATCACACCTCCCCCGACGTGGATCTGGGCGACATCTCTGGCATCAATGCCAGCGTGGTGAACAT
CCAGAAGGAGATCGACAGGCTGAACGAGGTGGCCAAGAATCTGAACGAGAGCCTGATCGATCTGCAGG
AGCTGGGCAAGTATGAGCAGTACATCAAGTTCTTTTTCATCATCGGCCTGATCATCGGCCTGTTCCTG
GTGCTGCGCGTGGGCATCCACCTGTGCATCAAGCTGAAGCACACCAAGAAGAGGCAGATCTACACAGA
CATCGAGATGAACCGCCTGGGCAAGTGA
SEQ ID NO: 29-SARS CoV 2 Spike Glycoprotein, mRNA Sequences (COVID-
v4 FURIN CLEAVAGE Mod. & VSV-G TMCyt SWAP)
AUGUUCGUGUUCCUGGUGCUGCUGCCUCUGGUGAGCUCCCAGUGCGUGAACCUGACCACAAGGACCCA
GCUCCCCCCUGCCUAUACCAAUUCCUUUACAAGGGGCGUGUACUAUCCAGACAAGGUGUUCCGCUCUA
GCGUGCUGCACUCUACACAGGAUCUGUUCCUGCCCUUCUUUAGCAACGUGACCUGGUUUCACGCCAUC
CAUGUGAGCGGCACCAAUGGCACAAAGCGGUUUGACAAUCCUGUGCUGCCAUUCAACGAUGGCGUGUA
CUUUGCCUCCACCGAGAAGUCUAACAUCAUCAGAGGCUGGAUCUUCGGCACCACACUGGACAGCAAGA
CACAGUCCCUGCUGAUCGUGAACAAUGCCACCAACGUGGUCAUCAAGGUGUGCGAGUUUCAGUUCUGU
AAUGAUCCUUUUCUGGGCGUGUACUAUCACAAGAACAAUAAGUCUUGGAUGGAGAGCGAGUUCCGCGU
GUAUUCCUCUGCCAACAAUUGUACAUUCGAGUACGUGUCCCAGCCAUUUCUGAUGGACCUGGAGGGCA
AGCAGGGCAACUUCAAGAACCUGCGGGAGUUCGUGUUCAAGAAUAUCGAUGGCUAUUUCAAGAUCUAC
UCUAAGCACACCCCUAUCAACCUGGUGCGCGACCUGCCACAGGGCUUUAGCGCCCUGGAGCCUCUGGU
GGAUCUGCCAAUCGGCAUCAACAUCACCAGGUUCCAGACACUGCUGGCCCUGCACCGCAGCUACCUGA
CACCUGGCGACAGCUCCUCUGGAUGGACCGCCGGGGCCGCCGCCUACUAUGUGGGCUAUCUGCAGCCA
CGGACCUUUCUGCUGAAGUACAACGAGAAUGGCACCAUCACAGACGCAGUGGAUUGCGCCCUGGACCC
CCUGAGCGAGACCAAGUGUACACUGAAGUCCUUCACCGUGGAGAAGGGCAUCUAUCAGACAUCCAAUU
UUCGGGUGCAGCCCACCGAGUCUAUCGUGAGAUUCCCAAAUAUCACAAACCUGUGCCCCUUCGGCGAG
GUGUUUAACGCCACCAGAUUCGCCAGCGUGUACGCCUGGAAUCGGAAGAGAAUCUCUAACUGCGUGGC
CGACUAUAGCGUGCUGUACAACUCUGCCAGCUUUUCCACCUUCAAGUGCUAUGGCGUGUCCCCCACAA
AGCUGAAUGACCUGUGCUUCACCAACGUGUACGCCGAUUCUUUUGUGAUCAGGGGCGACGAGGUGAGA
CAGAUCGCACCAGGCCAGACAGGCAAGAUCGCCGACUACAAUUAUAAGCUGCCCGACGAUUUCACCGG
CUGCGUGAUCGCCUGGAACUCUAACAAUCUGGAUAGCAAAGUGGGCGGCAACUACAAUUAUCUGUACA
GGCUGUUCCGCAAGAGCAAUCUGAAGCCUUUUGAGCGGGACAUCUCUACAGAGAUCUACCAGGCCGGC
AGCACCCCAUGCAAUGGCGUGGAGGGCUUCAACUGUUAUUUUCCCCUGCAGUCCUACGGCUUUCAGCC
UACCAACGGCGUGGGCUAUCAGCCAUACAGAGUGGUGGUGCUGAGCUUCGAGCUGCUGCACGCACCAG
CAACAGUGUGCGGACCUAAGAAGUCCACCAAUCUGGUGAAGAACAAGUGCGUGAACUUCAACUUCAAC
GGCCUGACCGGCACAGGCGUGCUGACCGAGUCCAAUAAGAAGUUUCUGCCCUUCCAGCAGUUUGGCCG
GGACAUCGCCGAUACCACAGACGCCGUGAGAGACCCCCAGACCCUGGAGAUCCUGGACAUCACACCUU
GCUCCUUCGGCGGCGUGUCUGUGAUCACACCUGGCACCAAUACAAGCAACCAGGUGGCCGUGCUGUAU
CAGGACGUGAAUUGUACCGAGGUGCCAGUGGCCAUCCACGCCGAUCAGCUCACCCCCACAUGGCGGGU
GUACUCCACCGGCUCUAACGUGUUCCAGACAAGAGCCGGCUGCCUGAUCGGAGCCGAGCAUGUGAACA
AUUCCUAUGAGUGCGACAUCCCCAUCGGAGCCGGCAUCUGUGCCUCUUACCAGACCCAGACAAACAGC
CCUGGCUCCGCCAGCUCCGUGGCCUCUCAGAGCAUCAUCGCCUAUACCAUGAGCCUGGGGGCCGAGAA
CAGCGUGGCCUACUCUAACAAUAGCAUCGCCAUCCCCACCAACUUUACAAUCUCUGUGACCACAGAGA
UCCUGCCUGUGAGCAUGACCAAGACAUCCGUGGACUGCACAAUGUAUAUCUGUGGCGAUUCCACCGAG
UGCUCUAACCUGCUGCUGCAGUACGGCAGCUUCUGUACCCAGCUCAACCGGGCCCUGACAGGAAUCGC
AGUGGAGCAGGACAAGAACACACAGGAGGUGUUUGCCCAGGUGAAGCAGAUCUACAAGACCCCACCCA
UCAAGGACUUCGGCGGCUUUAAUUUCUCCCAGAUCCUGCCAGAUCCCUCCAAGCCAUCUAAGCGGAGC
UUCAUCGAGGACCUGCUGUUUAACAAGGUGACCCUGGCCGAUGCCGGCUUUAUCAAGCAGUAUGGCGA
UUGCCUGGGCGACAUCGCCGCCAGAGACCUGAUCUGUGCCCAGAAGUUCAAUGGCCUGACCGUGCUGC
CUCCACUGCUGACAGAUGAGAUGAUCGCACAGUACACAAGCGCCCUGCUGGCCGGCACCAUCACAUCC
GGAUGGACCUUCGGGGCCGGGGCCGCCCUGCAGAUCCCCUUCGCCAUGCAGAUGGCCUAUAGGUUUAA
CGGCAUCGGCGUGACCCAGAAUGUGCUGUACGAGAACCAGAAGCUGAUCGCCAAUCAGUUCAACUCCG
CCAUCGGCAAGAUCCAGGACAGCCUGUCUAGCACAGCCUCCGCCCUGGGCAAGCUGCAGGAUGUGGUG
AAUCAGAACGCCCAGGCCCUGAAUACCCUGGUGAAGCAGCUCAGCAGCAACUUCGGGGCCAUCAGCAG
CGUGCUGAACGACAUCCUGAGCCGGCUGGACCCCCCUGAGGCAGAGGUGCAGAUCGACAGGCUGAUCA
CAGGCCGCCUGCAGUCUCUGCAGACCUAUGUGACACAGCAGCUCAUCAGGGCCGCCGAGAUCAGAGCC
AGCGCCAAUCUGGCCGCCACCAAGAUGUCCGAGUGCGUGCUGGGCCAGUCUAAGCGCGUGGACUUCUG
UGGCAAGGGCUAUCACCUGAUGAGCUUUCCACAGUCCGCCCCCCACGGAGUGGUGUUCCUGCAUGUGA
CCUACGUGCCUGCCCAGGAGAAGAACUUUACCACAGCCCCAGCCAUCUGCCACGAUGGCAAGGCACAC
UUCCCCAGGGAGGGCGUGUUCGUGAGCAACGGCACCCACUGGUUCGUGACACAGCGCAACUUCUACGA
GCCUCAGAUCAUCACCACAGACAAUACAUUCGUGUCUGGCAACUGUGACGUGGUCAUCGGCAUCGUGA
ACAAUACCGUGUAUGAUCCCCUGCAGCCUGAGCUGGACAGCUUCAAGGAGGAGCUGGAUAAGUACUUU
AAGAAUCACACCUCCCCCGACGUGGAUCUGGGCGACAUCUCUGGCAUCAAUGCCAGCGUGGUGAACAU
CCAGAAGGAGAUCGACAGGCUGAACGAGGUGGCCAAGAAUCUGAACGAGAGCCUGAUCGAUCUGCAGG
AGCUGGGCAAGUAUGAGCAGUACAUCAAGUUCUUUUUCAUCAUCGGCCUGAUCAUCGGCCUGUUCCUG
GUGCUGCGCGUGGGCAUCCACCUGUGCAUCAAGCUGAAGCACACCAAGAAGAGGCAGAUCUACACAGA
CAUCGAGAUGAACCGCCUGGGCAAGUGA
SEQ ID NO: 30-SARS CoV-1 Spike Glycoprotein, Amino Acid Sequences
(Prefusion(2PP))
MFIFLLELTLTSGSDLDRCTTFDDVQAPNYTQHTSSMRGVYYPDEIFRSDTLYLTQDLFLPFYSNVTG
FHTINHTFGNPVIPFKDGIYFAATEKSNVVRGWVFGSTMNNKSQSVIIINNSTNVVIRACNFELCDNP
FFAVSKPMGTQTHTMIFDNAFNCTFEYISDAFSLDVSEKSGNFKHLREFVFKNKDGFLYVYKGYQPID
VVRDLPSGENTLKPIFKLPLGINITNFRAILTAFSPAQDIWGTSAAAYFVGYLKPTTFMLKYDENGTI
TDAVDCSQNPLAELKCSVKSFEIDKGIYQTSNFRVVPSGDVVRFPNITNLCPFGEVENATKEPSVYAW
ERKKISNCVADYSVLYNSTFFSTFKCYGVSATKLNDLCFSNVYADSFVVKGDDVRQIAPGQTGVIADY
NYKLPDDEMGCVLAWNTRNIDATSTGNYNYKYRYLRHGKLRPFERDISNVPFSPDGKPCTPPALNCYW
PLNDYGFYTTTGIGYQPYRVVVLSFELLNAPATVCGPKLSTDLIKNQCVNFNENGLTGTGVLTPSSKR
FQPFQQFGRDVSDFTDSVRDPKTSEILDISPCSFGGVSVITPGTNASSEVAVLYQDVNCTDVSTAIHA
DQLTPAWRIYSTGNNVFQTQAGCLIGAEHVDTSYECDIPIGAGICASYHTVSLLRSTSQKSIVAYTMS
LGADSSIAYSNNTIAIPTNFSISITTEVMPVSMAKTSVDCNMYICGDSTECANLLLQYGSFCTQLNRA
LSGIAAEQDRNTREVFAQVKQMYKTPTLKYFGGFNFSQILPDPLKPTKRSFIEDLLENKVTLADAGEM
KQYGECLGDINARDLICAQKENGLTVLPPLLTDDMIAAYTAALVSGTATAGWTFGAGAALQIPFAMQM
AYRFNGIGVTQNVLYENQKQIANQFNKAISQIQESLTTTSTALGKLQDVVNQNAQALNTLVKQLSSNF
GAISSVLNDILSRLDPPEAEVQIDRLITGRLQSLQTYVTQQLIRAAEIRASANLAATKMSECVLGQSK
RVDFCGKGYHLMSFPQAAPHGVVELHVTYVPSQERNFTTAPAICHEGKAYFPREGVEVENGTSWFITQ
RNFFSPQIITTDNTFVSGNCDVVIGIINNTVYDPLQPELDSFKEELDKYFKNHTSPDVDLGDISGINA
SVVNIQKEIDRLNEVAKNLNESLIDLQELGKYEQYIKWPWYVWLGFIAGLIAIVMVTILLCCMTSCCS
CLKGACSCGSCCKFDEDDSEPVLKGVKLHYT
SEQ ID NO: 31-SARS CoV-1 Spike Glycoprotein, Nucleotide Sequences
(Prefusion(2PP))
ATGTTCATCTTTCTGCTGTTCCTGACCCTGACAAGCGGCTCCGACCTGGATAGGTGCACCACATTTGA
CGATGTGCAGGCCCCCAACTACACACAGCACACCAGCTCCATGAGGGGCGTGTACTATCCTGATGAGA
TCTTCCGCTCTGACACACTGTACCTGACCCAGGACCTGTTCCTGCCTTTTTATAGCAATGTGACAGGC
TTCCACACCATCAATCACACATTTGGCAACCCCGTGATCCCTTTCAAGGATGGCATCTACTTTGCCGC
CACCGAGAAGTCTAACGTGGTGCGGGGATGGGTGTTCGGCAGCACAATGAACAATAAGTCTCAGAGCG
TGATCATCATCAACAATAGCACCAACGTGGTCATCAGAGCCTGCAATTTTGAGCTGTGCGACAACCCC
TTCTTTGCCGTGTCCAAGCCTATGGGCACCCAGACACACACCATGATCTTTGATAATGCCTTCAACTG
TACCTTTGAGTACATCAGCGATGCCTTTTCCCTGGACGTGTCTGAGAAGTCCGGCAACTTCAAGCACC
TGAGGGAGTTCGTGTTTAAGAATAAGGACGGCTTCCTGTACGTGTATAAGGGCTATCAGCCCATCGAT
GTGGTGCGCGACCTGCCTTCCGGCTTCAACACCCTGAAGCCAATCTTTAAGCTGCCCCTGGGCATCAA
TATCACCAACTTCAGGGCCATCCTGACAGCCTTTAGCCCAGCACAGGACATCTGGGGCACCAGCGCCG
CCGCCTACTTCGTGGGCTATCTGAAGCCCACCACCTTCATGCTGAAGTACGATGAGAACGGCACAATC
ACCGACGCCGTGGATTGCAGCCAGAATCCACTGGCCGAGCTGAAGTGTTCCGTGAAGTCTTTCGAGAT
CGACAAGGGCATCTATCAGACCTCCAACTTTAGGGTGGTGCCATCTGGCGATGTGGTGCGCTTCCCAA
ATATCACCAACCTGTGCCCCTTCGGCGAGGTGTTTAATGCCACAAAGTTCCCCAGCGTGTACGCCTGG
GAGCGCAAGAAGATCAGCAACTGCGTGGCCGACTACTCCGTGCTGTATAATAGCACCTTCTTCAGCAC
CTTCAAGTGCTACGGCGTGAGCGCCACCAAGCTGAATGACCTGTGCTTCTCTAACGTGTATGCCGATA
GCTTTGTGGTGAAGGGCGACGATGTGAGGCAGATCGCACCTGGACAGACCGGCGTGATCGCAGACTAC
AACTATAAGCTGCCAGACGATTTCATGGGCTGCGTGCTGGCCTGGAATACACGCAACATCGATGCCAC
ATCCACCGGCAACTACAATTATAAGTACCGGTATCTGAGACACGGCAAGCTGCGGCCCTTCGAGAGAG
ACATCTCCAATGTGCCATTTTCTCCAGATGGCAAGCCATGCACCCCACCTGCCCTGAATTGTTACTGG
CCTCTGAACGACTACGGCTTCTATACCACAACCGGCATCGGCTACCAGCCTTATAGGGTGGTGGTGCT
GTCCTTTGAGCTGCTGAACGCACCTGCAACCGTGTGCGGACCAAAGCTGTCTACAGATCTGATCAAGA
ATCAGTGCGTGAACTTCAACTTCAACGGCCTGACAGGCACCGGCGTGCTGACCCCTTCTAGCAAGCGG
TTCCAGCCATTTCAGCAGTTCGGCAGAGACGTGAGCGATTTCACCGACTCCGTGCGCGACCCAAAGAC
ATCCGAGATCCTGGACATCAGCCCCTGCTCCTTTGGCGGCGTGTCTGTGATCACACCTGGCACCAACG
CCTCCTCTGAGGTGGCCGTGCTGTACCAGGATGTGAATTGTACCGACGTGAGCACAGCAATCCACGCA
GACCAGCTCACCCCAGCATGGCGGATCTATTCCACCGGCAACAACGTGTTCCAGACACAGGCAGGATG
CCTGATCGGAGCCGAGCATGTGGATACAAGCTACGAGTGCGACATCCCCATCGGAGCCGGCATCTGTG
CCTCTTATCACACCGTGAGCCTGCTGAGATCCACATCTCAGAAGTCTATCGTGGCCTACACCATGAGC
CTGGGGGCCGATAGCTCCATCGCCTATTCCAACAATACCATCGCCATCCCAACAAACTTCAGCATCTC
CATCACAACCGAAGTGATGCCCGTGTCCATGGCCAAGACCTCTGTGGACTGCAACATGTACATCTGTG
GCGATAGCACAGAGTGCGCCAATCTGCTGCTGCAGTATGGCTCCTTTTGTACCCAGCTCAACCGGGCC
CTGTCTGGAATCGCCGCCGAGCAGGACAGGAATACACGCGAGGTGTTCGCCCAGGTGAAGCAGATGTA
CAAGACACCTACCCTGAAGTATTTTGGCGGCTTCAACTTTTCTCAGATCCTGCCTGATCCACTGAAGC
CAACCAAGCGGAGCTTCATCGAGGACCTGCTGTTTAATAAGGTGACACTGGCCGATGCCGGCTTCATG
AAGCAGTACGGCGAGTGCCTGGGCGACATCAACGCCAGAGACCTGATCTGTGCCCAGAAGTTTAATGG
CCTGACCGTGCTGCCACCCCTGCTGACAGACGATATGATCGCAGCATATACCGCCGCCCTGGTGTCCG
GCACAGCCACCGCCGGCTGGACCTTCGGGGCCGGGGCCGCCCTGCAGATCCCTTTCGCCATGCAGATG
GCCTACCGGTTTAACGGCATCGGCGTGACCCAGAATGTGCTGTATGAGAACCAGAAGCAGATCGCCAA
TCAGTTTAACAAGGCCATCAGCCAGATCCAGGAGTCCCTGACAACCACATCTACCGCCCTGGGCAAGC
TGCAGGACGTGGTGAATCAGAACGCCCAGGCCCTGAATACACTGGTGAAGCAGCTCAGCAGCAACTTC
GGGGCCATCAGCAGCGTGCTGAACGACATCCTGAGCCGGCTGGACCCGCCGGAGGCAGAGGTGCAGAT
CGATAGGCTGATCACCGGCAGACTGCAGTCTCTGCAGACATACGTGACCCAGCAGCTCATCAGGGCCG
CCGAGATCAGAGCCAGCGCCAACCTGGCCGCCACAAAGATGTCCGAGTGCGTGCTGGGCCAGTCTAAG
AGGGTGGACTTCTGTGGCAAGGGCTACCACCTGATGTCCTTTCCACAGGCCGCCCCTCACGGAGTGGT
GTTCCTGCATGTGACCTATGTGCCTTCTCAGGAGCGCAACTTTACCACAGCCCCAGCAATCTGCCACG
AGGGCAAGGCATACTTCCCCCGGGAGGGCGTGTTCGTGTTTAACGGCACCTCCTGGTTTATCACACAG
AGAAATTTCTTTTCCCCTCAGATCATCACCACAGACAATACCTTCGTGAGCGGCAACTGTGACGTGGT
CATCGGCATCATCAACAATACAGTGTACGATCCTCTGCAGCCAGAGCTGGACAGCTTCAAGGAGGAGC
TGGATAAGTACTTCAAGAACCACACCTCCCCCGACGTGGATCTGGGCGACATCAGCGGCATCAATGCC
TCCGTGGTGAACATCCAGAAGGAGATCGACAGACTGAATGAGGTGGCCAAGAATCTGAACGAGTCCCT
GATCGATCTGCAGGAGCTGGGCAAGTACGAGCAGTATATCAAGTGGCCATGGTACGTGTGGCTGGGCT
TCATCGCCGGCCTGATCGCCATCGTGATGGTGACCATCCTGCTGTGCTGTATGACATCTTGCTGTAGC
TGCCTGAAGGGAGCCTGCTCCTGTGGCTCTTGCTGTAAGTTTGACGAGGACGATAGCGAGCCCGTGCT
GAAGGGCGTGAAGCTGCACTATACCTGA
SEQ ID NO: 32-SARS CoV-1 Spike Glycoprotein, Codon Optimized Sequences
(Prefusion(2PP))
ATGTTCATCTTCCTGCTGTTCCTGACTCTGACCAGCGGATCTGATCTGGACCGGTGCACCACCTTTGA
CGACGTGCAGGCCCCTAACTACACCCAGCACACCTCCAGCATGAGAGGCGTGTACTACCCCGACGAGA
TTTTCAGATCAGACACCCTGTACCTCACCCAGGACCTGTTCCTGCCTTTCTACAGCAACGTGACCGGC
TTTCACACCATCAATCACACCTTCGGCAACCCTGTGATACCCTTCAAGGATGGGATCTACTTTGCCGC
CACAGAGAAAAGCAACGTGGTCCGGGGCTGGGTGTTTGGGTCTACAATGAACAACAAGAGCCAGAGCG
TGATCATCATCAACAATAGCACCAACGTGGTCATCAGAGCTTGTAATTTCGAGCTGTGCGACAACCCA
TTCTTCGCCGTGTCCAAGCCTATGGGCACCCAGACCCACACGATGATCTTCGATAATGCCTTCAATTG
CACCTTCGAGTACATCTCCGATGCTTTTTCTCTCGACGTCTCTGAGAAGTCCGGCAATTTCAAGCACC
TGAGAGAGTTCGTGTTCAAAAACAAAGACGGCTTCCTGTACGTGTACAAGGGCTACCAGCCAATCGAC
GTGGTGCGGGACCTGCCTAGCGGCTTTAACACACTGAAGCCCATCTTCAAGCTGCCTCTGGGCATCAA
CATCACAAACTTTAGAGCCATCCTGACCGCTTTTAGCCCTGCCCAGGACATCTGGGGCACCAGCGCCG
CTGCATACTTCGTGGGCTACCTGAAACCCACCACATTTATGCTGAAGTACGACGAGAACGGCACAATC
ACCGACGCCGTGGACTGCTCTCAGAACCCCCTGGCCGAGCTGAAATGTTCCGTGAAGAGCTTTGAAAT
CGATAAGGGCATCTACCAGACCAGCAACTTTCGGGTGGTGCCTTCTGGAGACGTGGTGCGCTTCCCTA
ACATTACAAACCTGTGTCCATTCGGCGAAGTGTTTAACGCCACCAAGTTCCCTTCTGTGTACGCCTGG
GAGAGAAAGAAAATCAGCAACTGCGTGGCCGATTACAGCGTGCTGTATAACAGTACATTTTTCAGCAC
ATTCAAGTGTTACGGCGTTAGCGCCACAAAGCTTAATGACTTGTGCTTCTCAAACGTGTACGCCGACA
GCTTCGTGGTCAAGGGCGACGACGTGCGGCAGATTGCCCCTGGTCAGACCGGCGTGATAGCCGATTAC
AACTACAAGCTGCCCGACGATTTCATGGGCTGCGTGCTGGCCTGGAACACCAGAAATATCGACGCCAC
AAGCACCGGCAATTACAACTACAAGTATAGATACCTGAGGCACGGCAAGCTGAGACCTTTCGAGAGAG
ACATCAGCAACGTGCCATTCAGCCCTGATGGCAAGCCTTGCACCCCACCTGCCCTCAACTGCTACTGG
CCCCTGAACGACTACGGCTTCTACACCACAACAGGCATTGGCTATCAGCCTTACAGAGTTGTGGTTCT
GAGCTTCGAACTGCTGAACGCTCCTGCTACCGTTTGTGGCCCTAAGCTGAGCACCGACCTGATCAAGA
ACCAGTGCGTGAACTTCAACTTCAATGGCCTTACCGGCACGGGCGTGCTGACCCCTAGCAGCAAAAGA
TTCCAGCCTTTTCAGCAGTTCGGCAGAGACGTGTCAGACTTCACCGATAGCGTGCGGGACCCCAAGAC
CTCTGAAATCCTGGACATCAGCCCATGTTCTTTCGGCGGAGTGAGCGTGATCACCCCTGGCACTAACG
CCTCCTCCGAGGTGGCCGTGCTGTACCAGGACGTTAATTGCACCGACGTGTCCACAGCCATCCACGCC
GACCAACTGACCCCTGCTTGGAGAATCTACTCTACCGGCAACAACGTTTTCCAGACACAGGCTGGATG
TCTGATCGGAGCCGAGCATGTGGACACCAGCTACGAGTGCGACATCCCCATCGGAGCCGGGATCTGCG
CCAGCTACCACACCGTGTCCCTGCTCCGGTCTACCAGCCAGAAGAGCATAGTGGCTTACACAATGAGC
CTGGGAGCCGACAGCAGCATCGCTTATAGCAACAACACCATCGCCATCCCTACCAACTTCTCTATCTC
CATCACAACTGAAGTCATGCCTGTCAGCATGGCCAAGACCAGCGTGGACTGCAACATGTACATCTGCG
GCGATTCTACCGAATGCGCCAACCTGCTGCTCCAGTACGGGTCCTTCTGCACCCAACTGAACAGGGCC
CTGTCCGGCATCGCCGCTGAACAGGACCGGAACACAAGAGAAGTGTTCGCCCAAGTGAAGCAAATGTA
CAAGACCCCCACCCTGAAGTACTTCGGCGGCTTCAACTTCAGCCAGATCCTGCCTGACCCTCTGAAAC
CTACAAAAAGAAGCTTCATCGAGGACCTGCTGTTTAACAAGGTGACCCTGGCCGACGCCGGCTTCATG
AAACAGTACGGCGAGTGTCTGGGAGACATCAACGCCAGAGACCTGATCTGCGCCCAAAAATTCAATGG
ACTCACAGTGCTGCCCCCCCTGCTGACTGATGATATGATCGCCGCCTACACTGCTGCTCTGGTGTCCG
GCACCGCCACAGCCGGCTGGACCTTTGGAGCCGGAGCCGCCCTGCAGATCCCCTTTGCCATGCAGATG
GCCTATCGGTTCAACGGGATCGGAGTTACACAGAACGTTCTGTACGAGAACCAGAAGCAAATCGCCAA
TCAGTTCAACAAGGCCATTAGCCAGATCCAGGAGAGCCTGACAACCACCAGCACCGCCCTGGGCAAGC
TGCAAGACGTAGTGAATCAGAACGCCCAGGCCCTGAATACCCTGGTGAAGCAACTGTCAAGCAATTTC
GGCGCTATCAGCAGCGTGCTGAACGACATCCTGAGCAGACTGGACCCTCCCGAAGCCGAGGTTCAGAT
CGACCGGCTGATCACAGGGAGACTGCAGTCCCTGCAAACATACGTGACCCAGCAACTGATCCGGGCCG
CCGAAATCAGAGCTAGTGCCAACCTGGCCGCCACCAAGATGAGCGAGTGCGTGCTGGGACAATCTAAG
AGAGTCGACTTCTGCGGCAAGGGCTATCACCTGATGAGCTTCCCCCAGGCCGCTCCTCATGGCGTGGT
CTTCCTGCATGTGACCTACGTGCCCTCCCAGGAACGGAACTTCACCACAGCCCCTGCCATCTGCCACG
AGGGAAAAGCCTACTTTCCCAGAGAGGGCGTGTTCGTGTTTAACGGCACATCTTGGTTCATCACACAG
AGAAACTTCTTCAGCCCTCAGATCATTACAACAGATAACACCTTCGTCAGCGGAAATTGTGACGTGGT
GATTGGCATCATCAACAACACCGTGTACGACCCTCTGCAGCCCGAACTGGACAGCTTCAAGGAGGAAC
TGGACAAGTACTTTAAGAACCACACATCTCCAGATGTGGACCTGGGCGACATCTCCGGAATCAACGCC
AGCGTGGTGAACATCCAGAAAGAAATCGACAGACTGAATGAGGTTGCTAAGAACCTGAACGAGTCTTT
GATCGATCTGCAGGAGCTGGGGAAGTACGAGCAGTACATCAAGTGGCCATGGTACGTGTGGCTGGGCT
TCATCGCCGGCCTGATCGCCATTGTGATGGTGACAATCCTGCTGTGCTGCATGACAAGCTGCTGTAGC
TGCCTGAAGGGCGCTTGTTCCTGTGGCTCTTGCTGCAAGTTCGATGAAGATGATAGCGAACCCGTGCT
GAAGGGCGTGAAGCTGCACTACACCTGA
SEQ ID NO: 33-SARS CoV-1 Spike Glycoprotein, mRNA Sequences
(Prefusion(2PP))
AUGUUCAUCUUCCUGCUGUUCCUGACUCUGACCAGCGGAUCUGAUCUGGACCGGUGCACCACCUUUGA
CGACGUGCAGGCCCCUAACUACACCCAGCACACCUCCAGCAUGAGAGGCGUGUACUACCCCGACGAGA
UUUUCAGAUCAGACACCCUGUACCUCACCCAGGACCUGUUCCUGCCUUUCUACAGCAACGUGACCGGC
UUUCACACCAUCAAUCACACCUUCGGCAACCCUGUGAUACCCUUCAAGGAUGGGAUCUACUUUGCCGC
CACAGAGAAAAGCAACGUGGUCCGGGGCUGGGUGUUUGGGUCUACAAUGAACAACAAGAGCCAGAGCG
UGAUCAUCAUCAACAAUAGCACCAACGUGGUCAUCAGAGCUUGUAAUUUCGAGCUGUGCGACAACCCA
UUCUUCGCCGUGUCCAAGCCUAUGGGCACCCAGACCCACACGAUGAUCUUCGAUAAUGCCUUCAAUUG
CACCUUCGAGUACAUCUCCGAUGCUUUUUCUCUCGACGUCUCUGAGAAGUCCGGCAAUUUCAAGCACC
UGAGAGAGUUCGUGUUCAAAAACAAAGACGGCUUCCUGUACGUGUACAAGGGCUACCAGCCAAUCGAC
GUGGUGCGGGACCUGCCUAGCGGCUUUAACACACUGAAGCCCAUCUUCAAGCUGCCUCUGGGCAUCAA
CAUCACAAACUUUAGAGCCAUCCUGACCGCUUUUAGCCCUGCCCAGGACAUCUGGGGCACCAGCGCCG
CUGCAUACUUCGUGGGCUACCUGAAACCCACCACAUUUAUGCUGAAGUACGACGAGAACGGCACAAUC
ACCGACGCCGUGGACUGCUCUCAGAACCCCCUGGCCGAGCUGAAAUGUUCCGUGAAGAGCUUUGAAAU
CGAUAAGGGCAUCUACCAGACCAGCAACUUUCGGGUGGUGCCUUCUGGAGACGUGGUGCGCUUCCCUA
ACAUUACAAACCUGUGUCCAUUCGGCGAAGUGUUUAACGCCACCAAGUUCCCUUCUGUGUACGCCUGG
GAGAGAAAGAAAAUCAGCAACUGCGUGGCCGAUUACAGCGUGCUGUAUAACAGUACAUUUUUCAGCAC
AUUCAAGUGUUACGGCGUUAGCGCCACAAAGCUUAAUGACUUGUGCUUCUCAAACGUGUACGCCGACA
GCUUCGUGGUCAAGGGCGACGACGUGCGGCAGAUUGCCCCUGGUCAGACCGGCGUGAUAGCCGAUUAC
AACUACAAGCUGCCCGACGAUUUCAUGGGCUGCGUGCUGGCCUGGAACACCAGAAAUAUCGACGCCAC
AAGCACCGGCAAUUACAACUACAAGUAUAGAUACCUGAGGCACGGCAAGCUGAGACCUUUCGAGAGAG
ACAUCAGCAACGUGCCAUUCAGCCCUGAUGGCAAGCCUUGCACCCCACCUGCCCUCAACUGCUACUGG
CCCCUGAACGACUACGGCUUCUACACCACAACAGGCAUUGGCUAUCAGCCUUACAGAGUUGUGGUUCU
GAGCUUCGAACUGCUGAACGCUCCUGCUACCGUUUGUGGCCCUAAGCUGAGCACCGACCUGAUCAAGA
ACCAGUGCGUGAACUUCAACUUCAAUGGCCUUACCGGCACGGGCGUGCUGACCCCUAGCAGCAAAAGA
UUCCAGCCUUUUCAGCAGUUCGGCAGAGACGUGUCAGACUUCACCGAUAGCGUGCGGGACCCCAAGAC
CUCUGAAAUCCUGGACAUCAGCCCAUGUUCUUUCGGCGGAGUGAGCGUGAUCACCCCUGGCACUAACG
CCUCCUCCGAGGUGGCCGUGCUGUACCAGGACGUUAAUUGCACCGACGUGUCCACAGCCAUCCACGCC
GACCAACUGACCCCUGCUUGGAGAAUCUACUCUACCGGCAACAACGUUUUCCAGACACAGGCUGGAUG
UCUGAUCGGAGCCGAGCAUGUGGACACCAGCUACGAGUGCGACAUCCCCAUCGGAGCCGGGAUCUGCG
CCAGCUACCACACCGUGUCCCUGCUCCGGUCUACCAGCCAGAAGAGCAUAGUGGCUUACACAAUGAGC
CUGGGAGCCGACAGCAGCAUCGCUUAUAGCAACAACACCAUCGCCAUCCCUACCAACUUCUCUAUCUC
CAUCACAACUGAAGUCAUGCCUGUCAGCAUGGCCAAGACCAGCGUGGACUGCAACAUGUACAUCUGCG
GCGAUUCUACCGAAUGCGCCAACCUGCUGCUCCAGUACGGGUCCUUCUGCACCCAACUGAACAGGGCC
CUGUCCGGCAUCGCCGCUGAACAGGACCGGAACACAAGAGAAGUGUUCGCCCAAGUGAAGCAAAUGUA
CAAGACCCCCACCCUGAAGUACUUCGGCGGCUUCAACUUCAGCCAGAUCCUGCCUGACCCUCUGAAAC
CUACAAAAAGAAGCUUCAUCGAGGACCUGCUGUUUAACAAGGUGACCCUGGCCGACGCCGGCUUCAUG
AAACAGUACGGCGAGUGUCUGGGAGACAUCAACGCCAGAGACCUGAUCUGCGCCCAAAAAUUCAAUGG
ACUCACAGUGCUGCCCCCCCUGCUGACUGAUGAUAUGAUCGCCGCCUACACUGCUGCUCUGGUGUCCG
GCACCGCCACAGCCGGCUGGACCUUUGGAGCCGGAGCCGCCCUGCAGAUCCCCUUUGCCAUGCAGAUG
GCCUAUCGGUUCAACGGGAUCGGAGUUACACAGAACGUUCUGUACGAGAACCAGAAGCAAAUCGCCAA
UCAGUUCAACAAGGCCAUUAGCCAGAUCCAGGAGAGCCUGACAACCACCAGCACCGCCCUGGGCAAGC
UGCAAGACGUAGUGAAUCAGAACGCCCAGGCCCUGAAUACCCUGGUGAAGCAACUGUCAAGCAAUUUC
GGCGCUAUCAGCAGCGUGCUGAACGACAUCCUGAGCAGACUGGACCCUCCCGAAGCCGAGGUUCAGAU
CGACCGGCUGAUCACAGGGAGACUGCAGUCCCUGCAAACAUACGUGACCCAGCAACUGAUCCGGGCCG
CCGAAAUCAGAGCUAGUGCCAACCUGGCCGCCACCAAGAUGAGCGAGUGCGUGCUGGGACAAUCUAAG
AGAGUCGACUUCUGCGGCAAGGGCUAUCACCUGAUGAGCUUCCCCCAGGCCGCUCCUCAUGGCGUGGU
CUUCCUGCAUGUGACCUACGUGCCCUCCCAGGAACGGAACUUCACCACAGCCCCUGCCAUCUGCCACG
AGGGAAAAGCCUACUUUCCCAGAGAGGGCGUGUUCGUGUUUAACGGCACAUCUUGGUUCAUCACACAG
AGAAACUUCUUCAGCCCUCAGAUCAUUACAACAGAUAACACCUUCGUCAGCGGAAAUUGUGACGUGGU
GAUUGGCAUCAUCAACAACACCGUGUACGACCCUCUGCAGCCCGAACUGGACAGCUUCAAGGAGGAAC
UGGACAAGUACUUUAAGAACCACACAUCUCCAGAUGUGGACCUGGGCGACAUCUCCGGAAUCAACGCC
AGCGUGGUGAACAUCCAGAAAGAAAUCGACAGACUGAAUGAGGUUGCUAAGAACCUGAACGAGUCUUU
GAUCGAUCUGCAGGAGCUGGGGAAGUACGAGCAGUACAUCAAGUGGCCAUGGUACGUGUGGCUGGGCU
UCAUCGCCGGCCUGAUCGCCAUUGUGAUGGUGACAAUCCUGCUGUGCUGCAUGACAAGCUGCUGUAGC
UGCCUGAAGGGCGCUUGUUCCUGUGGCUCUUGCUGCAAGUUCGAUGAAGAUGAUAGCGAACCCGUGCU
GAAGGGCGUGAAGCUGCACUACACCUGA

The VLPs described herein may be prepared as mRNA vaccines according to methods well known in the art. For example, in some embodiments, one or more segments of mRNA, e.g., such as those described above, is administered to a subject such that recipient cells express VLPs and/or polypeptides and/or proteins encoded by the mRNA. In some embodiments, recipient cells expressing such polypeptides produce VLPs comprising the polypeptides.

In accordance with embodiments described herein, a patient may be vaccinated with a single mRNA as described herein. In some embodiments, a single mRNA encodes more than one element of a VLP (e.g., more than one of structural polyprotein, viral tegument polypeptide, viral glycoprotein, etc.). For example, in some embodiments, an mRNA encodes two or more elements of a VLP. In some embodiments, a single mRNA encodes three or more elements of a VLP and/or antigen.

In some embodiments, subjects are vaccinated with mRNA encoding two or more elements of a VLP. For example, in some embodiments, in vivo patient cells and/or epithelial cell lines are transfected with a first mRNA encoding a Gag polypeptide and a second mRNA encoding a viral envelope glycoprotein. In some embodiments, a delivery vehicle such as a lipid nanoparticle (LNP) is loaded with mRNA that encodes all components of the VLP (structural and antigenic polypeptides). In some embodiments, the delivery vehicle or LNP has two or more mRNAs encoding different elements of the VLP and/or antigens.

In some embodiments, mRNAs encoding monovalent, bivalent, trivalent or more components of VLPs are admixed. For example, in some embodiments, monovalent and bivalent mRNAs encoding VLPs are admixed to form a trivalent VLP mRNA mixture. In some embodiments two bivalent mRNAs encoding VLPs are admixed to form a quadrivalent VLP mixture. In some embodiments, the mRNA encodes antigens from different pathogens. In some embodiments, the mRNA encodes antigens from different viruses.

Epstein-Barr virus (EBV) can enter B cells when virally encoded glycoprotein gp350 binds to host cell receptors. EBV encoded EBNA1 is involved in both latent and lytic stages of infection.

EBV-encoded nuclear antigen 1 (EBNA1) includes a unique glycine-alanine repeat domain that inhibits the endogenous presentation of cytotoxic T lymphocyte (CTL) epitopes through the class I pathway by blocking proteasome-dependent degradation of this antigen (Tellam et al., JBC Vol. 276, No. 36, Issue of September 7, pp. 33353-33360, 2001). Thus, the glycine-alanine repeat (GAr) domain of EBNA1 acts as a cis-inhibitor of MHC class I-restricted presentation. This immune evasion mechanism has been implicated in the pathogenesis of EBV-associated diseases.

Given that the primary targets of EBV are mainly B cells and epithelial cells, and that EBV envelope proteins are required for cellular entry, these proteins are attractive candidates for incorporation into vaccines. EBV gp350, gH, gL, gB and gp42 are involved in infection of B cells. EBV BMFR2, gH, gL and gB are involved in entry into epithelial cells. Cui et al., Frontiers in Immunology October 2021, Volume 12, Article 734471. Therefore, in some embodiments the mRNA encodes any combination of EBV gp350, gH, gL, gB, gp42, BMFR2 and EBNA1.

There are technologies that have been shown to successfully package and deliver mRNA in vivo to generate immune responses for prophylactic and therapeutic purposes. Strategies for delivery include vehicles such as liposomes, lipid nanoparticles, polymers, peptides, dendritic cells (in addition to free mRNA in solution).

Lipid-based delivery vehicles for mRNA have recently centered on lipid nanoparticles (LNPs). LNPs are generally nano-sized particles composed of synthetic or naturally occurring lipid materials. LNPs encapsulate mRNA thereby protecting against enzymatic degradation but also effectively delivering the mRNA into the cell cytosol through a series of endocytic mechanisms. Some embodiments described herein incorporate LNPs to deliver the mRNA in vitro and in vivo.

Polymer-based delivery vehicles include polyamines, dendrimers, and copolymers. These systems also protect mRNA from enzyme mediated degradation and provide intracellular delivery. To enhance safety, polymer-based vehicles may additionally incorporate lipid chains, hyperbranched groups, and biodegradable subunits. Some embodiments described herein incorporate polymer-based delivery vehicles to deliver the mRNA in vitro and in vivo.

Peptide-Based delivery of RNA can be accomplished using peptides and/or polypeptides as the primary carrier. Peptides for mRNA delivery are typically positively charged (and possibly containing many lysine and arginine residues). Protamine is an arginine-rich small protein that has been used to complex with RNA and used as an adjuvant for vaccination. Cationic cell-penetrating peptides (CCPs) can also complex with RNA. An example of a CCP is a “RALA” peptide (sequence: N-WEARLARALARALARHLARALARALRACEA-C) (SEQ ID NO: 34) which is amphipathic, arginine-rich, and positively charged (McCarthy et al. 2014). Some embodiments incorporate peptide-based delivery vehicles to deliver the mRNA in vitro and in vivo. Some embodiments incorporate CCP delivery vehicles to deliver the mRNA in vitro and in vivo.

Cationic Nanoemulsion (CNE) delivery combines nanoemulsion with cationic lipids. Cationic lipids in the squalene-based formulation create positively charged CNE particles that can absorb negatively charged nucleic acids to the outer shell of the CNE for RNA delivery. This structure similarly protects RNA from RNAse degradation. Some embodiments incorporate CNE delivery vehicles to deliver the mRNA in vitro and in vivo.

Naked mRNA vaccines are delivered without any additional carrier. The mRNA is simply dissolved in a solution and injected into the subject. Typically, the delivery route is via intramuscular injection. Exposure to RNAse, which is present in the bloodstream, and which would degrade the mRNA, can be ameliorated by local administration such as intramuscular, intranodal, intratracheal, intradermal, and intranasal routes. Naked mRNA vaccines are easily stored and rapidly prepared. If freeze-dried, the mRNA need only be dissolved in a buffer and administered. Naked mRNA also has inherent adjuvant properties. Some embodiments described herein deliver naked mRNA in vitro and in vivo.

Dendritic cell-based mRNA delivery, for example, using dendritic cells (DCs) derived from autologous primary human peripheral blood mononuclear cells (PBMCs) can elicit strong immune responses because DCs are professional antigen presenting cells (APCs). In addition to transfecting DCs with mRNA to antigens, additional maturation and activation signals can be provided for optimal immune response. Some embodiments described herein incorporate dendritic cell-based delivery of mRNA in vitro and in vivo.

Two or more antigens can be encoded by mRNA to broaden and strengthen the immune response. One challenge to using more than one antigen is that the in vivo expression level and immunostimulatory effect of each antigen may be different. It is also possible that the expression of one antigen may interfere or suppress the expression of a second antigen. The level of this effect in vivo is difficult to predict. Even if an antigen elicits a sufficient immune response when used alone, delivery of two or more antigens may lead to competition for epitope presentation and therefore a diminished response.

Lipid nanoparticles (LNPs) typically consist of four components-ionizable lipid, phospholipid, cholesterol, and PEGylated lipid, among which, the ionizable lipid plays a major role in protecting RNAs and facilitating their cytosolic transport. Ionizable lipids are positively charged at acidic pH to condense RNAs into LNPs but are neutral at physiological pH to minimize toxicity (Han et al., Nature Communications (2021) 12:7233).

LNPs usually contain at least one functional lipid that is essential for intracellular delivery of mRNA. Cationic or ionizable lipid materials often contain one or multiple amino groups. These lipids can be positively charged to encapsulate negatively charged RNA. The release of RNA from LNPs into the cytosol may involve disruption in endosomes. Delivery routes of LNPs are quite varied-including intramuscular (IM), intradermal (ID), subcutaneous (SC), intravenous (IV), and intranodal.

LNPs are the most clinically advanced non-viral delivery system for nucleic acids. LNPs are typically constituted of cationic ionizable lipid, cholesterol, helper lipid, stabilizer, and a nucleic acid payload. LNPs can be made by mixing an organic phase (e.g. containing an ionizable lipid, a helper lipid, cholesterol, a PEGylated lipid in ethanol) with an mRNA-containing aqueous phase in citrate or acetate buffer at acidic pH (Chaudhary N et al. Nat Rev Drug Discov. 2021 (11): 817-838. PMID: 34433919). Upon mixing, the ionizable lipid is protonated causing electrostatic attraction with the anionic mRNA. The result is spontaneous self-assembly of LNPs and encapsulation of the mRNA.

In some embodiments, microfluidic mixing is an effective means to formulate lipid nanoparticles (LNPs) because it is scalable and reproducible. An aqueous buffer containing mRNAs and an organic lipid mixture are rapidly mixed in a controlled and non-turbulent manner. This step facilitates self-assembly into stable nanostructures such that mRNAs are encapsulated in the interior core through interactions with ionizable lipids, cholesterol, helper lipids, and stabilizers.

After mRNA is encapsulated, the LNPs may be diluted, or buffer exchanged, with a suitable aqueous buffer to reduce the organic solvent content. Nanoparticle size and polydispersity index (PDI), which gives the size distribution of nanoparticles, can be determined by using a dynamic light scattering bioanalyzer. A high PDI indicates a broad or multimodal size distribution. The efficiency of RNA encapsulation can be measured using fluorescence-based, RNA-specific, assays.

LNP polishing exchanges the buffer and removes excess lipids and non-encapsulated mRNA to further increase the purity. This step can be achieved by centrifugal ultrafiltration based upon separation on molecular size. Molecules larger than the membrane pores will be retained, but not bound, at the surface of the membrane and concentrated during the ultrafiltration process. The retention properties of ultrafiltration membranes are expressed as molecular weight cut-off. At larger scales, centrifugal ultrafiltration can be replaced with tangential flow filtration systems. A final step may include sterile filtration of the mRNA-LNP.

In some embodiments, an organic solvent containing dissolved lipids and an aqueous solution containing nucleic acids are mixed. The two solutions do not immediately mix, but microscopic features engineered into a mixing channel cause the two fluids to intermingle in a controlled and reproducible way. Quickly, the two fluids are completely mixed, causing a change in solvent polarity that triggers the self-assembly of lipid nanoparticles loaded with nucleic acids. In one embodiment, the speed and ratio of fluid injection is varied to change the size of the lipid nanoparticles. Lipid nanoparticles mimic low-density lipoproteins, which allows them to be taken up by an endogenous cellular transport pathway to deliver nucleic acids to cells. Using pH-sensitive lipids allow lipid nanoparticles to release encapsulated nucleic acids into the cytoplasm when vesicle pH decreases.

In some embodiments, the ionizable lipid may be 1,1′-((2-(4-(2-((2-(bis(2-hydroxydodecyl)amino)ethyl)(2-hydroxydodecyl)amino)ethyl) piperazin-1-yl)ethyl)azanediyl)bis(dodecan-2-ol) (C12-200). The formula of C12-200 is illustrated below:

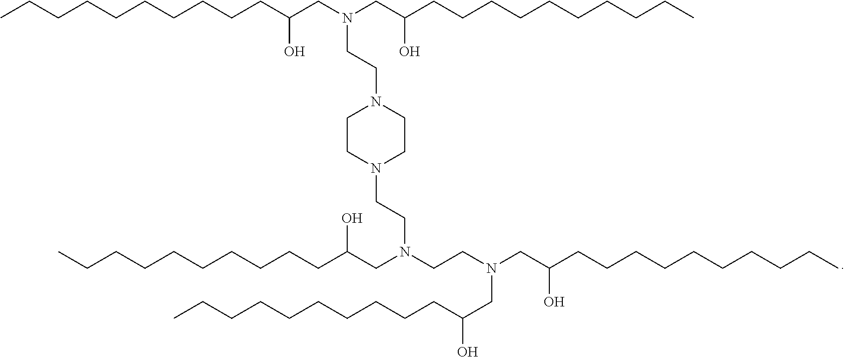 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, the phospholipid may be 1,2-dioleoyl-sn-glycero-3-phosphoethanolamine (DOPE).

In some embodiments, the PEGylated lipid may be 1,2-dimyristoyl-rac-glycero-3-methoxypolyethylene glycol-2000 (PEG-2000 DMG).

In particular embodiments, the LNP may include cholesterol and one or more of C12-200, DOPE, and PEG-2000 DMG. The C12-200, DOPE, cholesterol, and PEG-2000 DMG may be present in a molar ratio of 33.5±1.7:19±1:45.1±2.3:2.4=0.1.

The lipids may be present in the composition at a concentration of about 2 to about 6 mg/mL, for example a concentration of about 3 to about 5 mg/mL, such as about 4 mg/mL.

The composition may be a buffered solution that includes phosphate and sucrose and/or trehalose. For example, the composition may include about 8 mM to about 12 mM, such as about 10 mM, of phosphate; and/or the composition may include about 7 wt % to about 9 wt %, such as about 8 wt %, of sucrose, trehalose, or a combination thereof.

The composition may be at a pH of about 7.0 to about 7.4, such as a pH of about 7.2.

In a particular example, the lipid nanoparticle composition includes: C12:200, DOPE, cholesterol, and PEG2000-DMG in a molar ratio of 33.5:19:45.1:2.4, 10 mM of phosphate, and 8 wt % sucrose. In this particular example, the lipids are at a lipid concentration of 4 mg/mL and the composition is at a pH of 7.2.

LNPs according to the present disclosure may be used to encapsulate mRNA, such as the mRNA as described herein. The mRNA may be loaded into the LNPs using a citrate buffer, such as a 50 mM citrate buffer. The mRNA may be provided at a concentration from about 100 to about 300 g/mL, such as a concentration from about 175 to about 225 μg/mL, such as a concentration of about 200 g/mL.

mRNA-LNPs may be manufactured using known techniques, such as microfluidics, high-pressure homogenization, solvent displacement, or emulsification. In the examples discussed below, the authors of the present disclosure produced mRNA-LNPs by preparing an aqueous solution of mRNA in citrate buffer at pH 3.0; dissolving the lipids in ethanol; combining the mRNA and lipid solutions using PNI's NanoAssemblr® (Precision Nanosystems); buffer exchanging the resulting mRNA loaded LN solution with a phosphate-based buffer; and filter sterilizing the buffered solution. Filter sterilization was performed by passing the buffered solution through a 0.22 μm PES filter. Details of the specific lipids, amounts, concentrations, mRNA, and other features of the experiments are outlined in Tables 6, 7, and 8.

In some examples, the mRNA encodes polypeptides that constitute a virus-like particle (VLP).

In one example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO:1; and at least one additional polypeptide comprising a spike glycoprotein from a beta coronavirus, such as discussed above.

In another example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO:1; and at least one additional polypeptide comprising a protein expressed by a tumor cell or cancer cell against which an immunogenic effect is directed.

In yet another example, the polypeptides that constitute the VLP may include a first polypeptide comprising a gag protein found in murine leukemia virus (MLV) having at least 95% sequence identity, such as at least 97%, 98%, 99%, 99.5%, or 100% sequence identity, with the amino acid sequence of SEQ ID NO:1; and at least one additional polypeptide comprising a protein of a virus against which an immunogenic effect is directed, such as a protein of Epstein-Barr virus (EBV) or cytomegalovirus (CMV).

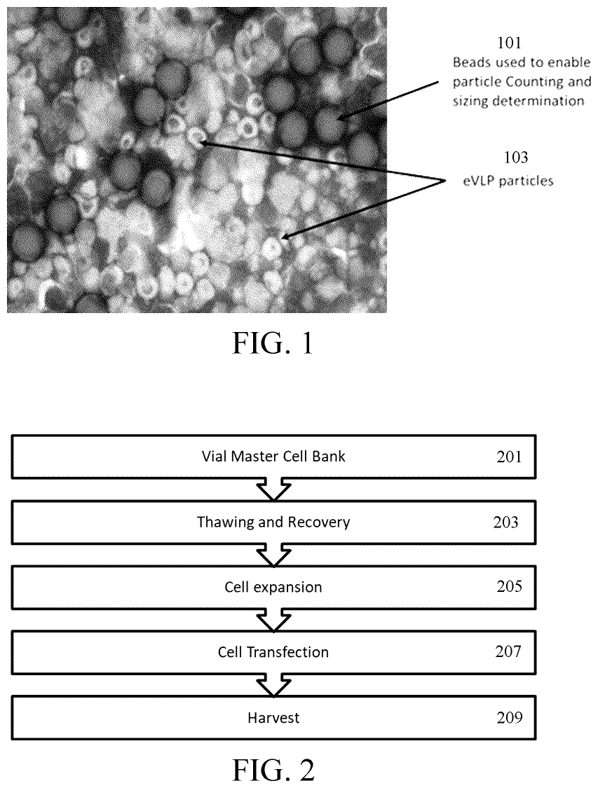

FIG. 1 is a negative stain transmission electron microscopic (nsTEM) image of eVLP particles produced in vitro after addition of an LNP formulation containing mRNA encoding for MMLV Gag. Beads (101) are present to enable particle counting and size determination of eVLP particles (103) formed. eVLP particles can be produced from mRNA or from Plasmid DNA. FIG. 1 demonstrates the production of eVLPs in vitro after addition of an LNP formulation containing mRNA encoding MMLV Gag.

### EXAMPLES

The following examples describe some exemplary modes of making and practicing certain compositions that are described herein. It should be understood that these examples are for illustrative purposes only and are not meant to limit the scope of the compositions and methods described herein.

### Example 1: Production of MERS mRNA LNP Formulation for In Vivo Animal Studies

For production of LNP-mRNA test articles, mRNA was dissolved in citrate buffer pH 3.0. Lipids and reaction components were dissolved in Ethanol: SM-102 (2.29 mg/mL), DSPC (0.51 mg/mL), Cholesterol (0.96 mg/mL), DMG-PEG 2000 (0.24 mg/mL). The aqueous and organic (ethanol) phases were transferred into separate BD™ syringes which were then combined using PNI's NanoAssemblr® (Precision Nanosystems). The resulting mRNA loaded LNP solution was buffer exchanged into 10 mM Phosphate 8% Sucrose buffer (pH7.2) and sterilized by filtration through a 0.22 μm pore membrane. The physiochemical properties of the test articles are presented in Table 1. The encapsulation efficiency is noted as “EE %” which may be calculated based on average OD values.

TABLE 1
Physiochemical properties of test articles used to evaluate mRNA formulations against MERS.

| | mRNA conc. in LNP or | | | MERS-CoV | | | | | | |
|---|---|---|---|---|---|---|---|---|---|---|
| | Antigen conc. for eVLP | | | Spike | | | | | | |
| Test Article | MERS- | | Total | mRNA:Gag | | | Zeta | | Zeta | Encapsulation |
| Formulation | CoV Spike | Gag | quantity | mRNA | Lipid:mRNA | Osmolality | Size | | Potential | efficiency |
| Type | (μg/mL) | (μg/mL) | (μg/mL) | ratio | w/w ratio | (mOsm/kg) | (nm) | PDI | (mV) | (%) |
| Test Article | 25 | NA | 25 | NA | 160:1 | 327 | 129.0 | 0.101 | 2.64 | 95.7 |
| 1 | | | | | | | | | | |
| mRNA-LNP | | | | | | | | | | |
| Test Article | 25 | 225 | 250 | 1:9 | 16:1 | 333 | 88.02 | 0.086 | −0.866 | 96.5 |
| 2 | | | | | | | | | | |
| mRNA-LNP | | | | | | | | | | |
| Test Article | 1 | NA | 1 | NA | NA | 284 | 2061 | 0.109 | −32.7 | NA |
| 3 | | | | | | | | | | |
| eVLP | | | | | | | | | | |

Six-to eight-week-old C57/BL6 mice (n=10/group) were immunized intramuscularly twice on days 0 and 28 with the test articles listed in Table 2. MERS Spike eVLPs were produced as previously described to compare the immunogenicity of mRNA-encoded eVLPs to biologically produced eVLPs (Fluckiger A-C, 2021). Biologically produced eVLPs expressed 0.1 μg Spike protein and were formulated with aluminum phosphate containing 125 μg of alum content.

TABLE 2
T cell responses induced with mRNA formulations against MERS

| | | | CD8+ T cells | | CD4+ T cells | |
|---|---|---|---|---|---|---|
| | MERS mRNA | Gag mRNA | | IFN-γ+ | | IFN-γ+ |
| Test Article | (μg/ml) | (μg/ml) | IFN-γ+ | TNF-α+ | IFN-γ+ | TNF-α+ |
| TA1: MERS Spike | 1.25 | N/A | 7.19 ± | 3.87 ± | 0.23 ± | 0.21 ± |
| mRNA | | | 0.63 | 0.34 | 0.06 | 0.04 |
| TA2: MERS Spike + | 1.25 | 11.25 | 6.92 ± | 6.50 ± | 0.25 ± | 0.34 ± |
| Gag mRNAs | | | 0.78 | 1.29 | 0.05 | 0.05 |

| | | | | | |
|---|---|---|---|---|---|
| MERS Spike eVLPs | Not applicable | 1.27 ± | 0.04 ± | 0.08 ± | 0.04 ± |

| | | | |
|---|---|---|---|
| 0.34 | 0.01 | 0.03 | 0.01 |

Fourteen days after the second vaccination, spleens were harvested from the mice to assess induction of MERS-specific CD4+ and CD8+ T cells using multiparameter flow cytometry. Briefly, the spleens from individual mice were processed to produce single cell suspensions. Erythrocytes were lysed using a commercially available RBC lysis buffer (BioLegend). Splenocytes were then re-suspended at 2×10⁶ splenocytes/mL (2× the final stimulation concentration) in R10 (RPMI+10% FBS). 500 μl of the splenocyte suspension and 500 μl of the stimulants at 2× final concentration was then aliquoted into 5 mL polypropylene tubes. The final volume per tube was 1 mL. The following stimulants were used: MERS Spike pepmix 1, MERS spike pepmix 2, Gp350 pepmix, EBNA-1 pepmix, R10 (negative control) PMA (20 ng/ml)+Ionomycin (1 μg/ml) (positive control). All pepmixes were purchased from JPT Peptides (Berlin, Germany) and used at a final concentration of 1 μg/ml. The tubes were then placed into a humid incubator at 37° C. with 5% CO₂. After 2 hours 50 μl of R10 containing Brefeldin A (BD) (1 μL/mL) and Monensin (BD) (0.7 μL/mL) was added to each tube. The tubes were then incubated for a further 18-20 hours. After the incubation the splenocytes were washed and surface stained for CD3, CD4, CD8 and dead cells. The cells were then fixed and permeabilized using the BD Cytofix/Cytoperm™ Fixation/Permeabilization Kit. The splenocytes were then intracellular stained for IFN-γ and TNF-α. The stains for the MERS study were: CD4 FITC, CD8a PE-Cy7, CD3 BV605, FVS510, IFN-γ PE and TNF-α BB700. The stains for the gp350/EBNA-1 study were: CD4 FITC, CD8a PE-Cy7, CD3 BV650, FVS510, IFN-γ PE and TNF-α PerCP-Cy5.5 (all flow stains and buffers were purchased from BD Biosciences). The stained cells were then acquired on a BD Celesta flow cytometer and the data analyzed with Flowjo™ V10.8.1.

Immunization of mice with biologically produced MERS Spike eVLPs induced a CD8+ T cell response (1.27%) and marginal CD4+ T cell response (0.08%). While CD8+ T cells could be detected that secreted IFN-γ, there were very few polyfunctional T cells that secreted both IFN-γ and TNF-α. Immunization with an LNP formulation containing mRNA encoding for the MERS Spike protein induced a significantly stronger CD8+ T cell response, and polyfunctional CD8+ T cells could now be detected (3.87%), though they were significantly lower in frequency than those secreting only IFN-γ (7.19%). Notably, immunization with an LNP formulation containing mRNAs encoding for both the MERS Spike and MMLV Gag proteins induced polyfunctional CD8+ T cells that were as frequent as those secreting only IFN-γ (6.50% and 6.92%, respectively).

### Example 2

Production of EBV mRNA LNP Formulation for In Vivo Animal Studies

LNP-mRNA test articles were prepared as described in Example 1 and the physiochemical properties of the test articles are presented in Table 3. The encapsulation efficiency (EE %) is reported based on a calculation of average OD values.

TABLE 3
Physiochemical properties of test articles used to evaluate mRNA formulations against EBV

| | mRNA conc in LNP | | | gp350vsvG:Gag- | | | | | | |
|---|---|---|---|---|---|---|---|---|---|---|
| | | Gag- | | EBNA1 | | | | | | |
| Test Article | | EBNA1 | Total | Delta Gar | Lipid:mRNA | | Zeta | | Zeta | Encapsulation |
| Formulation | gp350vsvG | Delta Gar | quantity | mRNA | w/w | Osmolality | Size | | potential | efficiency |
| Type | (μg/ mL) | (μg/ mL) | (μg/mL) | weight ratio | ratio | (mOsm/kg) | (nm) | PDI | (mV) | (%) |
| Test Article | 25 | NA | 25 | NA | 160:1 | 269 | 92.48 | 0.057 | +3.34 | 93.9 |
| 1 | | | | | | | | | | |
| mRNA- | | | | | | | | | | |
| LNP | | | | | | | | | | |
| 4 | 25 | 225 | 250 | 1:9 | 16:1 | 273 | 123.4 | 0.067 | +2.19 | 89.6 |
| mRNA- | | | | | | | | | | |
| LNP | | | | | | | | | | |

Six-to eight-week-old C57/BL6 mice (n=10/group) were immunized intramuscularly twice on days 0 and 28 with the test articles listed in Table 4. Fourteen days after the second vaccination, spleens were harvested from the mice to assess induction of EBV gp350-specific CD4+ and CD8+ T cells using multiparameter flow cytometry, described above in Example 1.

TABLE 4
T cell responses induced with mRNA formulations against EBV

| | | | CD8+ T cells | | CD4+ T cells | |
|---|---|---|---|---|---|---|
| | gp350 mRNA | Gag mRNA | | IFN-γ+ | | IFN-γ+ |
| Test Article | (μg/ml) | (μg/ml) | IFN-γ+ | TNF-α+ | IFN-γ+ | TNF-α+ |
| 1 | 1.25 | N/A | 0.14 ± | 0.04 ± | 0.45 ± | 0.15 ± |
| | | | 0.10 | 0.03 | 0.22 | 0.06 |
| 4 | 1.25 | 11.25 | 0.18 ± | 0.17 ± | 0.19 ± | 0.36 ± |
| | | | 0.13 | 0.18 | 0.1 | 0.20 |

In contrast to T cell responses elicited against the MERS Spike protein, a predominantly CD4+ T cell response was directed against the EBV gp350 glycoprotein, with marginal CD8+ T cell responses. However, like what was observed against the MERS Spike protein, immunization of mice with an LNP formulation containing mRNA encoding for the EBV gp350 glycoprotein induced CD4+ T cells that secreted IFN-γ, but there were 3-fold fewer polyfunctional T cells that secreted both IFN-γ and TNF-α (0.45% vs. 0.15%, respectively). Notably, immunization with an LNP formulation containing mRNAs encoding for both the EBV gp350 glycoprotein and MMLV Gag fused with a truncated EBV EBNA1 structural protein induced polyfunctional CD4+ T cells that were approximately 2-fold higher than those secreting only IFN-γ (0.36% and 0.19%, respectively). Similar results were obtained among CD8+ T cells, though overall frequencies were lower.

Based on these data, the relative magnitude of the CD4+ versus CD8+ T cell response is influenced by the viral protein in question (e.g. MERS Spike protein or EBV gp350 glycoprotein). Nevertheless, regardless of the nature of the response (CD4+ or CD8+), mRNA-encoded eVLP expression of surface antigens (e.g. MERS Spike protein, EBV gp350 glycoprotein) induced higher frequencies of polyfunctional T cells than did immunization with mRNA-encoded antigens alone (without eVLP particle formation).

### Example 3

Production of EBV mRNA LNP Formulation for In Vivo Animal Studies

LNP-mRNA test articles were prepared as described in Example 1 and the physiochemical properties of the test articles are presented in Table 5. The encapsulation efficiency (EE %) is reported based on RIBOGREEN® assay.

TABLE 5
Physiochemical properties of test articles used to evaluate mRNA formulations against EBV (28B643)

| | mRNA conc. in LNP | | | | | | | | | |
|---|---|---|---|---|---|---|---|---|---|---|
| | Gp350 | Gag- | | gp350:Gag- | | | | | | |
| Test Article | Full | EBNA1 | Total | EBNA1 | Lipid:mRNA | Osmo. | Particle | | Zeta | Encapsulation |
| Formulation | Length | Δgar | qty. | Δgar | w/w | (mmol/ | size | | Potential | efficiency |
| Type | (μg/mL) | (μg/mL) | (μg/mL) | ratio | ratio | kg) | (nm) | PDI | (mV) | (%) |
| Test Article | 20 | 180 | 200 | 1:9 | 20:1 | 287 | 120.7 | 0.069 | 4.28 | 82.5 |
| 1 * | | | | | | | | | | |
| mRNA- | | | | | | | | | | |
| LNP | | | | | | | | | | |
| Test Article | 20 | 180 | 200 | 1:9 | 20:1 | 311 | 142.4 | 0.074 | 4.53 | 83.9 |
| 2 ** | | | | | | | | | | |
| mRNA- | | | | | | | | | | |
| LNP | | | | | | | | | | |

* SM-102:DSPC:cholesterol:PEG-2000DMG (Lipid Molar Ratio: 50:10:38.5:1.5)
** C12-200:DOPE:cholesterol:PEG-2000DMG (Lipid Molar Ratio: 33.5:19:45.1:2.4)

Six-to eight-week-old C57/BL6 mice (n=8/group) were immunized intramuscularly twice on days 0 and 28 with the test articles listed in Table 6. Fourteen days after the first vaccination and eleven to thirteen days after the second vaccination, blood samples were collected from the mice to assess induction of EBV gp350-specific total IgG titers using ELISA procedure. Both LNP formulations induced robust antibody responses, with no statistically significant differences between antibody titers elicited by the two LNP formulations.

TABLE 6
Antibody responses induced with mRNA
formulations against EBV gp350

| | | | Anti-gp350 Total IgG (GMT) | |
|---|---|---|---|---|
| Test | gp350 mRNA | Gag mRNA | After First | After Second |
| Article | (μg/dose) | (μg/dose) | Vaccination | Vaccination |
| 1 | 1.00 | 9.00 | 8,294 | 5,699,882 |
| 2 | 1.00 | 9.00 | 13,909 | 4,019,215 |

### Summary of Sequences

Sequences noted herein are provided in Table 7. The sequences pertain to exemplified embodiments but are not intended to be limiting. SEQ ID Nos 26 to 29 correspond to the sequences used in Study Design 28CH31D2, and SEQ ID Nos 30 to 33 correspond to the sequences used in Study Design 28CH42A.

TABLE 7
Brief Description of Sequences

| SEQ ID | |
|---|---|
| NO: | Description of Sequence |
| 1 | MMLV-Gag Amino Acid Sequence |
| 2 | MMLV-Gag Nucleotide Sequence |
| 3 | Codon Optimized MMLV-Gag Nucleotide Sequence |
| 4 | MMLV-Gag mRNA Sequence (codon optimized) |
| 5 | MMLV-Gag/EBV EBNA1 Amino Acid Sequence |
| 6 | MMLV-Gag/EBV EBNA1 Nucleotide Sequence |
| 7 | MMLV-Gag/EBV EBNA1 Codon Optimized Nucleotide Sequence |
| 8 | MMLV-Gag/EBV EBNA1 mRNA Sequence |
| 9 | MMLV-Gag/EBV gp350 Amino Acid Sequence Gag fused with gp350[22-434] |
| 10 | MMLV-Gag/EBV gp350 Nucleotide Sequence |
| 11 | MMLV-Gag/EBV gp350 Codon Optimized Nucleotide Sequence |
| 12 | MMLV-Gag/EBV gp350 mRNA Sequence |
| 13 | EBV gp350 Glycoprotein, Amino Acid Sequence |
| 14 | EBV gp350 Glycoprotein, Nucleotide Sequence |
| 15 | EBV gp350 Glycoprotein, Nucleotide Sequence, Codon Optimized For Expression In |
| | Human Cell |
| 16 | EBV gp350 Glycoprotein mRNA Sequence |
| 17 | MERS-CoV Spike Glycoprotein, Amino Acid Sequence |
| 18 | MERS-CoV Spike Glycoprotein, Nucleotide Sequence |
| 19 | MERS-CoV Spike Glycoprotein, Nucleotide Sequence, Codon Optimized For Expression |
| | In Human Cells |
| 20 | MERS-CoV Spike Glycoprotein mRNA Sequence |
| 21 | HCMV gB-G Amino Acid Sequence |
| 22 | HCMV gB-G Nucleotide Sequence |
| 23 | HCMV (gB-G) Codon Optimized Nucleotide Sequence |
| 24 | HCMV (gB-G) mRNA Sequence |
| 25 | EGFP mRNA sequence |
| 26 | SARS CoV 2 Spike Glycoprotein, Amino Acid Sequences (COVID-v4 FURIN |
| | CLEAVAGE Mod. & VSV-G TMCyt SWAP) |
| 27 | SARS CoV 2 Spike Glycoprotein, Nucleotide Sequences (COVID-v4 FURIN |
| | CLEAVAGE Mod. & VSV-G TMCyt SWAP) |
| 28 | SARS CoV 2 Spike Glycoprotein, Codon Optimized Sequences (COVID-v4 FURIN |
| | CLEAVAGE Mod. & VSV-G TMCyt SWAP) |
| 29 | SARS CoV 2 Spike Glycoprotein, mRNA Sequences (COVID-v4 FURIN CLEAVAGE |
| | Mod. & VSV-G TMCyt SWAP) |
| 30 | SARS CoV-1 Spike Glycoprotein, Amino Acid Sequences (Prefusion(2PP)) |
| 31 | SARS CoV-1 Spike Glycoprotein, Nucleotide Sequences (Prefusion(2PP)) |
| 32 | SARS CoV-1 Spike Glycoprotein, Codon Optimized Sequences (Prefusion(2PP)) |
| 33 | SARS CoV-1 Spike Glycoprotein, mRNA Sequences (Prefusion(2PP)) |
| 34 | Cationic cell-penetrating peptide (CCP) “RALA” |

### Example 4

### Exemplary LNP Formulations According to the Present Disclosure, and Comparative Formulations

LNP formulations according to Tables 8 and 9 were prepared as discussed above. An aqueous solution of mRNA in citrate buffer at pH 3.0 was prepared. The lipids were dissolved in 100% ethanol. The mRNA and lipid solutions were combined using PNI's NanoAssemblr® (Precision Nanosystems). The resulting mRNA loaded LNP solution was buffer exchanged with a phosphate-based buffer. The buffered solution was filter sterilized by passing the buffered solution through a 0.22 μm PES filter. The features of the different test articles (TAs) are outlined in the following tables.

In Tables 8 and 9, the Test Articles (TAs) identified by * and ** identify LNP formulations that include C12-200, DOPE, cholesterol, and PEG-2000 DMG in a molar ratio of 33.5=1.7:19±1:45.1±2.3:2.4±0.1. The TAs identified by ** also identify LNP formulations that include about 8 mM to about 12 mM of phosphate, and about 7 wt % to about 9 wt % of sucrose; and that are at a pH of about 7.0 to about 7.4.

The LNP formulations of Tables 8 and 9 were prepared using a total of 200 μg/mL of mRNA. The formulations included one or two of: SARS-COV-2 (SEQ ID NO: 29) Gag (SEQ ID NO: 4), EGFP, and gp350 (SEQ ID NO: 16). The EGFP mRNA was obtained from TriLink BioTechnologies and had a sequence according to SEQ ID NO: 25.

The resulting LNP formulations were characterized using the methods discussed above. The results are summarized in Table 10.

LNP formulations were also tested for transfection efficiency. The results are summarized below in Table 11.

TABLE 8

| | | | | | mRNA (ug/mL) | | | | | |
|---|---|---|---|---|---|---|---|---|---|---|
| Study | | | Molar lipid | Lipid | SARS- | | | | mRNA | |
| Design | TA | lipid composition | ratio | conc. | CoV-2 | Gag | EGFP | gp350 | solution | Buffer |
| 28CH26P2 | 1** | C12- | 33.5:19:45.1:2.4 | 4 | x | 100 | x | 100 | 50 mM citrate | 10 mM |
| | | 200:DOPE:cholesterol:PEG- | | mg/mL | | | | | buffer | phosphate, |
| | | 2000DMG | | | | | | | | 8% sucrose, |
| | | | | | | | | | | pH 7.2 |
| | 2* | | | | | | | | | 10 mM |
| | | | | | | | | | | phosphate, |
| | | | | | | | | | | 8% mannitol, |
| | | | | | | | | | | pH 7.2 |
| | 3* | | | | | | | | | 10 mM |
| | | | | | | | | | | phosphate, |
| | | | | | | | | | | 8% trehalose, |
| | | | | | | | | | | pH 7.2 |
| | 4* | | | | | | | | | TRIS with |
| | | | | | | | | | | 8.7% sucrose, |
| | | | | | | | | | | pH 7.2 |
| | 5* | | | | | | | | | PBS |
| | 6* | | | | | | | | | PBS with |
| | | | | | | | | | | 2% sucrose, |
| | | | | | | | | | | pH 7.2 |
| | 7 | | 35:16:46.5:2.5 | | | | | | 10 mM citrate | 10 mM |
| | | | | | | | | | buffer | phosphate, |
| | | | | | | | | | | 8% sucrose, |
| | | | | | | | | | | pH 7.2 |
| | 8 | | | | | | | | | PBS |
| | 9^{a} | SM- | 50:10:38.5:1.5 | | | | | | 50 mM citrate | 10 mM |
| | | 102:DSPC:cholesterol:PEG- | | | | | | | buffer | phosphate, |
| | | 2000DMG | | | | | | | | 8% sucrose, |
| | | | | | | | | | | pH 7.2 |
| 28CH26P4 | 1** | C12- | 33.5:19:45.1:2.4 | 4 | x | 100 | x | 100 | 50 mM citrate | 10 mM |
| | | 200:DOPE:cholesterol:PEG- | | mg/mL | | | | | buffer | phosphate, |
| | 2** | 2000DMG | | | | | | | 10 mM citrate | 8% sucrose, |
| | | | | | | | | | buffer | pH 7.2 |
| | 3 | | 35:16:46.5:2.5 | | | | | | 50 mM citrate | |
| | | | | | | | | | buffer | |
| | 4 | | | | | | | | 10 mM citrate | |
| | | | | | | | | | buffer | |
| | 5 | | | | | | | | 50 mM citrate | PBS |
| | | | | | | | | | buffer | |
| | 6^{b} | | | | | | | | 10 mM citrate | PBS |
| | | | | | | | | | buffer | |
| | 7^{c} | SM- | 50:10:38.5:1.5 | | | | | | 50 mM citrate | 10 mM |
| | | 102:DSPC:cholesterol:PEG- | | | | | | | buffer | phosphate, |
| | | 2000DMG | | | | | | | | 8% sucrose, |
| | | | | | | | | | | pH 7.2 |

^{a}TA1 of 28CH26N2;
^{b}TA8 of 28CH26P1;
^{c}TA1 of 28CH26N2

TABLE 9

| | | | | | mRNA (ug/mL) | | | | | |
|---|---|---|---|---|---|---|---|---|---|---|
| Study | | | Molar lipid | Lipid | SARS- | | | | mRNA | |
| Design | TA | lipid composition | ratio | conc. | CoV-2 | Gag | EGFP | gp350 | solution | Buffer |
| 28CH26N3 | 1** | C12- | 33.5:19:45.1:2.4 | 4 | x | 100 | x | 100 | 50 mM | 10 mM |
| | 2 | 200:DOPE:cholesterol:PEG- | 38.2:12.9:47.1:1.8 | mg/mL | x | 100 | x | 100 | citrate | phosphate, |
| | | 2000DMG | | | | | | | buffer | 8% sucrose, |
| | | | | | | | | | | pH 7.2 |
| | 3 | C12- | 33.8:18.1:45.7:2.4 | | x | 100 | x | 100 | | |
| | | 200:DSPC:cholesterol:PEG- | | | | | | | | |
| | | 2000DMG | | | | | | | | |
| | 4^{d} | C12- | 35:16:46.5:2.5 | | x | 100 | x | 100 | 10 mM | PBS |
| | | 200:DOPE:cholesterol:PEG- | | | | | | | citrate | |
| | | 2000DMG | | | | | | | buffer | |
| 28CH26M | 1** | C12- | 33.5:19:45.1:2.4 | 4 | x | x | 200 | x | 50 mM | 10 mM |
| | 2** | 200:DOPE:cholesterol:PEG- | | mg/mL | x | 200 | x | x | citrate | phosphate, |
| | 3 | 2000DMG | 38.2:12.8:47.1:1.8 | | x | x | 200 | x | buffer | 8% sucrose, |
| | 4 | | | | x | 200 | x | x | | pH 7.2 |
| | 5^{e} | | | | x | 200 | x | x | | |
| | 6 | | 55:5:38.5:1.5 | | x | 200 | x | x | | |
| | 7^{e} | | | | x | 200 | x | x | | |
| 28CH31E2 | 1 | Coatsome | 33.5:19:45.1:2.4 | 4 | x | 100 | x | 100 | 50 mM | 10 mM |
| | | SS- | | mg/mL | | | | | citrate | phosphate, |
| | | EC:DOPE:cholesterol:PEG- | | | | | | | buffer | 8% sucrose, |
| | | 2000DMG | | | | | | | | pH 7.2 |
| | 2 | 306Oi10- | 33.5:19:45.1:2.4 | | | | | | | |
| | | EC:DOPE:cholesterol:PEG- | | | | | | | | |
| | | 2000DMG | | | | | | | | |
| | 3** | C12- | 33.5:19:45.1:2.4 | | | | | | | |
| | | 200:DOPE:cholesterol:PEG- | | | | | | | | |
| | | 2000DMG | | | | | | | | |
| | 4 | 306Oi10- | 41.6:11.7:44.9:1.8 | | | | | | | |
| | | EC:DOPE:cholesterol:PEG- | | | | | | | | |
| | | 2000DMG | | | | | | | | |
| | 5 | SM- | 50:10:38.5:1.5 | | | | | | | |
| | | 102:DSPC:cholesterol:PEG- | | | | | | | | |
| | | 2000DMG | | | | | | | | |

^{d}TA4 transfection data from 28CH260
^{e}Lipid is dissolved in 90% Ethanol + 10% 50 mM citrate buffer instead of only Ethanol.

TABLE 10

| | Phys-chem | | | RIBOGREEN ® |
|---|---|---|---|---|
| | Particle Size | | | assay |
| TA# | (nm) | PDI | ZP (mV) | EE (%) |

Study Design: 28CH26P2

| | | | | |
|---|---|---|---|---|
| 1 | 108.3 | 0.070 | 3.06 | 86.6 |
| 2 | 244.7 | 0.382 | 12.1 | 91.9 |
| 3 | 98.73 | 0.110 | 7.72 | 78.8 |
| 4 | 131.8 | 0.097 | 12.1 | 91.7 |
| 5 | 910.7 | 0.810 | −1.64 | 79.7 |
| 6 | 90.32 | 0.116 | 14.8 | 90.1 |
| 7 | 111.3 | 0.098 | −5.41 | 46.7 |
| 8 | 316.1 | 0.443 | −13.6 | 30.3 |
| 9 | 93.7 | 0.051 | −1.11 | 97.0 |

Study Design: 28CH26P4

| | | | | |
|---|---|---|---|---|
| 1 | 124.6 | 0.113 | 0.933 | 82.8 |
| 2 | 113.6 | 0.069 | −3.12 | 59.3 |
| 3 | 129.8 | 0.096 | 0.564 | 81.1 |
| 4 | 105.2 | 0.097 | −7.31 | 49.7 |
| 5 | 1762 | 0.953 | −7.32 | 55.4 |
| 6 | 316.1 | 0.443 | −13.6 | 30.3 |
| 7 | 93.7 | 0.051 | −1.11 | 97.0 |

Study Design: 28CH26N3

| | | | | |
|---|---|---|---|---|
| 1 | 113.9 | 0.088 | 5.43 | 75.4 |
| 2 | 136.3 | 0.07 | 2.83 | 75.0 |
| 3 | 102.9 | 0.107 | 2.32 | 78.6 |
| 4 | 230.8 | 0.313 | −7.82 | 15.0 |

Study Design: 28CH26M

| | | | | |
|---|---|---|---|---|
| 1 | 138.5 | 0.059 | 3.39 | 89.4 |
| 2 | 116.3 | 0.078 | 0.722 | 78.3 |
| 3 | 141.8 | 0.069 | −0.868 | 74.2 |
| 4 | 134.7 | 0.076 | −0.475 | 77.4 |
| 5 | 136.5 | 0.057 | −0.154 | 75.3 |
| 6 | 117.7 | 0.105 | −25.90 | 31.2 |
| 7 | 122.6 | 0.089 | −29.30 | 42.4 |

Study Design: 28CH31E2

| | | | | |
|---|---|---|---|---|
| 1 | 134.4 | 0.201 | −9.68 | 80.5 |
| 2 | 117.9 | 0.081 | −13.1 | 90.6 |
| 3 | 93.02 | 0.144 | −3.57 | 75.8 |
| 4 | 183.4 | 0.193 | −24.1 | 89.1 |
| 5 | 135.4 | 0.109 | −0.782 | 85.2 |

TABLE 11

| | | |
|---|---|---|
| | Median Fluorescence | |
| TA | Intensity | % of gp350+ cells |

Study Design: 28CH26P2

| | | |
|---|---|---|
| 1 | 2978 | 68.8% |
| 2 | 2888 | 67.9% |
| 3 | 1669 | 43.5% |
| 4 | 2156 | 52.4% |
| 5 | 839 | 10.1% |
| 6 | 3005 | 69.6% |
| 7 | 1332 | 30.1% |
| 8 | 410 | 0.13% |
| 9 | 1519 | 37.4% |

Study Design: 28CH26P4

| | | |
|---|---|---|
| 1 | 15193 | 94.7% |
| 2 | 9562 | 88.4% |
| 3 | 12238 | 91.5% |
| 4 | 5946 | 82.0% |
| 5 | 4227 | 71.9% |
| 6 | 645 | 1.47% |
| 7 | 7726 | 87.3% |

Study Design: 28CH26N3

| | | |
|---|---|---|
| 1 | 1846 | 90.3% |
| 2 | 1438 | 90.1% |
| 3 | 137 | 15.8% |
| 4 | 563 | 1.74% |

Study Design: 28CH31E2

| | | |
|---|---|---|
| 1 | 151 | 0.47% |
| 2 | 718 | 58.7% |
| 3 | 1818 | 77.1% |
| 4 | 1239 | 69.4% |
| 5 | 2978 | 89.5% |

### ANALYTICAL METHODS

Physiochemical Analysis. For mRNA-LNP-related studies, various parameters—such as pH, osmolality, Zeta Size (ZS), and Zeta Potential (ZP)—were assessed for each Test Article. pH measurements were executed directly on the Test Articles using a Thermo Scientific Orion™ 350 pH meter equipped with a PerpHecT™ ROSS™ Combination pH Micro Electrode, adhering to SOP-GEN-022 Calibration and Operation of pH Meters. Osmolality evaluations were conducted utilizing the Wascor VAPRO® 5520 vapor pressure osmometer, following SOP-FORM-007 Osmometer guidelines. The determination of Zeta Potential and Particle Size Analysis of eVLPs was performed according to SOP-FORM-023, SOP-FORM-019 and SOP-FORM-012. The NanoZeta Sizer (Model No. ZEN3600) facilitated the analysis of nanosize populations within the samples using dynamic light scattering. To ensure accurate analysis, all samples were diluted (20-50×) in 0.1 μm filtered Water for Injection (WFI) and gently resuspended by pipette for 1 minute before undergoing testing in the instrumentation.

RIBOGREEN® Assay. The Quant-iT™ RIBOGREEN® Assay Kit from Thermo Fisher Scientific (R11490) was employed to quantify mRNA in the formulation (RIBOGREEN® Assay by Precision Nanosystems; Document ID: ribogreenassay-DC-0621). Other requirements include TRITON™ X-100 (Sigma Aldrich, X100-100 ML), RNase-free water, RNase-Free Filter Pipette Tips in various sizes (10, 20, 200, 300, and 1000 μL), Pipette basins, a 96-well clear plate, Mg2+/Ca2+ free PBS 1×, TE Buffer 20×, 18 Gauge Needles, a Plate Reader, a Multichannel Pipette (10-300 μL), and Micropipettes in sizes of 10, 20, 200, and 1000 μL.

The steps for preparing the Sample Stock Solutions were as follows: A 1× TE buffer was prepared by combining 10 mL of 20× TE buffer with 190 mL of RNase-free water in a clear glass bottle, which was then shaken to ensure thorough mixing. To 100 mL of the prepared 1× TE buffer, 2 mL of TRITON™ X-100 was added. The solution was stirred using a magnetic stirrer for 15 minutes, resulting in the triton buffer. The 1× TE buffer and triton buffer were poured into separate pipette basins. In the top row (Row A) of the 96-well plate, 10 μL of RNA-LNP sample was added to wells S1-S11, and 10 μL of Formulation buffer was added to the blank well (B). Using a multi-channel pipette, 1× TE buffer was added to Row A to reach a final volume of 250 μL, and the contents were pipetted to ensure thorough mixing.

The RNA-LNP Sample Setup was executed according to the following steps: 50 μL of 1× TE buffer was added to the two wells directly below each RNA-LNP sample in Rows B and C. The RNA-LNP sample stock solution from Row A (50 μL) was transferred into the wells in Rows B and C. 50 μL of triton buffer was added to the wells in Rows D and E, below each sample. The sample stock solution from Row A (50 μL) was added into the wells in Rows D and E. The RNA Standard Curve was set up according to the provided table in duplicate rows F and G, using the RNA Stock (20 μg/mL RNA), 1× TE Buffer, and triton buffer as outlined:

TABLE 12

| Final | RNA Stock | TE Buffer | Triton Buffer | Total Volume |
|---|---|---|---|---|
| RNA | Required | Required | Required | per Well |
| μg/mL | (μL) | (μL) | (μL) | (μL) |
| 2.5 | 25 | 25 | 50 | 100 |
| 1 | 10 | 40 | 50 | 100 |
| 0.5 | 5 | 45 | 50 | 100 |
| 0.25 | 2.5 | 47.5 | 50 | 100 |
| 0.1 | 1 | 49 | 50 | 100 |
| 0.05 | 5* | 45 | 50 | 100 |
| 0.025 | 2.5* | 47.5 | 50 | 100 |
| 0 | 0 | 50 | 50 | 100 |

*1:10 diluted RNA stock (2 μg/mL RNA)

Once the samples and standard curve were plated, the plate was incubated at 37° C. for minutes to lyse the RNA-LNPs in the presence of triton buffer.

The RIBOGREEN® Solution was prepared by first determining the total number of required wells for samples and standard curves, adding 4 to this count, and then multiplying the sum by 100 to ascertain the necessary volume. This total volume guided the dilution of RIBOGREEN® Reagent at a 1:100 ratio with 1× TE buffer in a 15 mL RNase-Free Falcon Tube. For instance, if 3000 μL of RIBOGREEN® Solution was calculated, 30 μL of RIBOGREEN® Reagent was mixed with 2970 μL of 1× TE buffer. Following this, the solution underwent 10 seconds of vortexing to ensure thorough mixing before use.

The 96-well plate was removed from the 37° C. incubator, and 100 μL of RIBOGREEN® Solution was added to each well. Any bubbles present were carefully removed using a needle. Subsequently, readings were taken using a fluorescent plate reader, configuring the settings for Excitation at 480 nm, Emission at 520 nm, and Top Read optics. The Gain and Read height were adjusted according to the instrument's specifications to obtain OD Values.

The analysis of the samples involved determining the concentrations of total mRNA (from lysed LNP) and unencapsulated mRNA (from non-lysed LNP) using the standard curve established earlier. Additionally, encapsulation efficiency was calculated using the formula: % EE=[1−(OD of Non-Lysed LNP/OD of Lysed LNP)]×100. These calculations were carried out to evaluate the encapsulation effectiveness of the mRNA within the lipid nanoparticles.

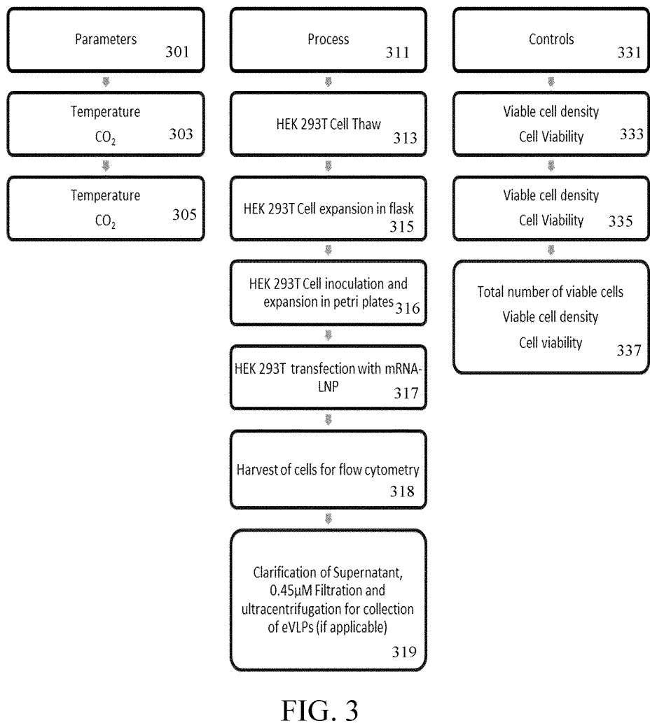

Transfection Assay. FIG. 2 provides a summarized flow diagram depicting the mRNA-LNP transfection process. FIG. 3 presents a process flow diagram detailing the cell culture procedures.

Briefly, in FIG. 2, vials of HEK 293T Master Cell Bank (201) were thawed and cultured/recovered in complete media, comprising High glucose DMEM media supplemented with 10% Fetal bovine serum and 1% Penicillin streptomycin solution (203). These cells were incubated stationary for 48 hours in T75/T182-flasks at 37° C. with a 5% CO₂ environment. Throughout this period, the cells were allowed to grow and expand until they reached confluency (>80%) (205). Upon achieving the desired confluency, the cells were sub-cultured into 10 sq. cm petri-plates using complete media at a cell density ranging from 1.8 to 2.0 million cells per milliliter.

On the day following cell seeding, the media was gently removed from the plates. mRNA-LNP was diluted in opti-MEM medium at a ratio ranging from 1:3 to 1:5 and added to the plates. Additionally, 9 mL of complete medium was introduced before returning the cells to the incubator for a duration of 24 to 48 hours at 37° C. with a 5% CO₂ atmosphere. See cell transfection step (207).

After 24-48 hours post-transfection, the transfected cells were harvested (209) following a specific procedure: Initially, the supernatant was extracted into a 15-50 mL falcon tube and processed separately if needed. A 50 mL falcon tube was labeled accordingly as per the test article. Subsequently, 1 mL of Trypsin-EDTA was added to each 10 mL petri dish, left for 3-5 minutes at room temperature, and gentle rocking dislodged the cells. Then, 10 mL of cold DPBS was added, gently aspirated, and collected into the labeled falcon tube. An additional 10 mL of cold DPBS was added to the plate to gather any remaining cells, and the volume was adjusted to 40 mL with DPBS. The falcon tubes were centrifuged at 1500 RPM for 6 minutes at 2-8° C., after which the supernatant was discarded, and cells were resuspended in approximately 40 mL of cold DPBS. The cells underwent two more washes with subsequent centrifugation steps, totaling three washes, before discarding the supernatant and adding 10 mL of cold DPBS for final resuspension. The cell suspension was passed through a cell strainer into a clean tube to eliminate clumps, cell counts were determined, and a volume equivalent to approximately 0.5 million cells was transferred to another tube. Finally, the tubes were centrifuged at 1500 RPM for 6 minutes at 2-8° C., and the supernatant was decanted.

The surface staining process was conducted as follows: Initially, the DCS (Dead Cell Stain) was diluted in DPBS. Cells, excluding the NO DEAD CELL STAIN control tubes, were resuspended in 150 μL of DCS in DPBS and incubated at 37° C. for 7 minutes. Following incubation, centrifugation at 1500 rpm for 6 min at 2-8° C. was performed, followed by washing with 150 μL of cold stain buffer and another centrifugation step. Subsequently, cells were washed again, centrifuged, and the supernatant was decanted. Cells were then resuspended in 50 μL of Fc block solution in stain buffer. After pipetting and incubating for 10 minutes at room temperature, the cells in tubes were treated with master mixes of stains and Full Minus One (FMO) tubes prepared. The sample master mix, consisting of labeled antibodies diluted in stain buffer, was added to sample tubes/wells, while unstained wells received 50 μL of stain buffer. Pipetting and incubation at 2-8° C. for 30 minutes followed, with subsequent washing using 150 μL of stain buffer before transfer to FACS tubes for reading. A minimum of 300,000 events per tube was acquired using the Celesta flow cytometer, and the data were exported as FCS files for further analysis in FlowJo.

The eVLP collection process involved several steps: Initially, the supernatant (SN) was centrifuged at 1500 rpm for 6 minutes to eliminate cell debris and larger particles. The resulting clarified medium underwent further processing via filtration using a 0.45 μm PVDF filter. Subsequently, the filtered medium was subjected to ultracentrifugation at 16,000 rpm for 2 hours at 2-8° C. to pellet the eVLPs. Upon completion of centrifugation, the supernatant was decanted, leaving intact pellets settled at the tube bottom. These pellets were then resuspended using a cold 10 mM phosphate/8% sucrose buffer. Finally, the resuspended pellets were aliquoted and stored at temperatures between −80° C. to −20° C. for preservation.

FIG. 3 illustrates a general process flow diagram detailing the cell culture procedures noted herein. Parameters (301), process (311) and controls (331) are determined. Temperature and CO₂ (303) are assessed as HEK 293T cells thaw (313), and viable cell density and cell viability (333) are determined. Further, temperature and CO₂ (305) are assessed as HEK 293T cells expansion occurs in flask (315), and viable cell density and cell viability (335) are assessed. HEK 293T cell inoculation and expansion in petri plates occurs (316). A total number of viable cells is determined, together with viable cell density and cell viability (337). HEK 293T cells are transfected with mRNA-LNP (317), and cells are harvested for flow cytometry (318). A clarification of supernatant, with 0.45 μM filtration and ultracentrifugation for collection of eVLPs, if applicable (319).

A short-term stability study spanning 3 months was conducted to explore the stability of an exemplary formulation according to the present disclosure (TA1 of 28CH42A, the “VBI formulation”) under varied storage conditions: 2-8° C., −20° C.±5° C., −80° C.±20° C., and for 1 month at 25° C.±2° C. (Table 13). In parallel, an in-house formulation mimicking an approved LNP composition (TA2 of 28CH42A) was prepared using identical parameters to the VBI formulation and subjected to the same stability assessment.

The investigation focused on several physicochemical parameters—such as particle size, Polydispersity Index (PDI), and zeta potential—to evaluate the stability of the particles. Additionally, to confirm the mRNA content, RIBOGREEN® assay was executed (Table 14).

Upon concluding the 3-month stability study, an evaluation of in vitro potency (bioactivity) was conducted on samples stored under various conditions (Table 15). Specifically, a transfection-based assay involving SARS COV1 mRNA (SEQ ID NO: 33) encapsulated in LNP was undertaken to compare the expression levels and infer any variations or impacts on bioactivity across different storage environments.

The VBI formulations displayed acceptable mRNA recoveries within specified ranges throughout the 3-month storage periods at 2-8° C., −20° C.±5° C., −80° C.±20° C., and for 1 month at 25° C.±2° C., as assessed via the RIBOGREEN® assay. Over this duration, no significant alterations were observed in particle size, Polydispersity Index (PDI), or Zeta Potential (ZP) for the VBI formulations stored under these varied temperature conditions.

Furthermore, a comparative evaluation using a transfection-based in vitro potency assay highlighted consistent performance at −80° C. and −20° C. storage conditions. However, a notable observation emerged for the VBI formulation stored at 2-8° C. for 3 months, exhibiting around 50% bioactivity. Concurrently, the comparative formulation also experienced a significant (approximately 99%) loss of bioactivity under identical storage conditions. Without wishing to be bound by theory, the authors of the present disclosure hypothesize that this decrease in bioactivity might be attributed to potential alterations in mRNA configuration due to accelerated temperature conditions, leading to a loss of functional activity in the formulations.

TABLE 13
SHORT-TERM STABILITY STUDY (28CH42A)

| | | | | | SARS | | |
|---|---|---|---|---|---|---|---|
| | | | | | CoV-1 | | |
| | | | | | spike GP | | |
| Test | Storage | Lipid | Molar Lipid | | mRNA | mRNA | Buffer |
| Articles | Conditions | composition | Ratio (%) | Lipid | (μg/mL) | solution | Composition |
| 1 | −80° C. ± 20° C. | C12-200:DOPE:Cholesterol:PEG2000- | 33.5:19:45.1:2.4 | 4 | 100 | 50 mM of | 10 mM |
| | −20° C.± 5° C. | DMG | | mg/mL | | citrate | Phosphate 8% |
| | 2-8° C. | | | | | buffer | Sucrose, pH 7.2 |
| | 25° C. ± 2° C. | | | | | | |
| 2 | −80° C. ± 20° C. | SM-102:DSPC:Cholesterol:PEG2000- | 50:10:38.5:1.5 | 4 | 100 | 50 mM of | TRIS with |
| | −20° C. ± 5° C. | DMG | | mg/mL | | citrate | 8.7% Sucrose, |
| | 2-8° C. | | | | | buffer | pH 7.2 |
| | 25° C. ± 2° C. | | | | | | |

TABLE 14

| | | −80° C. [symbol] 20° C. | | | | −20° C. [symbol] 5° C. | | | |
|---|---|---|---|---|---|---|---|---|---|
| | | Particle | | | | Particle | | | |
| | Time | Size | | ZP | | Size | | ZP | |
| Test Articles Description | point | (nm) | PDI | (m [symbol] ) | EE % | (nm) | PDI | (m [symbol] ) | EE % |
| C12- | Day 1 | 113.8 | 0.144 | 9.96 | 83.2 | NA | NA | NA | NA |
| 200:DOPE:Cholesterol:PEG2000- | Day 2 | 11 [symbol] .8 | 0.136 | 9.21 | 84.3 | NA | NA | NA | NA |
| DMG (33.5:19:45.1:2.4) | Day 15 | 120 | 0.143 | 9.26 | 82.1 | 135.2 | 0.132 | 9.84 | [symbol] 8.3 |
| with 10 mM Phosphate with | Day 30 | 129.4 | 0.15 [symbol] | 8.21 | 81.1 | 13 [symbol] .3 | 0.148 | 9.38 | [symbol] 8.4 |
| 8% Sucrose, pH [symbol] .2 | Day 60 | 131.6 | 0.168 | 8.6 [symbol] | [symbol] 9. [symbol] | 15 [symbol] .1 | 0.1 [symbol] 8 | 9.21 | 69.9 |
| | Day 90 | 133.8 | 0.13 [symbol] | 9.19 | 80.2 | 159.8 | 0.1 [symbol] 6 | 9.5 [symbol] | [symbol] 2 |
| SM- | Day 1 | 119.6 | 0.189 | 6. [symbol] 3 | 86.6 | NA | NA | NA | NA |
| 102:DSPC:Cholesterol:PEG2000- | Day 2 | 120.8 | 0.18 | 9.32 | 86. [symbol] | NA | NA | NA | NA |
| DMG (50:10:38.5:1.5) | Day 15 | 116.8 | 0.168 | [symbol] .33 | 90.3 | 118. [symbol] | 0.152 | 6. [symbol] 2 | 86.5 |
| with TRIS with 8 [symbol] % | Day 30 | 11 [symbol] .5 | 0.15 [symbol] | 6. [symbol] 5 | 88. [symbol] | 119.1 | 0.15 [symbol] | 5.83 | 86.6 |
| Sucrose, pH [symbol] .2 | Day 60 | 119.1 | 0.156 | 6.3 | [symbol] .6 | 121.5 | 0.153 | 5.95 | [symbol] 5.4 |
| | Day 90 | 119.3 | 0.149 | 9.31 | 8 [symbol] | 121. [symbol] | 0.145 | 6.94 | [symbol] 9. [symbol] |
| | | 2-8° C. | | | | 25° C. [symbol] 2° C. | | | |
| | | Particle | | | | Particle | | | |
| | Time | Size | | ZP | | Size | | ZP | |
| Test Articles Description | point | (nm) | PDI | (m [symbol] ) | EE % | (nm) | PDI | (m [symbol] ) | EE % |
| C12- | Day 1 | 115.4 | 0.132 | 10.3 | 83.3 | 115.8 | 0.156 | 9. [symbol] 6 | [symbol] 8.6 |
| 200:DOPE:Cholesterol:PEG2000- | Day 2 | 142.4 | 0.251 | 10.3 | 83.1 | 134.4 | 0.236 | 11.2 | 85.9 |
| DMG (33.5:19:45.1:2.4) | Day 15 | 129.5 | 0.18 | 8.89 | 82.8 | 120. [symbol] | 0.1 [symbol] 4 | 8.89 | 84.9 |
| with 10 mM Phosphate with | Day 30 | 139.4 | 0.222 | 8.69 | [symbol] 9.9 | 126 | 0.26 | 5.99 | 82.6 |
| 8% Sucrose, pH [symbol] .2 | Day 60 | 155.6 | 0.308 | [symbol] .52 | [symbol] . [symbol] | NA | NA | NA | NA |
| | Day 90 | 162.3 | 0.316 | 8.21 | 80 | NA | NA | NA | NA |
| SM- | Day 1 | 116. [symbol] | 0.152 | 6.9 | 93.2 | 116. [symbol] | 0.1 [symbol] 6 | 6. [symbol] 6 | 91.1 |
| 102:DSPC:Cholesterol:PEG2000- | Day 2 | 119.1 | 0.1 [symbol] | 8.44 | 86.8 | 11 [symbol] .3 | 0.151 | 6.43 | 85 |
| DMG (50:10:38.5:1.5) | Day 15 | 116. [symbol] | 0.166 | [symbol] .25 | 92.8 | 115. [symbol] | 0.131 | 3.63 | 8 [symbol] |
| with TRIS with 8 [symbol] % | Day 30 | 116.6 | 0.164 | 5.84 | 91.9 | 118.5 | 0.133 | 5.82 | 83.6 |
| Sucrose, pH [symbol] .2 | Day 60 | 116.3 | 0.152 | 5.01 | 82.4 | NA | NA | NA | NA |
| | Day 90 | 114.3 | 0.15 [symbol] | [symbol] .05 | 93.5 | NA | NA | NA | NA |

[symbol]

indicates data missing or illegible when filed

TABLE 15

| | | −80° C. [symbol] 20° C. | | −20° C. [symbol] 5° C. | | 2-8° C. | |
|---|---|---|---|---|---|---|---|
| | | Median | % SARS-CoV-1 | Median | % SARS-CoV-1 | Median | SARS-CoV-1 |
| | Time | Fluorescence | positive | Fluorescence | positive | Fluorescence | positive |
| Test Articles Description | point | Intensity | Cells | Intensity | Cells | Intensity | Cells |
| C12-200:DOPE:Cholesterol:PEG2000- | Day 90 | 16445 | 99.10% | 99 [symbol] 9 | 9 [symbol] .80% | 1 [symbol] 69 | 46.10% |
| DMG (33.5:19:45.1:2.4) | | | | | | | |
| with 10 mM Phosphate with | | | | | | | |
| 8% Sucrose, pH [symbol] .2 | | | | | | | |
| SM-102:DSPC:Cholesterol:PEG2000- | | 8569 | 95. [symbol] 0% | [symbol] 893 | 96.00% | 5 [symbol] 0 | 1.58% |
| DMG (50:10:38.5:1.5) | | | | | | | |
| with TRIS with 8 [symbol] % | | | | | | | |
| Sucrose, pH [symbol] .2 | | | | | | | |

[symbol]

indicates data missing or illegible when filed

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be made to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

### Aspects

Various aspects are described in the paragraphs below.

Aspect 1. An immunogenic composition comprising mRNA encoding polypeptides that constitute a virus-like particle (VLP), and a pharmaceutically acceptable carrier, wherein the polypeptides that constitute the VLP comprise: a first polypeptide comprising a gag protein found in murine leukemia virus (MLV) having at least 95% identity with the amino acid sequence of SEQ ID NO:1; and at least one additional polypeptide comprising a protein of a virus against which an immunogenic effect is directed.

Aspect 2. The immunogenic composition of Aspect 1, wherein the at least one additional polypeptide comprises a protein of Epstein-Barr virus (EBV) or cytomegalovirus (CMV).

Aspect 3. The immunogenic composition of Aspect 1, wherein the at least one additional polypeptide comprises a spike glycoprotein from a beta coronavirus.

Aspect 4. The immunogenic composition of Aspect 3, wherein the at least one additional polypeptide comprises a spike glycoprotein from SARS-COV-2, SARS-COV-1 or MERS-COV.

Aspect 5. The immunogenic composition of Aspect 4, wherein the spike glycoprotein is a wild type protein.

Aspect 6. The immunogenic composition of Aspect 4, wherein the spike glycoprotein is a modified protein.

Aspect 7. The immunogenic composition of any one of Aspect s 3 to 6, wherein the at least one additional polypeptide comprises two spike glycoproteins.

Aspect 8. The immunogenic composition of any one of Aspect s 3 to 6, wherein the at least one additional polypeptide comprises three spike glycoproteins.

Aspect 9. The immunogenic composition of claim 6, wherein the modified protein comprises: a deletion at a furin cleavage site, or one or more lysine and/or valine residues is replaced with a proline residue.

Aspect 10. The immunogenic composition of any one of Aspects 1 to 9, further comprising an adjuvant.

Aspect 11. The immunogenic composition of Aspect 10, wherein the adjuvant is selected from the group consisting of cytokines, gel-type adjuvants, microbial adjuvants, oil-emulsion and emulsifier-based adjuvants, particulate adjuvants, synthetic adjuvants, polymer adjuvants, and combinations thereof.

Aspect 12. The immunogenic composition of Aspect 11, wherein the particulate adjuvant is an aluminum salt.

Aspect 13. The immunogenic composition of any one of Aspect s 1 to 12, wherein the composition is formulated as lipid nanoparticles (LNPs) loaded with the mRNA.

Aspect 14. The immunogenic composition of any one of Aspect s 1 to 13, comprising two or more mRNAs encoding the polypeptides constituting the VLP.

Aspect 15. The immunogenic composition of any one of Aspect s 1 to 13, wherein one mRNA encodes the two or more of the polypeptides constituting the VLP.

Aspect 16. An immunogenic composition comprising mRNA encoding polypeptides that constitute a virus-like particle, and a pharmaceutically acceptable carrier, wherein the polypeptides that constitute the VLP comprise: a first polypeptide comprising a gag protein found in murine leukemia virus having at least 95% identity with the amino acid sequence of SEQ ID NO:1; and at least one additional polypeptide comprising a protein expressed by a tumor cell or cancer cell against which an immunogenic effect is directed.

Aspect 17. A method of treatment or prophylaxis of a subject having or at risk for a viral infection, comprising administering to the subject the immunogenic composition of Aspect 1, wherein the at least one additional polypeptide comprises a protein expressed by a virus or a virally infected cell against which the immunogenic effect of the composition is directed.

Aspect 17A. Use of the immunogenic composition of Aspect 1 for treatment or prophylaxis, or for preparation of a medicament for treatment or prophylaxis, of a subject having or at risk for a viral infection, wherein the at least one additional polypeptide comprises a protein expressed by a virus or a virally infected cell against which the immunogenic effect of the composition is directed.

Aspect 17B. The immunogenic composition of Aspect 1 for use in a method of treatment or prophylaxis of a subject having or at risk for a viral infection, wherein the at least one additional polypeptide comprises a protein expressed by a virus or a virally infected cell against which the immunogenic effect of the composition is directed.

Aspect 18. A method of treatment or prophylaxis of a subject having or at risk for Epstein-Barr virus (EBV) or cytomegalovirus (CMV) infection, comprising administering to the subject the immunogenic composition of Aspect 2.

Aspect 18A. Use of the immunogenic composition of Aspect 2 for treatment or prophylaxis, or for preparation of a medicament for treatment or prophylaxis, of a subject having or at risk for Epstein-Barr virus (EBV) or cytomegalovirus (CMV) infection.

Aspect 18B. The immunogenic composition of Aspect 2 for use in a method of treatment or prophylaxis of a subject having or at risk for Epstein-Barr virus (EBV) or cytomegalovirus (CMV) infection.

Aspect 19. A method of treatment or prophylaxis of a subject having or at risk for coronavirus infection, comprising administering to the subject the immunogenic composition of any one of Aspects 3 to 9.

Aspect 19A. Use of the immunogenic composition of any one of Aspects 3 to 9 for treatment or prophylaxis, or for preparation of a medicament for treatment or prophylaxis, of a subject having or at risk for coronavirus infection.

Aspect 19B. The immunogenic composition of any one of Aspects 3 to 9 for use in a method of treatment or prophylaxis of a subject having or at risk for coronavirus infection.

Aspect 20. A method of treatment or prophylaxis of a subject against a tumor or a cancer, comprising administering to the subject the immunogenic composition of Aspect 16, wherein the at least one additional polypeptide comprises a protein expressed by the tumor cell or cancer cell against which the immunogenic effect is directed.

Aspect 20A. Use of the immunogenic composition of Aspect 16 for treatment or prophylaxis, or for preparation of a medicament for treatment or prophylaxis, of a subject against a tumor or a cancer, wherein the at least one additional polypeptide comprises a protein expressed by the tumor cell or cancer cell against which the immunogenic effect is directed.

Aspect 20B. The immunogenic composition of Aspect 16 for use in a method of treatment or prophylaxis of a subject against a tumor or a cancer, wherein the at least one additional polypeptide comprises a protein expressed by the tumor cell or cancer cell against which the immunogenic effect is directed.

Aspect 21. A method of producing a virus-like particle (VLP) by co-transfecting a host cell with a first vector comprising a nucleotide sequence encoding SEQ ID NO: 1 and a second vector comprising a nucleotide sequence of one or more of SEQ ID NOs: 6, 7, 10, 11, 14, 15, 18, 19, 22, 23, 27 or 31; and cultivating the host cell under conditions suitable for expression of the proteins encoded by the vectors.

Aspect 22. A lipid nanoparticle composition comprising C12-200, DOPE, cholesterol, and PEG-2000 DMG in a molar ratio of 33.5±1.7:19=1:45.1±2.3:2.4±0.1, optionally wherein the lipids are present at a concentration of about 2 to about 6 mg/mL, for example about 3 mg/mL to about 5 mg/mL, such as about 4 mg/mL.

Aspect 23. The lipid nanoparticle composition according to Aspect 22, further comprising: phosphate at a concentration of about 8 mM to about 12 mM, such as about 10 mM; and sucrose, trehalose, or a combination thereof at a concentration of about 7 wt % to about 9 wt %, such as about 8 wt %; wherein the composition is at a pH of about 7.0 to about 7.4, such as a pH of about 7.2.

Aspect 24. The lipid nanoparticle composition according to Aspect 22 or 23, further comprising the immunogenic composition as defined in any one of Aspect 1 to 16.

Aspect 25. The lipid nanoparticle composition according to Aspect 22 or 23, further comprising mRNA, wherein the mRNA encodes polypeptides that constitute a virus-like particle (VLP), and the polypeptides that constitute the VLP comprise: a first polypeptide comprising a gag protein found in murine leukemia virus (MLV) having at least 95% identity with the amino acid sequence of SEQ ID NO:1; and at least one additional polypeptide comprising a protein of a virus against which an immunogenic effect is directed.

Aspect 26. The lipid nanoparticle of Aspect 25, wherein the at least one additional polypeptide comprises a protein of Epstein-Barr virus (EBV) or cytomegalovirus (CMV).

Aspect 27. The lipid nanoparticle composition of Aspect 25, wherein the at least one additional polypeptide comprises a spike glycoprotein from a beta coronavirus.

Aspect 28. The lipid nanoparticle of claim 27, wherein the at least one additional polypeptide comprising a spike protein from a beta coronavirus comprises the additional at least one polypeptide as defined in any one of Aspect s 4 to 9.

Aspect 29. The lipid nanoparticle composition according to claim 22 or 23, further comprising mRNA, wherein the mRNA encodes polypeptides that constitute a virus-like particle (VLP), and the polypeptides that constitute the VLP comprise:
a first polypeptide comprising a gag protein found in murine leukemia virus (MLV) having at least 95% identity with the amino acid sequence of SEQ ID NO:1; and
at least one additional polypeptide comprising a protein expressed by a tumor cell or cancer cell against which an immunogenic effect is directed.

Aspect 30. A method of treatment or prophylaxis of a subject having or at risk for a viral infection, comprising administering to the lipid nanoparticle composition of Aspect 25.

Aspect 30A. Use of the lipid nanoparticle composition of Aspect 25 for treatment or prophylaxis, or for preparation of a medicament for treatment or prophylaxis, of a subject having or at risk for a viral infection.

Aspect 30B. The lipid nanoparticle composition of Aspect 25 for use in a method of treatment or prophylaxis of a subject having or at risk for a viral infection.

Aspect 31. A method of treatment or prophylaxis of a subject having or at risk for Epstein-Barr virus (EBV) or cytomegalovirus (CMV) infection, comprising administering to the subject the lipid nanoparticle composition of Aspect 26.

Aspect 31A. Use of the lipid nanoparticle composition of Aspect 26 for treatment or prophylaxis, or for preparation of a medicament for treatment or prophylaxis, of a subject having or at risk for Epstein-Barr virus (EBV) or cytomegalovirus (CMV) infection.

Aspect 31B. The lipid nanoparticle composition of Aspect 26 for use in a method of treatment or prophylaxis of a subject having or at risk for Epstein-Barr virus (EBV) or cytomegalovirus (CMV) infection.

Aspect 32. A method of treatment or prophylaxis of a subject having or at risk for coronavirus infection, comprising administering to the subject the lipid nanoparticle composition of Aspect 27 or 28.

Aspect 32A. Use of the lipid nanoparticle composition of Aspect 27 or 28 for treatment or prophylaxis, or for preparation of a medicament for treatment or prophylaxis, of a subject having or at risk for coronavirus infection.

Aspect 32B. The lipid nanoparticle composition of Aspect 27 or 28 in a method of treatment or prophylaxis of a subject having or at risk for coronavirus infection.

Aspect 33. A method of treatment or prophylaxis of a subject against a tumor or a cancer, comprising administering to the subject the lipid nanoparticle composition of Aspect 29.

Aspect 33A. Use of the lipid nanoparticle composition of Aspect 29 for treatment or prophylaxis, or for preparation of a medicament for treatment or prophylaxis, of a subject against a tumor or a cancer.

Aspect 33B. The lipid nanoparticle composition of Aspect 29 for use in a method of treatment or prophylaxis of a subject against a tumor or a cancer.

### REFERENCES

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.
Chaudhary N et al. (2021) Nat Rev Drug Discov. (11): 817-838. PMID: 34433919
Compton T et al., (1989) Proc Natl Acad Sci USA 86:4112-4116
Cui et al., (2021) Frontiers in Immunology 12, Article 734471
Garrone P et al., (2011) Sci Transl Med 3 (94): 94ra71
Gilbert et al., (2022) N Engl J Med 387:2203-2206
Han et al., (2021) Nature Communications 12:7233
Hoffmann M A G et al., (2023) Cell 186 (11) 2283-2285
Lu J et al., (2020) Cell Research (30) p. 936-939
McCarthy et al., (2014) Journal of Controlled Release (189) p. 141-149
Nooraei S et al., (2021) Journal of Nanobiotechnology (19), Article no: 59
Panagioti E et al., (2018) Front. Immunol., Sec. T Cell Biology (Vol 9) Article no: 2509
Plotkin S A et al., (2010) Clin Vaccine Immunol. 17 (7): 1055-1065
Ramesh P et al., (2021) Cancers (13), p.821
Sette and Crotty, (2022) Immunol Rev 310 (1): 27-46
Sharma S et al., (1997) Proc. Natl. Acad. Sci. USA 94:10803-8
Tellam et al., (2001) JBC 276 (36), pp. 33353-33360
Yan D et al., (2015) Applied Microbiology and Biotechnology (99), p. 10415-10432

1. An immunogenic composition comprising mRNA encoding polypeptides that constitute a virus-like particle (VLP), and a pharmaceutically acceptable carrier,
wherein the polypeptides that constitute the VLP comprise:
a first polypeptide comprising a gag protein found in murine leukemia virus (MLV) having at least 95% identity with the amino acid sequence of SEQ ID NO: 1; and
at least one additional polypeptide comprising:
a protein of a virus against which an immunogenic effect is directed; or
a protein expressed by a tumor cell or cancer cell against which an immunogenic effect is directed.

2. The immunogenic composition of claim 1, wherein the at least one additional polypeptide comprises a protein of Epstein-Barr virus (EBV) or cytomegalovirus (CMV).

3. The immunogenic composition of claim 1, wherein the at least one additional polypeptide comprises a spike glycoprotein from a beta coronavirus, SARS-COV-2, SARS-COV-1 or MERS-COV.

4. (canceled)

5. The immunogenic composition of claim 3, wherein the spike glycoprotein is a wild type protein or is a modified protein.

6. (canceled)

7. The immunogenic composition of claim 3, wherein the at least one additional polypeptide comprises two or three spike glycoproteins.

8. (canceled)

9. The immunogenic composition of claim 5, wherein the modified protein comprises:
a deletion at a furin cleavage site, or
one or more lysine and/or valine residues is replaced with a proline residue.

10. The immunogenic composition of further comprising an adjuvant selected from the group consisting of cytokines, gel-type adjuvants, microbial adjuvants, oil-emulsion and emulsifier-based adjuvants, particulate adjuvants, an aluminum salt, synthetic adjuvants, polymer adjuvants, and combinations thereof.

11. (canceled)

12. (canceled)

13. The immunogenic composition of claim 1, wherein the composition is formulated as lipid nanoparticles (LNPs) loaded with the mRNA.

14. The immunogenic composition of claim 1, comprising
comprising two or more mRNAs encoding the polypeptides constituting the VLP; or
wherein one mRNA encodes the two or more of the polypeptides constituting the VLP.

15. (canceled)

16. (canceled)

17. A method of treatment or prophylaxis of a subject having or at risk for a viral infection, comprising administering to the subject the immunogenic composition of claim 1, wherein the at least one additional polypeptide comprises a protein expressed by a virus or a virally infected cell against which the immunogenic effect of the composition is directed.

18. A method of treatment or prophylaxis of a subject having or at risk for Epstein-Barr virus (EBV) or cytomegalovirus (CMV) infection, comprising administering to the subject the immunogenic composition of claim 2.

19. A method of treatment or prophylaxis of a subject having or at risk for coronavirus infection, comprising administering to the subject the immunogenic composition of claim 3.

20. A method of treatment or prophylaxis of a subject against a tumor or a cancer, comprising administering to the subject the immunogenic composition of claim 1, wherein the at least one additional polypeptide comprises a protein expressed by the tumor cell or cancer cell against which the immunogenic effect is directed.

21. A method of producing a virus-like particle (VLP) by co-transfecting a host cell with a first vector comprising a nucleotide sequence encoding SEQ ID NO: 1 and a second vector comprising a nucleotide sequence of one or more of SEQ ID NOs: 6, 7, 10, 11, 14, 15, 18, 19, 22, 23, 27 or 31; and
cultivating the host cell under conditions suitable for expression of the proteins encoded by the vectors.

22. A lipid nanoparticle composition comprising:
C12-200, DOPE, cholesterol, and PEG-2000 DMG in a molar ratio of 33.5±1.7:19±1:45.1±2.3:2.4±0.1, optionally wherein the lipids are present at a concentration of about 2 to about 6 mg/mL, about 3 mg/mL to about 5 mg/mL, or about 4 mg/ml; and
the immunogenic composition of claim 1.

23. The lipid nanoparticle composition according to claim 22, further comprising:
phosphate at a concentration of about 8 mM to about 12 mM, such as about 10 mM; and
sucrose, trehalose, or a combination thereof at a concentration of about 7 wt % to about 9 wt %, such as about 8 wt %;
wherein the composition is at a pH of about 7.0 to about 7.4, such as a pH of about 7.2.

24. (canceled)

25. (canceled)

26. The lipid nanoparticle composition of claim 22, wherein the at least one additional polypeptide comprises a protein of Epstein-Barr virus (EBV) or cytomegalovirus (CMV).

27. The lipid nanoparticle composition of claim 22, wherein the at least one additional polypeptide comprises a spike glycoprotein from a beta coronavirus.

28. (canceled)

29. The lipid nanoparticle composition according to claim 22, wherein the
at least one additional polypeptide comprises a protein expressed by a tumor cell or cancer cell against which an immunogenic effect is directed.

30. A method of treatment or prophylaxis of a subject having or at risk for:
a viral infection, Epstein-Barr virus (EBV) or cytomegalovirus (CMV) infection, coronavirus infection, a tumor or a cancer, comprising administering to the subject the lipid nanoparticle composition of claim 22.

31. (canceled)

32. (canceled)

33. (canceled)


SEQUENCE LISTING (34 sequences)

SEQ ID NO: 1 - protein, 538 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 2 - DNA, 1617 bp, synthetic construct
  source 1..1617: mol_type = other DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactaa

SEQ ID NO: 3 - DNA, 1617 bp, synthetic construct
  source 1..1617: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgactaa

SEQ ID NO: 4 - RNA, 1617 nt, synthetic construct
  source 1..1617: mol_type = other RNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgactaa

SEQ ID NO: 5 - protein, 913 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDMSDEGPGTGPGNGLGQKEDTSGPEGSGGSGPQRRGGDNHGRGRGRGRGRGGGRPGAPGGSGSGPRHRDGVRRPQKRPSCIGCKGAHRRGRGRERARGGSRERARGRGRGRGEKRPRSPSSQSSSSGSPPRRPPPGRRPFFHPVGEADYFEYHQEGGPDGEPDVPPGAIEQGPADDPGEGPSTGPRGQGDGGRRKKGGWFGKHRGQGGSNPKFENIAEGLRALLARSHVERTTDEGTWVAGVFVYGGSKTSLYNLRRGIALAIPQCRLTPLSRLPFGMAPGPGPQPGPLRESIVCYFMVFLQTHIFAEVLKDAIKDLVMTKPAPTCNIKVTVCSFDDGVDLPPWFPPMVEGAAAEGDDGDDGDEGGDGDEGEEGQE

SEQ ID NO: 6 - DNA, 2742 bp, synthetic construct
  source 1..2742: mol_type = other DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgacatgtctgacgaggggccaggtacaggacctggaaatggcctaggacagaaggaggacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaaggggcccaccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaattgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcaaagtgactgtgtgcagctttgacgatggagtagatttgcctccctggtttccacctatggtggaaggggctgccgcggagggtgatgacggagatgacggagatgaaggaggtgatggagatgagggtgaggaagggcaggagtga

SEQ ID NO: 7 - DNA, 2742 bp, synthetic construct
  source 1..2742: mol_type = other DNA
atggggcagacagtgacaactcctctgagcctgaccctggggcattggaaagacgtggaacgaatcgcacataaccagtccgtggatgtgaagaagcgcaggtgggtgaccttctgttctgccgagtggccaaccttcaacgtgggctggccccgggatggcacctttaatcgggatctgatcacacaggtgaagatcaaggtgttttctccaggcccacacggccaccctgatcaggtgccctatatcgtgacatgggaggccctggccttcgacccccccccatgggtgaagcccttcgtgcacccaaagccccccccccccctgcccccatccgccccctccctgcccctggagccacccaggtccaccccccccagaagcagcctgtaccctgccctgaccccatccctgggcgcgaagcccaagccacaggtgctgagcgattctggcggcccactgatcgacctgctgacagaggacccacccccatatcgggaccctcgccctcctccatctgatagagacggcaatggcggcgaggccacacccgccggcgaggcccccgatcctagccccatggcctcccgcctgaggggcagaagggagccacccgtggccgacagcacaacctctcaggcctttcctctgcgggccggcggcaacggccagctacagtattggcccttcagcagctctgatctgtacaactggaagaacaataacccttccttttctgaggaccccggcaagctgaccgccctgatcgagtctgtgctgatcacccaccagcccacctgggatgactgtcagcagctactgggcacactgctgacaggcgaggagaagcagcgggtgctgctggaggccagaaaggccgtgcggggcgatgacggcaggccaacacagctacccaacgaggtggatgccgcctttcctctggagaggccagactgggactatacaacccaggccgggcgcaaccacctggtgcactaccggcagctactgctggccggcctgcagaacgccggcaggtcccccacaaatctggccaaggtgaagggcatcacacagggccctaacgagtctccctctgccttcctggagagactgaaggaggcctaccggagatacacaccctatgatccagaggacccaggccaggagacaaatgtatccatgtccttcatctggcagtccgccccagacatcggcagaaagctggagcggctggaggacctgaagaacaagaccctgggcgacctggtgcgggaggccgagaagatcttcaataagcgcgagacccctgaggagcgggaggagagaatcagacgcgagaccgaggagaaggaggagaggaggaggaccgaggacgagcagaaggagaaggagagagatagaaggcggcaccgggagatgtccaagctgctggccaccgtggtgtccggccagaagcaggaccggcagggcggcgagcgccggagatctcagctagaccgcgatcagtgcgcctactgtaaggagaagggccactgggccaaggactgccccaagaagccaaggggcccccggggcccaaggcctcagacctccctgctgaccctggacgacatgtccgatgagggccccggcacaggcccaggcaacggcctgggccagaaggaggacacctccggccctgagggctccggcggcagcggccctcagaggaggggcggcgacaatcacggcagaggccggggccggggccggggcaggggcggcggcagaccaggcgcgcccggcggctccggcagcggccccaggcacagagatggcgtgcgcagaccccagaagcggccttcttgtatcggctgcaagggcgcgcacaggagaggccggggccgggagagggcccggggcggcagcagggagcgcgccagaggccggggcagaggcagaggcgagaagagaccacggtctccctcttcccagtcctcctctagcggctcccccccacgccggcctccccccgggcggagaccatttttccacccagtgggcgaggccgattactttgagtatcaccaggagggcggccctgatggcgagcccgatgtgccacccggcgcgatcgagcagggccccgccgacgatccaggcgagggccccagcaccggccccaggggccagggcgacggcggcagacgcaagaagggcggctggtttggcaagcaccggggccagggcggctccaatccaaagtttgagaatatcgccgagggcctgcgggccctgctggccagatcccacgtagagaggaccaccgacgagggcacctgggtggccggcgtgtttgtatatggcggctctaagacaagcctgtacaacctgcggagaggcatcgccctggccatcccacagtgtagactgacaccactgagcagactgccattcggcatggcccctggcccaggccctcagcccggccctctgagagagtccatcgtgtgctacttcatggtgttcctgcagacccacatctttgccgaggtgctgaaggatgccatcaaggacctggtaatgacaaagcctgcccccacatgcaatatcaaggtgacagtgtgctcctttgacgacggcgtggacctgcccccatggtttcccccaatggtggagggcgcagccgccgagggcgacgatggcgacgatggggacgagggaggcgacggcgacgagggagaagaaggacaggagtaa

SEQ ID NO: 8 - RNA, 2742 nt, synthetic construct
  source 1..2742: mol_type = other RNA
atggggcagacagtgacaactcctctgagcctgaccctggggcattggaaagacgtggaacgaatcgcacataaccagtccgtggatgtgaagaagcgcaggtgggtgaccttctgttctgccgagtggccaaccttcaacgtgggctggccccgggatggcacctttaatcgggatctgatcacacaggtgaagatcaaggtgttttctccaggcccacacggccaccctgatcaggtgccctatatcgtgacatgggaggccctggccttcgacccccccccatgggtgaagcccttcgtgcacccaaagccccccccccccctgcccccatccgccccctccctgcccctggagccacccaggtccaccccccccagaagcagcctgtaccctgccctgaccccatccctgggcgcgaagcccaagccacaggtgctgagcgattctggcggcccactgatcgacctgctgacagaggacccacccccatatcgggaccctcgccctcctccatctgatagagacggcaatggcggcgaggccacacccgccggcgaggcccccgatcctagccccatggcctcccgcctgaggggcagaagggagccacccgtggccgacagcacaacctctcaggcctttcctctgcgggccggcggcaacggccagctacagtattggcccttcagcagctctgatctgtacaactggaagaacaataacccttccttttctgaggaccccggcaagctgaccgccctgatcgagtctgtgctgatcacccaccagcccacctgggatgactgtcagcagctactgggcacactgctgacaggcgaggagaagcagcgggtgctgctggaggccagaaaggccgtgcggggcgatgacggcaggccaacacagctacccaacgaggtggatgccgcctttcctctggagaggccagactgggactatacaacccaggccgggcgcaaccacctggtgcactaccggcagctactgctggccggcctgcagaacgccggcaggtcccccacaaatctggccaaggtgaagggcatcacacagggccctaacgagtctccctctgccttcctggagagactgaaggaggcctaccggagatacacaccctatgatccagaggacccaggccaggagacaaatgtatccatgtccttcatctggcagtccgccccagacatcggcagaaagctggagcggctggaggacctgaagaacaagaccctgggcgacctggtgcgggaggccgagaagatcttcaataagcgcgagacccctgaggagcgggaggagagaatcagacgcgagaccgaggagaaggaggagaggaggaggaccgaggacgagcagaaggagaaggagagagatagaaggcggcaccgggagatgtccaagctgctggccaccgtggtgtccggccagaagcaggaccggcagggcggcgagcgccggagatctcagctagaccgcgatcagtgcgcctactgtaaggagaagggccactgggccaaggactgccccaagaagccaaggggcccccggggcccaaggcctcagacctccctgctgaccctggacgacatgtccgatgagggccccggcacaggcccaggcaacggcctgggccagaaggaggacacctccggccctgagggctccggcggcagcggccctcagaggaggggcggcgacaatcacggcagaggccggggccggggccggggcaggggcggcggcagaccaggcgcgcccggcggctccggcagcggccccaggcacagagatggcgtgcgcagaccccagaagcggccttcttgtatcggctgcaagggcgcgcacaggagaggccggggccgggagagggcccggggcggcagcagggagcgcgccagaggccggggcagaggcagaggcgagaagagaccacggtctccctcttcccagtcctcctctagcggctcccccccacgccggcctccccccgggcggagaccatttttccacccagtgggcgaggccgattactttgagtatcaccaggagggcggccctgatggcgagcccgatgtgccacccggcgcgatcgagcagggccccgccgacgatccaggcgagggccccagcaccggccccaggggccagggcgacggcggcagacgcaagaagggcggctggtttggcaagcaccggggccagggcggctccaatccaaagtttgagaatatcgccgagggcctgcgggccctgctggccagatcccacgtagagaggaccaccgacgagggcacctgggtggccggcgtgtttgtatatggcggctctaagacaagcctgtacaacctgcggagaggcatcgccctggccatcccacagtgtagactgacaccactgagcagactgccattcggcatggcccctggcccaggccctcagcccggccctctgagagagtccatcgtgtgctacttcatggtgttcctgcagacccacatctttgccgaggtgctgaaggatgccatcaaggacctggtaatgacaaagcctgcccccacatgcaatatcaaggtgacagtgtgctcctttgacgacggcgtggacctgcccccatggtttcccccaatggtggagggcgcagccgccgagggcgacgatggcgacgatggggacgagggaggcgacggcgacgagggagaagaaggacaggagtaa

SEQ ID NO: 9 - protein, 1374 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTQMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPRDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSIPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFS

SEQ ID NO: 10 - DNA, 4125 bp, synthetic construct
  source 1..4125: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgacgatcctgggtttttcaatgtggagatccctgagtttccattctacccaacatgcaacgtgtgcaccgccgacgtgaacgtgaccatcaattttgatgtgggcggcaagaagcaccagctagacctggattttggccagctaacacctcacaccaaggccgtgtatcagccacgcggcgcgtttggcggcagcgagaacgccacaaacctgtttctgctggagctgctgggcgcgggcgagctggccctgaccatgagaagcaagaagctgccaatcaacgtgaccaccggcgaggagcagcaggtgtccctggagtccgtggacgtgtacttccaggacgtgttcggcacaatgtggtgccaccacgccgagatgcagaacccagtgtacctgatcccagagaccgtgccatacatcaagtgggacaactgcaactccaccaatatcacagccgtggtgcgggcccagggcctggatgtgacactgcctctgagcctgcctacatctgcccaggacagcaacttttccgtgaagacccagatgctgggcaacgagatcgacatcgagtgcatcatggaggatggcgagatctcccaggtgctgcctggcgacaataagttcaacatcacatgtagcggctacgagagccacgtaccttccggcggcatcctgacctctacaagcccagtggccaccccaatccccggcaccggctatgcctactctctgaggctgacacctcggcccgtgagcagattcctgggcaacaactccatcctgtacgtgttctactccggcaatggccctaaggccagcggcggcgactactgtatccagagcaacatcgtgttttccgacgagatccctgcctctcaggatatgcccaccaataccaccgacatcacatacgtgggcgacaatgccacatatagcgtgccaatggtgaccagcgaggacgccaatagccctaacgtgacagtgaccgccttttgggcctggcccaacaatacagagaccgatttcaagtgtaagtggaccctgaccagcggcaccccttccggctgcgagaatatcagcggcgcgtttgcctctaaccggacattcgacatcacagtgtccggcctgggcacagcccccaagaccctgatcatcaccagaaccgccacaaacgccacaacaacaacccacaaggtaatcttttccaaggccccagagtctaccacaacatcccccaccctgaataccacaggcttcgccgaccctaataccacaaccggcctgccttctagcacccacgtaccaacaaacctgaccgccccagcctccacaggcccaaccgtgtccacagccgacgtgacctcccctacccccgccggcaccacatccggcgcgagcccagtgaccccatccccttcccccagggataatggcacagagagcaaggcccccgatatgacatcctctacaagccctgtgaccacccctacccccaacgccaccagccccaccccagccgtgaccaccccaacacccaacgccacctcccctacacctgccgtgacaaccccaacacctaatgccacatccccaacactgggcaagacctccccaacatccgccgtgacaacaccaacccccaatgccacctctccaacactgggcaagacatcccctacctccgccgtgaccacacctaccccaaacgccacatcccccacactgggcaagaccagcccaacatctgccgtgacaaccccaaccccaaatgccacaggccccaccgtgggcgagacaagccctcaggccaatgccacaaatcacaccctgggcggcacctcccctacacccgtggtgacatctcagcccaagaatgccacatctgccgtgacaacaggccagcacaatatcacatcctctagcacatcctccatgtccctgcgcccctcttccatcccagagacactgagcccaagcacctccgacaactccacatctcacatgccactgctgacctctgcccaccccacaggcggcgagaacatcacccaggtgacacctgcctccatcagcacccaccacgtatctaccagctcccctgccccaagaccaggcacaacaagccaggcctccggcccaggcaattcttccacatctaccaagccaggcgaggtgaacgtgaccaagggcacccccccacagaatgccacctcccctcaggccccttccggccagaagacagccgtgccaaccgtgacatccaccggcggcaaggccaattctacaacaggcggcaagcacaccaccggccacggcgcgagaaccagcaccgagcctacaaccgactacggcggcgattccacaaccccaagacccagatacaacgccaccacctacctgcccccttccacaagctctaagctgagaccaagatggacattcacatcccccccagtgaccacagcccaggccacagtgcccgtgccccccaccagccagccacggttctcctaa

SEQ ID NO: 11 - DNA, 4125 bp, synthetic construct
  source 1..4125: mol_type = other DNA
atgggccagacagtgaccacccctctgtccctgaccctgggccactggaaggacgtggaaagaatcgcccacaaccagtctgttgacgtgaagaaaagaagatgggtgaccttctgctctgccgagtggcctaccttcaacgttggatggcccagagatggcacctttaacagagacctgatcacccaggtgaagatcaaggtgttcagccctggcccccatggccaccctgatcaggtgccttacatcgtgacctgggaggccctggccttcgacccccctccatgggtcaaacctttcgtgcaccccaagcctccaccccctctgccacctagcgccccctccctgcccctggagcccccgcggagcacacccccaagatccagcctctatcctgccctgaccccgtccctgggagccaagccgaagcctcaggtgctgtccgacagcggcggaccactgatcgacctgctgacggaagaccctcctccttacagagaccctagacctcctccttctgatagagacggcaacggcggcgaggccacgcccgccggtgaagcccctgatccctctcccatggccagcagactcagaggccggagagaaccccctgtcgccgattccacaacatcccaagccttccccctgcgggccggcggcaacggccaactgcagtactggcccttttccagcagcgacctgtacaactggaagaacaataatcctagtttttctgaggaccctggcaaactgacagctctgatcgagagtgtgctgattacacaccagcccacctgggatgattgccagcaactgctgggcaccctgctgacaggcgaggaaaagcagagagtgctgctggaggccagaaaggccgtgcggggcgatgatggccggcctacccaactgcctaatgaggtggacgccgcctttcctctggagagacctgattgggactacacaacccaagccggacgaaaccatctggtccactaccggcaactgctgctggccggcctgcagaacgcgggaagaagccccaccaacctggctaaagtgaaaggcataacgcaagggcctaatgagtcaccctccgcctttctggaacggctgaaggaagcttataggcggtacaccccctacgaccctgaggaccctggccaggagaccaacgtgagcatgagcttcatctggcagagcgcccctgacataggaaggaagctggagcggctggaggatctgaagaacaagacactgggcgatctagtgagagaggctgagaagatcttcaacaagagagagacccctgaggagagagaagaacggatcagaagagagacagaggaaaaggaagaaagaagaagaacagaggacgagcagaaggaaaaagagagagacagaagacgccacagagagatgagcaagctgctggccaccgtagtttcgggacagaaacaggaccggcagggcggagaaagacggcggagccaactggaccgggaccagtgcgcctactgcaaggagaagggccactgggccaaggactgtcctaaaaagccaagaggccctcggggacctagacctcaaacaagtctgctgaccctggacgacgacccaggcttcttcaacgtggaaatccctgagttccctttctaccctacctgtaatgtgtgcaccgccgacgtgaacgtcaccatcaacttcgacgtgggaggcaagaagcaccaactggacttggacttcggccaactgaccccccacaccaaggccgtgtaccagcctagaggcgctttcggcggcagcgagaacgccaccaacctgttcctgctggagctgctgggagccggcgagctggccctgaccatgagaagcaaaaagcttcctatcaacgtgacgaccggcgaagagcagcaggtgagcctggaaagcgtggacgtctacttccaggatgtgttcggcaccatgtggtgccaccacgccgagatgcagaaccctgtgtacctgatccctgagaccgtgccctacatcaagtgggacaactgcaactccacaaacatcaccgctgtggtgagggcccagggcctggacgtgaccctgcccctgagcctgcctacttctgcccaagacagcaacttctcagtgaagacccaaatgctgggaaacgagatcgacatcgagtgcatcatggaagatggcgagattagtcaggtgctgcctggcgacaacaagttcaacataacatgcagcggatacgagagccatgtgcctagtgggggtatcctgacttcaactagccctgtggccactccgatccccggaaccggctacgcctattctctgagacttacaccaagacctgtgagcagattcctgggcaacaacagcatcctgtacgtgttctacagcggcaatggccccaaggcgtccggcggcgattactgtatccagagcaacatcgtgttcagcgatgagatccctgccagccaagatatgcccaccaacaccaccgacatcacatacgtgggcgataacgccacctacagcgtgcctatggtgaccagcgaggacgccaactctcctaatgtgaccgtgacagccttctgggcctggcctaacaacaccgagacggatttcaagtgcaagtggacactaacatctggaacccctagcggttgtgagaacatcagcggggccttcgccagcaacagaacctttgacatcaccgtctcaggtctgggcaccgccccaaagacgctgatcatcaccagaacagccaccaacgctaccaccacaacacataaggtgattttcagcaaagccccggaatctaccaccacctctcccaccctgaacacgacagggtttgccgatcccaatactaccaccggcctgccatctagcacccatgtgcctaccaacctgacagccccagcaagcaccgggccaacggtgtcgaccgccgacgtgaccagccctacgccagccggcacaacctctggagccagcccagtgacccctagccctagccctcgggacaacggcacagaaagcaaggcccccgacatgaccagcagcaccagccccgtgaccacccctacacccaacgctacatctcccacaccagccgtgacaacacccacacccaatgctaccagccctacccctgccgtgaccacccccaccccaaatgccacctctcctaccctcggcaagaccagccccacctctgctgttaccaccccgacacctaatgccacttcacctacactgggcaagaccagccctaccagcgccgttacaactcctacccccaacgccaccagcccgactctgggcaaaactagccccaccagtgccgtgactacaccgaccccgaatgccaccggccctaccgttggagaaaccagccctcaggctaacgcgaccaaccacacactgggcggcacaagccctaccccagtggtgacatcccagcccaagaacgccacatctgcggtgaccaccggccagcacaacatcacctccagcagcacctccagcatgagcctcagaccaagctccatccctgagacactcagccctagcacaagcgacaactctacctcacacatgcctctgctgaccagcgcccaccctaccggtggcgagaacatcacccaggtgaccccagcttcgatcagcacgcaccatgtgagcacatcttcacctgctcctagacctggcaccacatcccaggcctctggccctggcaacagcagcacctctaccaagcctggcgaagtgaacgtaacaaagggcacacctccccagaacgctacctcgccccaagctcccagcgggcagaagaccgcggttccaacagtgacttctacaggaggcaaggccaatagcaccacaggcggaaagcacaccaccggccacggcgcacggacatctacagaacctacaactgactacggcggcgacagcaccacccccagacctagatacaacgccaccacctacctgcctccatctacaagcagcaagctgagacccagatggaccttcacgtcccctcctgtgaccaccgctcaggctacagtccctgtgccccctaccagccagcctcgcttcagctga

SEQ ID NO: 12 - RNA, 4125 nt, synthetic construct
  source 1..4125: mol_type = other RNA
atgggccagacagtgaccacccctctgtccctgaccctgggccactggaaggacgtggaaagaatcgcccacaaccagtctgttgacgtgaagaaaagaagatgggtgaccttctgctctgccgagtggcctaccttcaacgttggatggcccagagatggcacctttaacagagacctgatcacccaggtgaagatcaaggtgttcagccctggcccccatggccaccctgatcaggtgccttacatcgtgacctgggaggccctggccttcgacccccctccatgggtcaaacctttcgtgcaccccaagcctccaccccctctgccacctagcgccccctccctgcccctggagcccccgcggagcacacccccaagatccagcctctatcctgccctgaccccgtccctgggagccaagccgaagcctcaggtgctgtccgacagcggcggaccactgatcgacctgctgacggaagaccctcctccttacagagaccctagacctcctccttctgatagagacggcaacggcggcgaggccacgcccgccggtgaagcccctgatccctctcccatggccagcagactcagaggccggagagaaccccctgtcgccgattccacaacatcccaagccttccccctgcgggccggcggcaacggccaactgcagtactggcccttttccagcagcgacctgtacaactggaagaacaataatcctagtttttctgaggaccctggcaaactgacagctctgatcgagagtgtgctgattacacaccagcccacctgggatgattgccagcaactgctgggcaccctgctgacaggcgaggaaaagcagagagtgctgctggaggccagaaaggccgtgcggggcgatgatggccggcctacccaactgcctaatgaggtggacgccgcctttcctctggagagacctgattgggactacacaacccaagccggacgaaaccatctggtccactaccggcaactgctgctggccggcctgcagaacgcgggaagaagccccaccaacctggctaaagtgaaaggcataacgcaagggcctaatgagtcaccctccgcctttctggaacggctgaaggaagcttataggcggtacaccccctacgaccctgaggaccctggccaggagaccaacgtgagcatgagcttcatctggcagagcgcccctgacataggaaggaagctggagcggctggaggatctgaagaacaagacactgggcgatctagtgagagaggctgagaagatcttcaacaagagagagacccctgaggagagagaagaacggatcagaagagagacagaggaaaaggaagaaagaagaagaacagaggacgagcagaaggaaaaagagagagacagaagacgccacagagagatgagcaagctgctggccaccgtagtttcgggacagaaacaggaccggcagggcggagaaagacggcggagccaactggaccgggaccagtgcgcctactgcaaggagaagggccactgggccaaggactgtcctaaaaagccaagaggccctcggggacctagacctcaaacaagtctgctgaccctggacgacgacccaggcttcttcaacgtggaaatccctgagttccctttctaccctacctgtaatgtgtgcaccgccgacgtgaacgtcaccatcaacttcgacgtgggaggcaagaagcaccaactggacttggacttcggccaactgaccccccacaccaaggccgtgtaccagcctagaggcgctttcggcggcagcgagaacgccaccaacctgttcctgctggagctgctgggagccggcgagctggccctgaccatgagaagcaaaaagcttcctatcaacgtgacgaccggcgaagagcagcaggtgagcctggaaagcgtggacgtctacttccaggatgtgttcggcaccatgtggtgccaccacgccgagatgcagaaccctgtgtacctgatccctgagaccgtgccctacatcaagtgggacaactgcaactccacaaacatcaccgctgtggtgagggcccagggcctggacgtgaccctgcccctgagcctgcctacttctgcccaagacagcaacttctcagtgaagacccaaatgctgggaaacgagatcgacatcgagtgcatcatggaagatggcgagattagtcaggtgctgcctggcgacaacaagttcaacataacatgcagcggatacgagagccatgtgcctagtgggggtatcctgacttcaactagccctgtggccactccgatccccggaaccggctacgcctattctctgagacttacaccaagacctgtgagcagattcctgggcaacaacagcatcctgtacgtgttctacagcggcaatggccccaaggcgtccggcggcgattactgtatccagagcaacatcgtgttcagcgatgagatccctgccagccaagatatgcccaccaacaccaccgacatcacatacgtgggcgataacgccacctacagcgtgcctatggtgaccagcgaggacgccaactctcctaatgtgaccgtgacagccttctgggcctggcctaacaacaccgagacggatttcaagtgcaagtggacactaacatctggaacccctagcggttgtgagaacatcagcggggccttcgccagcaacagaacctttgacatcaccgtctcaggtctgggcaccgccccaaagacgctgatcatcaccagaacagccaccaacgctaccaccacaacacataaggtgattttcagcaaagccccggaatctaccaccacctctcccaccctgaacacgacagggtttgccgatcccaatactaccaccggcctgccatctagcacccatgtgcctaccaacctgacagccccagcaagcaccgggccaacggtgtcgaccgccgacgtgaccagccctacgccagccggcacaacctctggagccagcccagtgacccctagccctagccctcgggacaacggcacagaaagcaaggcccccgacatgaccagcagcaccagccccgtgaccacccctacacccaacgctacatctcccacaccagccgtgacaacacccacacccaatgctaccagccctacccctgccgtgaccacccccaccccaaatgccacctctcctaccctcggcaagaccagccccacctctgctgttaccaccccgacacctaatgccacttcacctacactgggcaagaccagccctaccagcgccgttacaactcctacccccaacgccaccagcccgactctgggcaaaactagccccaccagtgccgtgactacaccgaccccgaatgccaccggccctaccgttggagaaaccagccctcaggctaacgcgaccaaccacacactgggcggcacaagccctaccccagtggtgacatcccagcccaagaacgccacatctgcggtgaccaccggccagcacaacatcacctccagcagcacctccagcatgagcctcagaccaagctccatccctgagacactcagccctagcacaagcgacaactctacctcacacatgcctctgctgaccagcgcccaccctaccggtggcgagaacatcacccaggtgaccccagcttcgatcagcacgcaccatgtgagcacatcttcacctgctcctagacctggcaccacatcccaggcctctggccctggcaacagcagcacctctaccaagcctggcgaagtgaacgtaacaaagggcacacctccccagaacgctacctcgccccaagctcccagcgggcagaagaccgcggttccaacagtgacttctacaggaggcaaggccaatagcaccacaggcggaaagcacaccaccggccacggcgcacggacatctacagaacctacaactgactacggcggcgacagcaccacccccagacctagatacaacgccaccacctacctgcctccatctacaagcagcaagctgagacccagatggaccttcacgtcccctcctgtgaccaccgctcaggctacagtccctgtgccccctaccagccagcctcgcttcagctga

SEQ ID NO: 13 - protein, 907 aa, synthetic construct
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTQMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPRDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSIPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 14 - DNA, 2724 bp, synthetic construct
  source 1..2724: mol_type = other DNA
atggaggcagccttgcttgtgtgtcagtacaccatccagagcctgatccatctcacgggtgaagatcctggttttttcaatgttgagattccggaattcccattttaccccacatgcaatgtttgcacggcagatgtcaatgtaactatcaatttcgatgtcgggggcaaaaagcatcaacttgatcttgactttggccagctgacaccccatacgaaggctgtctaccaacctcgaggtgcatttggtggctcagaaaatgccaccaatctctttctactggagctccttggtgcaggagaattggctctaactatgcggtctaagaagcttccaattaacgtcaccaccggagaggagcaacaagtaagcctggaatctgtagatgtctactttcaagatgtgtttggaaccatgtggtgccaccatgcagaaatgcaaaaccccgtgtacctgataccagaaacagtgccatacataaagtgggataactgtaattctaccaatataacggcagtagtgagggcacaggggctggatgtcacgctacccttaagtttgccaacgtcagctcaagactcgaatttcagcgtaaaaacacaaatgctcggtaatgagatagatattgagtgtattatggaggatggcgaaatttcacaagttctgcccggagacaacaaatttaacatcacctgcagtggatacgagagccatgttcccagcggcggaattctcacatcaacgagtcccgtggccaccccaatacctggtacagggtatgcatacagcctgcgtctgacaccacgtccagtgtcacgatttcttggcaataacagtatcctgtacgtgttttactctgggaatggaccgaaggcgagcgggggagattactgcattcagtccaacattgtgttctctgatgagattccagcttcacaggacatgccgacaaacaccacagacatcacatatgtgggtgacaatgctacctattcagtgccaatggtcacttctgaggacgcaaactcgccaaatgttacagtgactgccttttgggcctggccaaacaacactgaaactgactttaagtgcaaatggactctcacctcggggacaccttcgggttgtgaaaatatttctggtgcatttgcgagcaatcggacatttgacattactgtctcgggtcttggcacggcccccaagacactcattatcacacgaacggctaccaatgccaccacaacaacccacaaggttatattctccaaggcacccgagagcaccaccacctcccctaccttgaatacaactggatttgctgatcccaatacaacgacaggtctacccagctctactcacgtgcctaccaacctcaccgcacctgcaagcacaggccccactgtatccaccgcggatgtcaccagcccaacaccagccggcacaacgtcaggcgcatcaccggtgacaccaagtccatctccaagggacaacggcacagaaagtaaggcccccgacatgaccagctccacctcaccagtgactaccccaaccccaaatgccaccagccccaccccagcagtgactaccccaaccccaaatgccaccagccccaccccagcagtgactaccccaaccccaaatgccaccagccccaccttgggaaaaacaagtcctacctcagcagtgactaccccaaccccaaatgccaccagccccaccttgggaaaaacaagccccacctcagcagtgactaccccaaccccaaatgccaccagccccaccttgggaaaaacaagccccacctcagcagtgactaccccaaccccaaatgccaccggccctactgtgggagaaacaagtccacaggcaaatgccaccaaccacaccttaggaggaacaagtcccaccccagtagttaccagccaaccaaaaaatgcaaccagtgctgttaccacaggccaacataacataacttcaagttcaacctcttccatgtcactgagacccagttcaatcccagagacactcagcccctccaccagtgacaattcaacgtcacatatgcctttactaacctccgctcacccaacaggtggtgaaaatataacacaggtgacaccagcctctatcagcacacatcatgtgtccaccagttcgccagcaccccgcccaggcaccaccagccaagcgtcaggccctggaaacagttccacatccacaaaaccgggggaggttaatgtcaccaaaggcacgcccccccaaaatgcaacgtcgccccaggcccccagtggccaaaagacggcggttcccacggtcacctcaacaggtggaaaggccaattctaccaccggtggaaagcacaccacaggacatggagcccggacaagtacagagcccaccacagattacggcggtgattcaactacgccaagaccgagatacaatgcgaccacctatctacctcccagcacttctagcaaactgcggccccgctggacttttacgagcccaccggttaccacagcccaagccaccgtgccagtcccgccaacgtcccagcccagattctcaaacctctccatgctagtactgcagtgggcctctctggctgtgctgacccttctgctgctgctggtcatggcggactgcgcctttaggcgtaacttgtctacatcccatacctacaccaccccaccatatgatgacgccgagacctatgtataa

SEQ ID NO: 15 - DNA, 2724 bp, synthetic construct
  source 1..2724: mol_type = other DNA
atggaggccgctctgctggtctgccagtacactattcagtctctgattcacctgaccggggaagatcctgggtttttcaatgtggagatccctgagtttccattctacccaacatgcaacgtgtgcaccgccgacgtgaacgtgaccatcaattttgatgtgggcggcaagaagcaccagctagacctggattttggccagctaacacctcacaccaaggccgtgtatcagccacgcggcgcgtttggcggcagcgagaacgccacaaacctgtttctgctggagctgctgggcgcgggcgagctggccctgaccatgagaagcaagaagctgccaatcaacgtgaccaccggcgaggagcagcaggtgtccctggagtccgtggacgtgtacttccaggacgtgttcggcacaatgtggtgccaccacgccgagatgcagaacccagtgtacctgatcccagagaccgtgccatacatcaagtgggacaactgcaactccaccaatatcacagccgtggtgcgggcccagggcctggatgtgacactgcctctgagcctgcctacatctgcccaggacagcaacttttccgtgaagacccagatgctgggcaacgagatcgacatcgagtgcatcatggaggatggcgagatctcccaggtgctgcctggcgacaataagttcaacatcacatgtagcggctacgagagccacgtaccttccggcggcatcctgacctctacaagcccagtggccaccccaatccccggcaccggctatgcctactctctgaggctgacacctcggcccgtgagcagattcctgggcaacaactccatcctgtacgtgttctactccggcaatggccctaaggccagcggcggcgactactgtatccagagcaacatcgtgttttccgacgagatccctgcctctcaggatatgcccaccaataccaccgacatcacatacgtgggcgacaatgccacatatagcgtgccaatggtgaccagcgaggacgccaatagccctaacgtgacagtgaccgccttttgggcctggcccaacaatacagagaccgatttcaagtgtaagtggaccctgaccagcggcaccccttccggctgcgagaatatcagcggcgcgtttgcctctaaccggacattcgacatcacagtgtccggcctgggcacagcccccaagaccctgatcatcaccagaaccgccacaaacgccacaacaacaacccacaaggtaatcttttccaaggccccagagtctaccacaacatcccccaccctgaataccacaggcttcgccgaccctaataccacaaccggcctgccttctagcacccacgtaccaacaaacctgaccgccccagcctccacaggcccaaccgtgtccacagccgacgtgacctcccctacccccgccggcaccacatccggcgcgagcccagtgaccccatccccttcccccagggataatggcacagagagcaaggcccccgatatgacatcctctacaagccctgtgaccacccctacccccaacgccaccagccccaccccagccgtgaccaccccaacacccaacgccacctcccctacacctgccgtgacaaccccaacacctaatgccacatccccaacactgggcaagacctccccaacatccgccgtgacaacaccaacccccaatgccacctctccaacactgggcaagacatcccctacctccgccgtgaccacacctaccccaaacgccacatcccccacactgggcaagaccagcccaacatctgccgtgacaaccccaaccccaaatgccacaggccccaccgtgggcgagacaagccctcaggccaatgccacaaatcacaccctgggcggcacctcccctacacccgtggtgacatctcagcccaagaatgccacatctgccgtgacaacaggccagcacaatatcacatcctctagcacatcctccatgtccctgcgcccctcttccatcccagagacactgagcccaagcacctccgacaactccacatctcacatgccactgctgacctctgcccaccccacaggcggcgagaacatcacccaggtgacacctgcctccatcagcacccaccacgtatctaccagctcccctgccccaagaccaggcacaacaagccaggcctccggcccaggcaattcttccacatctaccaagccaggcgaggtgaacgtgaccaagggcacccccccacagaatgccacctcccctcaggccccttccggccagaagacagccgtgccaaccgtgacatccaccggcggcaaggccaattctacaacaggcggcaagcacaccaccggccacggcgcgagaaccagcaccgagcctacaaccgactacggcggcgattccacaaccccaagacccagatacaacgccaccacctacctgcccccttccacaagctctaagctgagaccaagatggacattcacatcccccccagtgaccacagcccaggccacagtgcccgtgccccccaccagccagccacggttctccaacctgtctatgctggtgctgcagtgggcctctctggccgtgctgacactgctgctgctgctggtaatggccgattgcgccttccggcggaatctgtcaacaagccacacctacactacacctccttacgatgatgccgaaacttatgtctga

SEQ ID NO: 16 - RNA, 2724 nt, synthetic construct
  source 1..2724: mol_type = other RNA
atggaggccgctctgctggtctgccagtacactattcagtctctgattcacctgaccggggaagatcctgggtttttcaatgtggagatccctgagtttccattctacccaacatgcaacgtgtgcaccgccgacgtgaacgtgaccatcaattttgatgtgggcggcaagaagcaccagctagacctggattttggccagctaacacctcacaccaaggccgtgtatcagccacgcggcgcgtttggcggcagcgagaacgccacaaacctgtttctgctggagctgctgggcgcgggcgagctggccctgaccatgagaagcaagaagctgccaatcaacgtgaccaccggcgaggagcagcaggtgtccctggagtccgtggacgtgtacttccaggacgtgttcggcacaatgtggtgccaccacgccgagatgcagaacccagtgtacctgatcccagagaccgtgccatacatcaagtgggacaactgcaactccaccaatatcacagccgtggtgcgggcccagggcctggatgtgacactgcctctgagcctgcctacatctgcccaggacagcaacttttccgtgaagacccagatgctgggcaacgagatcgacatcgagtgcatcatggaggatggcgagatctcccaggtgctgcctggcgacaataagttcaacatcacatgtagcggctacgagagccacgtaccttccggcggcatcctgacctctacaagcccagtggccaccccaatccccggcaccggctatgcctactctctgaggctgacacctcggcccgtgagcagattcctgggcaacaactccatcctgtacgtgttctactccggcaatggccctaaggccagcggcggcgactactgtatccagagcaacatcgtgttttccgacgagatccctgcctctcaggatatgcccaccaataccaccgacatcacatacgtgggcgacaatgccacatatagcgtgccaatggtgaccagcgaggacgccaatagccctaacgtgacagtgaccgccttttgggcctggcccaacaatacagagaccgatttcaagtgtaagtggaccctgaccagcggcaccccttccggctgcgagaatatcagcggcgcgtttgcctctaaccggacattcgacatcacagtgtccggcctgggcacagcccccaagaccctgatcatcaccagaaccgccacaaacgccacaacaacaacccacaaggtaatcttttccaaggccccagagtctaccacaacatcccccaccctgaataccacaggcttcgccgaccctaataccacaaccggcctgccttctagcacccacgtaccaacaaacctgaccgccccagcctccacaggcccaaccgtgtccacagccgacgtgacctcccctacccccgccggcaccacatccggcgcgagcccagtgaccccatccccttcccccagggataatggcacagagagcaaggcccccgatatgacatcctctacaagccctgtgaccacccctacccccaacgccaccagccccaccccagccgtgaccaccccaacacccaacgccacctcccctacacctgccgtgacaaccccaacacctaatgccacatccccaacactgggcaagacctccccaacatccgccgtgacaacaccaacccccaatgccacctctccaacactgggcaagacatcccctacctccgccgtgaccacacctaccccaaacgccacatcccccacactgggcaagaccagcccaacatctgccgtgacaaccccaaccccaaatgccacaggccccaccgtgggcgagacaagccctcaggccaatgccacaaatcacaccctgggcggcacctcccctacacccgtggtgacatctcagcccaagaatgccacatctgccgtgacaacaggccagcacaatatcacatcctctagcacatcctccatgtccctgcgcccctcttccatcccagagacactgagcccaagcacctccgacaactccacatctcacatgccactgctgacctctgcccaccccacaggcggcgagaacatcacccaggtgacacctgcctccatcagcacccaccacgtatctaccagctcccctgccccaagaccaggcacaacaagccaggcctccggcccaggcaattcttccacatctaccaagccaggcgaggtgaacgtgaccaagggcacccccccacagaatgccacctcccctcaggccccttccggccagaagacagccgtgccaaccgtgacatccaccggcggcaaggccaattctacaacaggcggcaagcacaccaccggccacggcgcgagaaccagcaccgagcctacaaccgactacggcggcgattccacaaccccaagacccagatacaacgccaccacctacctgcccccttccacaagctctaagctgagaccaagatggacattcacatcccccccagtgaccacagcccaggccacagtgcccgtgccccccaccagccagccacggttctccaacctgtctatgctggtgctgcagtgggcctctctggccgtgctgacactgctgctgctgctggtaatggccgattgcgccttccggcggaatctgtcaacaagccacacctacactacacctccttacgatgatgccgaaacttatgtctga

SEQ ID NO: 17 - protein, 1353 aa, synthetic construct
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 18 - DNA, 4062 bp, synthetic construct
  source 1..4062: mol_type = other DNA
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

SEQ ID NO: 19 - DNA, 4062 bp, synthetic construct
  source 1..4062: mol_type = other DNA
atgatccacagcgtgttcctgctgatgtttctgctgacacctaccgagtcctacgtggatgtgggcccagactctgtgaagagcgcctgcatcgaggtggacatccagcagacattctttgacaagacctggcccagacccatcgacgtgagcaaggcagacggaatcatctacccacagggacgcacatatagcaacatcacaatcacctaccagggcctgttcccttatcagggcgaccacggcgatatgtacgtgtatagcgccggccacgcaaccggcaccacaccacagaagctgtttgtggccaattattcccaggacgtgaagcagttcgccaacggatttgtggtgcggatcggggccgccgccaacagcacaggcaccgtgatcatctctcccagcacatccgccaccatcagaaagatctaccctgcctttatgctgggcagctccgtgggcaacttctccgatggcaagatgggcaggttctttaatcacacactggtgctgctgccagacggatgcggcaccctgctgagggccttctactgtatcctggagccccgctctggaaatcactgccctgccggcaactcctacacctcttttgccacatatcacacccctgccacagactgttccgatggcaattataaccggaatgccagcctgaactccttcaaggagtactttaatctgagaaactgcaccttcatgtacacatataatatcaccgaggatgagatcctggagtggttcggcatcacacagaccgcccagggcgtgcacctgttttctagcagatacgtggatctgtatggcggcaacatgttccagtttgccacactgccagtgtatgacaccatcaagtactatagcatcatcccccactctatccggagcatccagtccgacagaaaggcctgggccgccttctacgtgtataagctgcagcccctgaccttcctgctggatttttccgtggacggctacatccggagagccatcgattgcggctttaacgacctgtctcagctccactgttcttatgagagcttcgatgtggagtctggcgtgtacagcgtgtcctcttttgaggccaagccatctggcagcgtggtggagcaggcagagggagtggagtgcgacttctccccactgctgtctggcacaccacctcaggtgtataatttcaagaggctggtgtttacaaactgtaattacaacctgaccaagctgctgtccctgttctctgtgaacgactttacctgcagccagatctcccctgccgccatcgcctccaattgttatagctccctgatcctggattacttctcttatcccctgtctatgaagagcgacctgtccgtgtctagcgccggccctatcagccagtttaattacaagcagtccttctctaaccccacatgcctgatcctggccaccgtgcctcacaacctgaccacaatcacaaagccactgaagtactcctatatcaataagtgcagcaggctgctgtccgacgatcgcaccgaggtgcctcagctcgtgaacgccaaccagtactctccatgcgtgagcatcgtgccatccaccgtgtgggaggacggcgattactatagaaagcagctcagcccactggagggaggaggatggctggtggccagcggctccacagtggccatgaccgagcagctccagatgggcttcggcatcacagtgcagtacggcacagataccaatagcgtgtgccccaagctggagtttgccaacgacaccaagatcgcctcccagctcggcaattgcgtggagtactccctgtatggcgtgtctggcagaggcgtgttccagaactgtacagccgtgggcgtgcggcagcagcggttcgtgtacgatgcctatcagaacctggtgggctactatagcgacgatggcaattactattgcctgagggcatgcgtgagcgtgcccgtgagcgtgatctacgacaaggagacaaagacccacgccaccctgttcggctccgtggcctgcgagcacatctcctctacaatgtctcagtattctaggagcacccgctctatgctgaagaggcgcgacagcacatacggaccactgcagacccctgtgggatgcgtgctgggcctggtgaacagcagcctgtttgtggaggattgcaagctgccactgggccagtctctgtgcgcactgccagacacccccagcacactgaccccacggtctgtgagaagcgtgcccggagagatgagactggccagcatcgccttcaatcaccctatccaggtggatcagctcaacagcagctactttaagctgagcatcccaacaaacttctcctttggcgtgacccaggagtatatccagaccacaatccagaaggtgaccgtggactgcaagcagtacgtgtgcaatggcttccagaagtgcgagcagctcctgagggagtatggccagttttgttccaagatcaatcaggccctgcacggagccaacctgaggcaggacgattccgtgagaaacctgttcgcctctgtgaagtcctctcagagctcccctatcatcccaggcttcggcggcgacttcaacctgaccctgctggagcccgtgtccatctctaccggcagcaggtccgcccgcagcgccatcgaggatctgctgtttgacaaggtgaccatcgccgacccaggctacatgcagggctatgacgattgcatgcagcagggaccagcctccgcccgcgatctgatctgtgcccagtacgtggccggctataaggtgctgccacccctgatggacgtgaacatggaggccgcctatacatctagcctgctgggcagcatcgcaggagtgggatggaccgccggcctgtcctctttcgccgcaatcccttttgcccagtctatcttctaccggctgaacggcgtgggcatcacacagcaggtgctgagcgagaatcagaagctgatcgccaataagttcaaccaggccctgggggccatgcagaccggctttaccacaaccaacgaggccttccagaaggtgcaggatgccgtgaacaataacgcacaggccctgtccaagctggcctccgagctgtctaataccttcggggccatcagcgccagcatcggcgacatcatccagcgcctggacgtgctggagcaggatgcccagatcgacaggctgatcaatggccgcctgacaaccctgaacgcctttgtggcacagcagctcgtgcggagcgagtctgccgccctgagcgcccagctcgccaaggacaaggtgaacgagtgcgtgaaggcccagagcaagcggtccggcttttgtggccagggcacccacatcgtgtccttcgtggtgaatgcccctaacggcctgtactttatgcatgtgggctactatccaagcaaccacatcgaggtggtgtccgcctatggcctgtgcgatgccgccaatcctacaaactgtatcgccccagtgaatggctacttcatcaagaccaataacacacggatcgtggacgagtggtcctacaccggcagctccttttatgcccccgagcctatcacatctctgaacaccaagtacgtggccccacaggtgacatatcagaatatcagcaccaacctgcctccacccctgctgggcaattccaccggcatcgacttccaggatgagctggacgagttctttaagaatgtgagcacatccatccccaactttggcagcctgacccagatcaacacaaccctgctggatctgacatacgagatgctgtctctgcagcaggtggtgaaggccctgaatgagagctacatcgacctgaaggagctgggcaattatacctactataacaagtggccttggtacatctggctgggcttcatcgcaggcctggtggccctggccctgtgcgtgttctttatcctgtgctgtacaggctgcggcaccaattgtatgggcaagctgaagtgtaaccggtgctgtgatagatacgaggagtatgacctggagccacacaaggtgcatgtgcactga

SEQ ID NO: 20 - RNA, 4062 nt, synthetic construct
  source 1..4062: mol_type = other RNA
atgatccacagcgtgttcctgctgatgtttctgctgacacctaccgagtcctacgtggatgtgggcccagactctgtgaagagcgcctgcatcgaggtggacatccagcagacattctttgacaagacctggcccagacccatcgacgtgagcaaggcagacggaatcatctacccacagggacgcacatatagcaacatcacaatcacctaccagggcctgttcccttatcagggcgaccacggcgatatgtacgtgtatagcgccggccacgcaaccggcaccacaccacagaagctgtttgtggccaattattcccaggacgtgaagcagttcgccaacggatttgtggtgcggatcggggccgccgccaacagcacaggcaccgtgatcatctctcccagcacatccgccaccatcagaaagatctaccctgcctttatgctgggcagctccgtgggcaacttctccgatggcaagatgggcaggttctttaatcacacactggtgctgctgccagacggatgcggcaccctgctgagggccttctactgtatcctggagccccgctctggaaatcactgccctgccggcaactcctacacctcttttgccacatatcacacccctgccacagactgttccgatggcaattataaccggaatgccagcctgaactccttcaaggagtactttaatctgagaaactgcaccttcatgtacacatataatatcaccgaggatgagatcctggagtggttcggcatcacacagaccgcccagggcgtgcacctgttttctagcagatacgtggatctgtatggcggcaacatgttccagtttgccacactgccagtgtatgacaccatcaagtactatagcatcatcccccactctatccggagcatccagtccgacagaaaggcctgggccgccttctacgtgtataagctgcagcccctgaccttcctgctggatttttccgtggacggctacatccggagagccatcgattgcggctttaacgacctgtctcagctccactgttcttatgagagcttcgatgtggagtctggcgtgtacagcgtgtcctcttttgaggccaagccatctggcagcgtggtggagcaggcagagggagtggagtgcgacttctccccactgctgtctggcacaccacctcaggtgtataatttcaagaggctggtgtttacaaactgtaattacaacctgaccaagctgctgtccctgttctctgtgaacgactttacctgcagccagatctcccctgccgccatcgcctccaattgttatagctccctgatcctggattacttctcttatcccctgtctatgaagagcgacctgtccgtgtctagcgccggccctatcagccagtttaattacaagcagtccttctctaaccccacatgcctgatcctggccaccgtgcctcacaacctgaccacaatcacaaagccactgaagtactcctatatcaataagtgcagcaggctgctgtccgacgatcgcaccgaggtgcctcagctcgtgaacgccaaccagtactctccatgcgtgagcatcgtgccatccaccgtgtgggaggacggcgattactatagaaagcagctcagcccactggagggaggaggatggctggtggccagcggctccacagtggccatgaccgagcagctccagatgggcttcggcatcacagtgcagtacggcacagataccaatagcgtgtgccccaagctggagtttgccaacgacaccaagatcgcctcccagctcggcaattgcgtggagtactccctgtatggcgtgtctggcagaggcgtgttccagaactgtacagccgtgggcgtgcggcagcagcggttcgtgtacgatgcctatcagaacctggtgggctactatagcgacgatggcaattactattgcctgagggcatgcgtgagcgtgcccgtgagcgtgatctacgacaaggagacaaagacccacgccaccctgttcggctccgtggcctgcgagcacatctcctctacaatgtctcagtattctaggagcacccgctctatgctgaagaggcgcgacagcacatacggaccactgcagacccctgtgggatgcgtgctgggcctggtgaacagcagcctgtttgtggaggattgcaagctgccactgggccagtctctgtgcgcactgccagacacccccagcacactgaccccacggtctgtgagaagcgtgcccggagagatgagactggccagcatcgccttcaatcaccctatccaggtggatcagctcaacagcagctactttaagctgagcatcccaacaaacttctcctttggcgtgacccaggagtatatccagaccacaatccagaaggtgaccgtggactgcaagcagtacgtgtgcaatggcttccagaagtgcgagcagctcctgagggagtatggccagttttgttccaagatcaatcaggccctgcacggagccaacctgaggcaggacgattccgtgagaaacctgttcgcctctgtgaagtcctctcagagctcccctatcatcccaggcttcggcggcgacttcaacctgaccctgctggagcccgtgtccatctctaccggcagcaggtccgcccgcagcgccatcgaggatctgctgtttgacaaggtgaccatcgccgacccaggctacatgcagggctatgacgattgcatgcagcagggaccagcctccgcccgcgatctgatctgtgcccagtacgtggccggctataaggtgctgccacccctgatggacgtgaacatggaggccgcctatacatctagcctgctgggcagcatcgcaggagtgggatggaccgccggcctgtcctctttcgccgcaatcccttttgcccagtctatcttctaccggctgaacggcgtgggcatcacacagcaggtgctgagcgagaatcagaagctgatcgccaataagttcaaccaggccctgggggccatgcagaccggctttaccacaaccaacgaggccttccagaaggtgcaggatgccgtgaacaataacgcacaggccctgtccaagctggcctccgagctgtctaataccttcggggccatcagcgccagcatcggcgacatcatccagcgcctggacgtgctggagcaggatgcccagatcgacaggctgatcaatggccgcctgacaaccctgaacgcctttgtggcacagcagctcgtgcggagcgagtctgccgccctgagcgcccagctcgccaaggacaaggtgaacgagtgcgtgaaggcccagagcaagcggtccggcttttgtggccagggcacccacatcgtgtccttcgtggtgaatgcccctaacggcctgtactttatgcatgtgggctactatccaagcaaccacatcgaggtggtgtccgcctatggcctgtgcgatgccgccaatcctacaaactgtatcgccccagtgaatggctacttcatcaagaccaataacacacggatcgtggacgagtggtcctacaccggcagctccttttatgcccccgagcctatcacatctctgaacaccaagtacgtggccccacaggtgacatatcagaatatcagcaccaacctgcctccacccctgctgggcaattccaccggcatcgacttccaggatgagctggacgagttctttaagaatgtgagcacatccatccccaactttggcagcctgacccagatcaacacaaccctgctggatctgacatacgagatgctgtctctgcagcaggtggtgaaggccctgaatgagagctacatcgacctgaaggagctgggcaattatacctactataacaagtggccttggtacatctggctgggcttcatcgcaggcctggtggccctggccctgtgcgtgttctttatcctgtgctgtacaggctgcggcaccaattgtatgggcaagctgaagtgtaaccggtgctgtgatagatacgaggagtatgacctggagccacacaaggtgcatgtgcactga

SEQ ID NO: 21 - protein, 795 aa, synthetic construct
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSINVFDLEEIMREFNSYKQRVKYVEDKVVDPLPPYLKGLDDLMSGLGAAGKAVGVAIGAVGGAVASVVEGVATFLKNPFFFIIGLIIGLFLVLRVGIHLCIKLKHTKKRQIYTDIEMNRLGK

SEQ ID NO: 22 - DNA, 2388 bp, synthetic construct
  source 1..2388: mol_type = other DNA
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcattctcgatccggttcagtctctcaacgcgtaacttcttcccaaacggtcagccatggtgttaacgagaccatctacaacactaccctcaagtacggagatgtggtgggggtcaacaccaccaagtacccctatcgcgtgtgttctatggcacagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagtcacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaagtatccctatcattttttcgcaacttccacgggtgatgtggttgacatttctcctttctacaacggaactaatcgcaatgccagctattttggagaaaacgccgacaagtttttcatttttccgaactacactatcgtctccgactttggaagaccgaattctgcgttagagacccacaggttggtggcttttcttgaacgtgcggactcagtgatctcctgggatatacaggacgagaagaatgttacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgtgtacgtgatgaggccataaataagttacagcagattttcaatacttcatacaatcaaacatatgaaaaatatggaaacgtgtccgtctttgaaaccactggtggtttggtggtgttctggcaaggtatcaagcaaaaatctctggtggaactcgaacgtttggccaaccgctccagtctgaatcttactcataatagaaccaaaagaagtacagatggcaacaatgcaactcatttatccaacatggagtcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaacttagcaagatcaacccgtcagctattctctcggccatctacaacaaaccgattgccgcgcgtttcatgggtgatgtcctgggtctggccagctgcgtgaccattaaccaaaccagcgtcaaggtgctgcgtgatatgaatgtgaaggaatcgccaggacgctgctactcacgaccagtggtcatctttaatttcgccaacagctcgtacgtgcagtacggtcaactgggcgaggataacgaaatcctgttgggcaaccaccgcactgaggaatgtcagcttcccagcctcaagatcttcatcgccggcaactcggcctacgagtacgtggactacctcttcaaacgcatgattgacctcagcagcatctccaccgtcgacagcatgatcgccctagacatcgacccgctggaaaacaccgacttcagggtactggaactttactcgcagaaagaattgcgttccatcaacgtttttgatctcgaggagatcatgcgcgagttcaattcgtataagcagcgggtaaagtacgtggaggacaaggtagtcgacccgctgccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaacccctttttctttatcatagggttaatcattggactattcttggttctccgagttggtatccatctttgcattaaattaaagcacaccaagaaaagacagatttatacagacatagagatgaaccgacttggaaagtaa

SEQ ID NO: 23 - DNA, 2388 bp, synthetic construct
  source 1..2388: mol_type = other DNA
atggagtcaaggatttggtgtctggtcgtctgcgtcaacctgtgcattgtctgcctgggagccgccgtctcatcatcatctacccgaggcacatccgccactcactctcaccatagctcccataccacatccgccgctcactcaagaagcgggtccgtgtctcagagggtcacatctagtcagaccgtgagccatggagtcaacgagacaatctacaatactaccctgaagtatggagacgtggtcggcgtgaacacaactaaatacccctatagggtctgctctatggcccaggggacagatctgatccgatttgaacggaacatcgtgtgcactagcatgaagcctatcaatgaggacctggatgaaggaattatggtggtctacaaacgaaatatcgtggcccatacttttaaggtgagagtctatcagaaagtgctgaccttccggagaagctacgcttatattcacaccacatacctgctggggtccaacaccgagtatgtggcaccccctatgtgggaaatccaccatattaatagtcattcacagtgctactcaagctacagcagagtgatcgctggaaccgtgttcgtcgcataccacagagacagttatgagaacaagacaatgcagctcatgcccgacgattacagtaatacccattcaacaagatatgtgaccgtcaaagatcagtggcactctcgcggcagtacctggctgtaccgagagacatgcaacctgaattgtatggtgacaattactaccgccagaagcaagtacccttatcacttctttgctacctcaacaggggacgtggtcgacatcagccccttctacaacggaacaaaccggaatgcctcctatttcggcgagaacgctgacaaattctttatcttccccaactacactatcgtgagcgatttcggcagacctaacagtgccctggagacccatcggctggtggcatttctggaaagagccgacagcgtgatctcctgggacattcaggatgagaagaatgtgacctgccagctcaccttctgggaggccagcgaaagaaccatcaggtccgaggcagaagattcttaccacttttcctctgcaaaaatgactgccaccttcctgtccaagaaacaggaggtgaacatgagcgactccgcactggattgcgtgcgggacgaagccatcaataagctgcagcagatcttcaacacatcttacaaccagacttacgagaagtacggcaacgtgagtgtctttgaaacaactggcgggctggtggtcttctggcaggggatcaagcagaaatctctggtggagctggaacggctggccaatagaagttcactgaacctgactcataatcgcaccaagcgatccacagacggaaacaatgcaactcatctgagcaacatggagtccgtgcacaatctggtctacgcccagctccagttcacttacgacaccctgcgaggctatatcaaccgggccctggctcagattgcagaagcctggtgcgtggatcagaggcgcaccctggaggtctttaaggaactgagcaaaattaacccatctgctatcctgagtgcaatctacaataagcccatcgcagccaggttcatgggggacgtgctgggactggcctcctgcgtcactatcaaccagacctctgtgaaggtcctgcgcgatatgaatgtgaaagagagtcctggcaggtgttattcacgcccagtggtcatcttcaacttcgctaatagctcctacgtgcagtatggccagctcggggaggacaacgaaatcctgctgggaaatcacaggaccgaggaatgtcagctcccaagtctgaagatctttattgccggcaactcagcttacgagtatgtggattacctgttcaaacgcatgatcgacctgtctagtatttcaacagtggatagcatgatcgccctggacattgatcccctggaaaatactgacttcagggtgctggagctgtatagccagaaggaactgcgctccattaacgtgtttgatctggaggaaatcatgagggagttcaattcctacaagcagcgcgtgaaatatgtcgaagataaggtggtcgaccctctgccaccctacctgaaaggcctggacgatctgatgagcgggctgggagctgcaggcaaggcagtgggagtcgccatcggagctgtgggaggcgctgtcgcatccgtggtcgagggagtggctacctttctgaagaacccattctttttcatcatcggcctgatcattgggctgttcctggtgctgagagtcggcatccacctgtgcattaagctgaagcacaccaagaagaggcagatctacaccgatattgaaatgaacagactgggcaagtga

SEQ ID NO: 24 - RNA, 2388 nt, synthetic construct
  source 1..2388: mol_type = other RNA
atggagtcaaggatttggtgtctggtcgtctgcgtcaacctgtgcattgtctgcctgggagccgccgtctcatcatcatctacccgaggcacatccgccactcactctcaccatagctcccataccacatccgccgctcactcaagaagcgggtccgtgtctcagagggtcacatctagtcagaccgtgagccatggagtcaacgagacaatctacaatactaccctgaagtatggagacgtggtcggcgtgaacacaactaaatacccctatagggtctgctctatggcccaggggacagatctgatccgatttgaacggaacatcgtgtgcactagcatgaagcctatcaatgaggacctggatgaaggaattatggtggtctacaaacgaaatatcgtggcccatacttttaaggtgagagtctatcagaaagtgctgaccttccggagaagctacgcttatattcacaccacatacctgctggggtccaacaccgagtatgtggcaccccctatgtgggaaatccaccatattaatagtcattcacagtgctactcaagctacagcagagtgatcgctggaaccgtgttcgtcgcataccacagagacagttatgagaacaagacaatgcagctcatgcccgacgattacagtaatacccattcaacaagatatgtgaccgtcaaagatcagtggcactctcgcggcagtacctggctgtaccgagagacatgcaacctgaattgtatggtgacaattactaccgccagaagcaagtacccttatcacttctttgctacctcaacaggggacgtggtcgacatcagccccttctacaacggaacaaaccggaatgcctcctatttcggcgagaacgctgacaaattctttatcttccccaactacactatcgtgagcgatttcggcagacctaacagtgccctggagacccatcggctggtggcatttctggaaagagccgacagcgtgatctcctgggacattcaggatgagaagaatgtgacctgccagctcaccttctgggaggccagcgaaagaaccatcaggtccgaggcagaagattcttaccacttttcctctgcaaaaatgactgccaccttcctgtccaagaaacaggaggtgaacatgagcgactccgcactggattgcgtgcgggacgaagccatcaataagctgcagcagatcttcaacacatcttacaaccagacttacgagaagtacggcaacgtgagtgtctttgaaacaactggcgggctggtggtcttctggcaggggatcaagcagaaatctctggtggagctggaacggctggccaatagaagttcactgaacctgactcataatcgcaccaagcgatccacagacggaaacaatgcaactcatctgagcaacatggagtccgtgcacaatctggtctacgcccagctccagttcacttacgacaccctgcgaggctatatcaaccgggccctggctcagattgcagaagcctggtgcgtggatcagaggcgcaccctggaggtctttaaggaactgagcaaaattaacccatctgctatcctgagtgcaatctacaataagcccatcgcagccaggttcatgggggacgtgctgggactggcctcctgcgtcactatcaaccagacctctgtgaaggtcctgcgcgatatgaatgtgaaagagagtcctggcaggtgttattcacgcccagtggtcatcttcaacttcgctaatagctcctacgtgcagtatggccagctcggggaggacaacgaaatcctgctgggaaatcacaggaccgaggaatgtcagctcccaagtctgaagatctttattgccggcaactcagcttacgagtatgtggattacctgttcaaacgcatgatcgacctgtctagtatttcaacagtggatagcatgatcgccctggacattgatcccctggaaaatactgacttcagggtgctggagctgtatagccagaaggaactgcgctccattaacgtgtttgatctggaggaaatcatgagggagttcaattcctacaagcagcgcgtgaaatatgtcgaagataaggtggtcgaccctctgccaccctacctgaaaggcctggacgatctgatgagcgggctgggagctgcaggcaaggcagtgggagtcgccatcggagctgtgggaggcgctgtcgcatccgtggtcgagggagtggctacctttctgaagaacccattctttttcatcatcggcctgatcattgggctgttcctggtgctgagagtcggcatccacctgtgcattaagctgaagcacaccaagaagaggcagatctacaccgatattgaaatgaacagactgggcaagtga

SEQ ID NO: 25 - RNA, 720 nt, synthetic construct
  source 1..720: mol_type = other RNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 26 - protein, 1255 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 27 - DNA, 3768 bp, synthetic construct
  source 1..3768: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtagtgcaagtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatttttctttatcatagggttaatcattggactattcttggttctccgagttggtatccatctttgcattaaattaaagcacaccaagaaaagacagatttatacagacatagagatgaaccgacttggaaagtaa

SEQ ID NO: 28 - DNA, 3768 bp, synthetic construct
  source 1..3768: mol_type = other DNA
atgttcgtgttcctggtgctgctgcctctggtgagctcccagtgcgtgaacctgaccacaaggacccagctcccccctgcctataccaattcctttacaaggggcgtgtactatccagacaaggtgttccgctctagcgtgctgcactctacacaggatctgttcctgcccttctttagcaacgtgacctggtttcacgccatccatgtgagcggcaccaatggcacaaagcggtttgacaatcctgtgctgccattcaacgatggcgtgtactttgcctccaccgagaagtctaacatcatcagaggctggatcttcggcaccacactggacagcaagacacagtccctgctgatcgtgaacaatgccaccaacgtggtcatcaaggtgtgcgagtttcagttctgtaatgatccttttctgggcgtgtactatcacaagaacaataagtcttggatggagagcgagttccgcgtgtattcctctgccaacaattgtacattcgagtacgtgtcccagccatttctgatggacctggagggcaagcagggcaacttcaagaacctgcgggagttcgtgttcaagaatatcgatggctatttcaagatctactctaagcacacccctatcaacctggtgcgcgacctgccacagggctttagcgccctggagcctctggtggatctgccaatcggcatcaacatcaccaggttccagacactgctggccctgcaccgcagctacctgacacctggcgacagctcctctggatggaccgccggggccgccgcctactatgtgggctatctgcagccacggacctttctgctgaagtacaacgagaatggcaccatcacagacgcagtggattgcgccctggaccccctgagcgagaccaagtgtacactgaagtccttcaccgtggagaagggcatctatcagacatccaattttcgggtgcagcccaccgagtctatcgtgagattcccaaatatcacaaacctgtgccccttcggcgaggtgtttaacgccaccagattcgccagcgtgtacgcctggaatcggaagagaatctctaactgcgtggccgactatagcgtgctgtacaactctgccagcttttccaccttcaagtgctatggcgtgtcccccacaaagctgaatgacctgtgcttcaccaacgtgtacgccgattcttttgtgatcaggggcgacgaggtgagacagatcgcaccaggccagacaggcaagatcgccgactacaattataagctgcccgacgatttcaccggctgcgtgatcgcctggaactctaacaatctggatagcaaagtgggcggcaactacaattatctgtacaggctgttccgcaagagcaatctgaagccttttgagcgggacatctctacagagatctaccaggccggcagcaccccatgcaatggcgtggagggcttcaactgttattttcccctgcagtcctacggctttcagcctaccaacggcgtgggctatcagccatacagagtggtggtgctgagcttcgagctgctgcacgcaccagcaacagtgtgcggacctaagaagtccaccaatctggtgaagaacaagtgcgtgaacttcaacttcaacggcctgaccggcacaggcgtgctgaccgagtccaataagaagtttctgcccttccagcagtttggccgggacatcgccgataccacagacgccgtgagagacccccagaccctggagatcctggacatcacaccttgctccttcggcggcgtgtctgtgatcacacctggcaccaatacaagcaaccaggtggccgtgctgtatcaggacgtgaattgtaccgaggtgccagtggccatccacgccgatcagctcacccccacatggcgggtgtactccaccggctctaacgtgttccagacaagagccggctgcctgatcggagccgagcatgtgaacaattcctatgagtgcgacatccccatcggagccggcatctgtgcctcttaccagacccagacaaacagccctggctccgccagctccgtggcctctcagagcatcatcgcctataccatgagcctgggggccgagaacagcgtggcctactctaacaatagcatcgccatccccaccaactttacaatctctgtgaccacagagatcctgcctgtgagcatgaccaagacatccgtggactgcacaatgtatatctgtggcgattccaccgagtgctctaacctgctgctgcagtacggcagcttctgtacccagctcaaccgggccctgacaggaatcgcagtggagcaggacaagaacacacaggaggtgtttgcccaggtgaagcagatctacaagaccccacccatcaaggacttcggcggctttaatttctcccagatcctgccagatccctccaagccatctaagcggagcttcatcgaggacctgctgtttaacaaggtgaccctggccgatgccggctttatcaagcagtatggcgattgcctgggcgacatcgccgccagagacctgatctgtgcccagaagttcaatggcctgaccgtgctgcctccactgctgacagatgagatgatcgcacagtacacaagcgccctgctggccggcaccatcacatccggatggaccttcggggccggggccgccctgcagatccccttcgccatgcagatggcctataggtttaacggcatcggcgtgacccagaatgtgctgtacgagaaccagaagctgatcgccaatcagttcaactccgccatcggcaagatccaggacagcctgtctagcacagcctccgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgacaggctgatcacaggccgcctgcagtctctgcagacctatgtgacacagcagctcatcagggccgccgagatcagagccagcgccaatctggccgccaccaagatgtccgagtgcgtgctgggccagtctaagcgcgtggacttctgtggcaagggctatcacctgatgagctttccacagtccgccccccacggagtggtgttcctgcatgtgacctacgtgcctgcccaggagaagaactttaccacagccccagccatctgccacgatggcaaggcacacttccccagggagggcgtgttcgtgagcaacggcacccactggttcgtgacacagcgcaacttctacgagcctcagatcatcaccacagacaatacattcgtgtctggcaactgtgacgtggtcatcggcatcgtgaacaataccgtgtatgatcccctgcagcctgagctggacagcttcaaggaggagctggataagtactttaagaatcacacctcccccgacgtggatctgggcgacatctctggcatcaatgccagcgtggtgaacatccagaaggagatcgacaggctgaacgaggtggccaagaatctgaacgagagcctgatcgatctgcaggagctgggcaagtatgagcagtacatcaagttctttttcatcatcggcctgatcatcggcctgttcctggtgctgcgcgtgggcatccacctgtgcatcaagctgaagcacaccaagaagaggcagatctacacagacatcgagatgaaccgcctgggcaagtga

SEQ ID NO: 29 - RNA, 3768 nt, synthetic construct
  source 1..3768: mol_type = other RNA
atgttcgtgttcctggtgctgctgcctctggtgagctcccagtgcgtgaacctgaccacaaggacccagctcccccctgcctataccaattcctttacaaggggcgtgtactatccagacaaggtgttccgctctagcgtgctgcactctacacaggatctgttcctgcccttctttagcaacgtgacctggtttcacgccatccatgtgagcggcaccaatggcacaaagcggtttgacaatcctgtgctgccattcaacgatggcgtgtactttgcctccaccgagaagtctaacatcatcagaggctggatcttcggcaccacactggacagcaagacacagtccctgctgatcgtgaacaatgccaccaacgtggtcatcaaggtgtgcgagtttcagttctgtaatgatccttttctgggcgtgtactatcacaagaacaataagtcttggatggagagcgagttccgcgtgtattcctctgccaacaattgtacattcgagtacgtgtcccagccatttctgatggacctggagggcaagcagggcaacttcaagaacctgcgggagttcgtgttcaagaatatcgatggctatttcaagatctactctaagcacacccctatcaacctggtgcgcgacctgccacagggctttagcgccctggagcctctggtggatctgccaatcggcatcaacatcaccaggttccagacactgctggccctgcaccgcagctacctgacacctggcgacagctcctctggatggaccgccggggccgccgcctactatgtgggctatctgcagccacggacctttctgctgaagtacaacgagaatggcaccatcacagacgcagtggattgcgccctggaccccctgagcgagaccaagtgtacactgaagtccttcaccgtggagaagggcatctatcagacatccaattttcgggtgcagcccaccgagtctatcgtgagattcccaaatatcacaaacctgtgccccttcggcgaggtgtttaacgccaccagattcgccagcgtgtacgcctggaatcggaagagaatctctaactgcgtggccgactatagcgtgctgtacaactctgccagcttttccaccttcaagtgctatggcgtgtcccccacaaagctgaatgacctgtgcttcaccaacgtgtacgccgattcttttgtgatcaggggcgacgaggtgagacagatcgcaccaggccagacaggcaagatcgccgactacaattataagctgcccgacgatttcaccggctgcgtgatcgcctggaactctaacaatctggatagcaaagtgggcggcaactacaattatctgtacaggctgttccgcaagagcaatctgaagccttttgagcgggacatctctacagagatctaccaggccggcagcaccccatgcaatggcgtggagggcttcaactgttattttcccctgcagtcctacggctttcagcctaccaacggcgtgggctatcagccatacagagtggtggtgctgagcttcgagctgctgcacgcaccagcaacagtgtgcggacctaagaagtccaccaatctggtgaagaacaagtgcgtgaacttcaacttcaacggcctgaccggcacaggcgtgctgaccgagtccaataagaagtttctgcccttccagcagtttggccgggacatcgccgataccacagacgccgtgagagacccccagaccctggagatcctggacatcacaccttgctccttcggcggcgtgtctgtgatcacacctggcaccaatacaagcaaccaggtggccgtgctgtatcaggacgtgaattgtaccgaggtgccagtggccatccacgccgatcagctcacccccacatggcgggtgtactccaccggctctaacgtgttccagacaagagccggctgcctgatcggagccgagcatgtgaacaattcctatgagtgcgacatccccatcggagccggcatctgtgcctcttaccagacccagacaaacagccctggctccgccagctccgtggcctctcagagcatcatcgcctataccatgagcctgggggccgagaacagcgtggcctactctaacaatagcatcgccatccccaccaactttacaatctctgtgaccacagagatcctgcctgtgagcatgaccaagacatccgtggactgcacaatgtatatctgtggcgattccaccgagtgctctaacctgctgctgcagtacggcagcttctgtacccagctcaaccgggccctgacaggaatcgcagtggagcaggacaagaacacacaggaggtgtttgcccaggtgaagcagatctacaagaccccacccatcaaggacttcggcggctttaatttctcccagatcctgccagatccctccaagccatctaagcggagcttcatcgaggacctgctgtttaacaaggtgaccctggccgatgccggctttatcaagcagtatggcgattgcctgggcgacatcgccgccagagacctgatctgtgcccagaagttcaatggcctgaccgtgctgcctccactgctgacagatgagatgatcgcacagtacacaagcgccctgctggccggcaccatcacatccggatggaccttcggggccggggccgccctgcagatccccttcgccatgcagatggcctataggtttaacggcatcggcgtgacccagaatgtgctgtacgagaaccagaagctgatcgccaatcagttcaactccgccatcggcaagatccaggacagcctgtctagcacagcctccgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgacaggctgatcacaggccgcctgcagtctctgcagacctatgtgacacagcagctcatcagggccgccgagatcagagccagcgccaatctggccgccaccaagatgtccgagtgcgtgctgggccagtctaagcgcgtggacttctgtggcaagggctatcacctgatgagctttccacagtccgccccccacggagtggtgttcctgcatgtgacctacgtgcctgcccaggagaagaactttaccacagccccagccatctgccacgatggcaaggcacacttccccagggagggcgtgttcgtgagcaacggcacccactggttcgtgacacagcgcaacttctacgagcctcagatcatcaccacagacaatacattcgtgtctggcaactgtgacgtggtcatcggcatcgtgaacaataccgtgtatgatcccctgcagcctgagctggacagcttcaaggaggagctggataagtactttaagaatcacacctcccccgacgtggatctgggcgacatctctggcatcaatgccagcgtggtgaacatccagaaggagatcgacaggctgaacgaggtggccaagaatctgaacgagagcctgatcgatctgcaggagctgggcaagtatgagcagtacatcaagttctttttcatcatcggcctgatcatcggcctgttcctggtgctgcgcgtgggcatccacctgtgcatcaagctgaagcacaccaagaagaggcagatctacacagacatcgagatgaaccgcctgggcaagtga

SEQ ID NO: 30 - protein, 1255 aa, synthetic construct
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 31 - DNA, 3768 bp, synthetic construct
  source 1..3768: mol_type = other DNA
atgttcatctttctgctgttcctgaccctgacaagcggctccgacctggataggtgcaccacatttgacgatgtgcaggcccccaactacacacagcacaccagctccatgaggggcgtgtactatcctgatgagatcttccgctctgacacactgtacctgacccaggacctgttcctgcctttttatagcaatgtgacaggcttccacaccatcaatcacacatttggcaaccccgtgatccctttcaaggatggcatctactttgccgccaccgagaagtctaacgtggtgcggggatgggtgttcggcagcacaatgaacaataagtctcagagcgtgatcatcatcaacaatagcaccaacgtggtcatcagagcctgcaattttgagctgtgcgacaaccccttctttgccgtgtccaagcctatgggcacccagacacacaccatgatctttgataatgccttcaactgtacctttgagtacatcagcgatgccttttccctggacgtgtctgagaagtccggcaacttcaagcacctgagggagttcgtgtttaagaataaggacggcttcctgtacgtgtataagggctatcagcccatcgatgtggtgcgcgacctgccttccggcttcaacaccctgaagccaatctttaagctgcccctgggcatcaatatcaccaacttcagggccatcctgacagcctttagcccagcacaggacatctggggcaccagcgccgccgcctacttcgtgggctatctgaagcccaccaccttcatgctgaagtacgatgagaacggcacaatcaccgacgccgtggattgcagccagaatccactggccgagctgaagtgttccgtgaagtctttcgagatcgacaagggcatctatcagacctccaactttagggtggtgccatctggcgatgtggtgcgcttcccaaatatcaccaacctgtgccccttcggcgaggtgtttaatgccacaaagttccccagcgtgtacgcctgggagcgcaagaagatcagcaactgcgtggccgactactccgtgctgtataatagcaccttcttcagcaccttcaagtgctacggcgtgagcgccaccaagctgaatgacctgtgcttctctaacgtgtatgccgatagctttgtggtgaagggcgacgatgtgaggcagatcgcacctggacagaccggcgtgatcgcagactacaactataagctgccagacgatttcatgggctgcgtgctggcctggaatacacgcaacatcgatgccacatccaccggcaactacaattataagtaccggtatctgagacacggcaagctgcggcccttcgagagagacatctccaatgtgccattttctccagatggcaagccatgcaccccacctgccctgaattgttactggcctctgaacgactacggcttctataccacaaccggcatcggctaccagccttatagggtggtggtgctgtcctttgagctgctgaacgcacctgcaaccgtgtgcggaccaaagctgtctacagatctgatcaagaatcagtgcgtgaacttcaacttcaacggcctgacaggcaccggcgtgctgaccccttctagcaagcggttccagccatttcagcagttcggcagagacgtgagcgatttcaccgactccgtgcgcgacccaaagacatccgagatcctggacatcagcccctgctcctttggcggcgtgtctgtgatcacacctggcaccaacgcctcctctgaggtggccgtgctgtaccaggatgtgaattgtaccgacgtgagcacagcaatccacgcagaccagctcaccccagcatggcggatctattccaccggcaacaacgtgttccagacacaggcaggatgcctgatcggagccgagcatgtggatacaagctacgagtgcgacatccccatcggagccggcatctgtgcctcttatcacaccgtgagcctgctgagatccacatctcagaagtctatcgtggcctacaccatgagcctgggggccgatagctccatcgcctattccaacaataccatcgccatcccaacaaacttcagcatctccatcacaaccgaagtgatgcccgtgtccatggccaagacctctgtggactgcaacatgtacatctgtggcgatagcacagagtgcgccaatctgctgctgcagtatggctccttttgtacccagctcaaccgggccctgtctggaatcgccgccgagcaggacaggaatacacgcgaggtgttcgcccaggtgaagcagatgtacaagacacctaccctgaagtattttggcggcttcaacttttctcagatcctgcctgatccactgaagccaaccaagcggagcttcatcgaggacctgctgtttaataaggtgacactggccgatgccggcttcatgaagcagtacggcgagtgcctgggcgacatcaacgccagagacctgatctgtgcccagaagtttaatggcctgaccgtgctgccacccctgctgacagacgatatgatcgcagcatataccgccgccctggtgtccggcacagccaccgccggctggaccttcggggccggggccgccctgcagatccctttcgccatgcagatggcctaccggtttaacggcatcggcgtgacccagaatgtgctgtatgagaaccagaagcagatcgccaatcagtttaacaaggccatcagccagatccaggagtccctgacaaccacatctaccgccctgggcaagctgcaggacgtggtgaatcagaacgcccaggccctgaatacactggtgaagcagctcagcagcaacttcggggccatcagcagcgtgctgaacgacatcctgagccggctggacccgccggaggcagaggtgcagatcgataggctgatcaccggcagactgcagtctctgcagacatacgtgacccagcagctcatcagggccgccgagatcagagccagcgccaacctggccgccacaaagatgtccgagtgcgtgctgggccagtctaagagggtggacttctgtggcaagggctaccacctgatgtcctttccacaggccgcccctcacggagtggtgttcctgcatgtgacctatgtgccttctcaggagcgcaactttaccacagccccagcaatctgccacgagggcaaggcatacttcccccgggagggcgtgttcgtgtttaacggcacctcctggtttatcacacagagaaatttcttttcccctcagatcatcaccacagacaataccttcgtgagcggcaactgtgacgtggtcatcggcatcatcaacaatacagtgtacgatcctctgcagccagagctggacagcttcaaggaggagctggataagtacttcaagaaccacacctcccccgacgtggatctgggcgacatcagcggcatcaatgcctccgtggtgaacatccagaaggagatcgacagactgaatgaggtggccaagaatctgaacgagtccctgatcgatctgcaggagctgggcaagtacgagcagtatatcaagtggccatggtacgtgtggctgggcttcatcgccggcctgatcgccatcgtgatggtgaccatcctgctgtgctgtatgacatcttgctgtagctgcctgaagggagcctgctcctgtggctcttgctgtaagtttgacgaggacgatagcgagcccgtgctgaagggcgtgaagctgcactatacctga

SEQ ID NO: 32 - DNA, 3768 bp, synthetic construct
  source 1..3768: mol_type = other DNA
atgttcatcttcctgctgttcctgactctgaccagcggatctgatctggaccggtgcaccacctttgacgacgtgcaggcccctaactacacccagcacacctccagcatgagaggcgtgtactaccccgacgagattttcagatcagacaccctgtacctcacccaggacctgttcctgcctttctacagcaacgtgaccggctttcacaccatcaatcacaccttcggcaaccctgtgatacccttcaaggatgggatctactttgccgccacagagaaaagcaacgtggtccggggctgggtgtttgggtctacaatgaacaacaagagccagagcgtgatcatcatcaacaatagcaccaacgtggtcatcagagcttgtaatttcgagctgtgcgacaacccattcttcgccgtgtccaagcctatgggcacccagacccacacgatgatcttcgataatgccttcaattgcaccttcgagtacatctccgatgctttttctctcgacgtctctgagaagtccggcaatttcaagcacctgagagagttcgtgttcaaaaacaaagacggcttcctgtacgtgtacaagggctaccagccaatcgacgtggtgcgggacctgcctagcggctttaacacactgaagcccatcttcaagctgcctctgggcatcaacatcacaaactttagagccatcctgaccgcttttagccctgcccaggacatctggggcaccagcgccgctgcatacttcgtgggctacctgaaacccaccacatttatgctgaagtacgacgagaacggcacaatcaccgacgccgtggactgctctcagaaccccctggccgagctgaaatgttccgtgaagagctttgaaatcgataagggcatctaccagaccagcaactttcgggtggtgccttctggagacgtggtgcgcttccctaacattacaaacctgtgtccattcggcgaagtgtttaacgccaccaagttcccttctgtgtacgcctgggagagaaagaaaatcagcaactgcgtggccgattacagcgtgctgtataacagtacatttttcagcacattcaagtgttacggcgttagcgccacaaagcttaatgacttgtgcttctcaaacgtgtacgccgacagcttcgtggtcaagggcgacgacgtgcggcagattgcccctggtcagaccggcgtgatagccgattacaactacaagctgcccgacgatttcatgggctgcgtgctggcctggaacaccagaaatatcgacgccacaagcaccggcaattacaactacaagtatagatacctgaggcacggcaagctgagacctttcgagagagacatcagcaacgtgccattcagccctgatggcaagccttgcaccccacctgccctcaactgctactggcccctgaacgactacggcttctacaccacaacaggcattggctatcagccttacagagttgtggttctgagcttcgaactgctgaacgctcctgctaccgtttgtggccctaagctgagcaccgacctgatcaagaaccagtgcgtgaacttcaacttcaatggccttaccggcacgggcgtgctgacccctagcagcaaaagattccagccttttcagcagttcggcagagacgtgtcagacttcaccgatagcgtgcgggaccccaagacctctgaaatcctggacatcagcccatgttctttcggcggagtgagcgtgatcacccctggcactaacgcctcctccgaggtggccgtgctgtaccaggacgttaattgcaccgacgtgtccacagccatccacgccgaccaactgacccctgcttggagaatctactctaccggcaacaacgttttccagacacaggctggatgtctgatcggagccgagcatgtggacaccagctacgagtgcgacatccccatcggagccgggatctgcgccagctaccacaccgtgtccctgctccggtctaccagccagaagagcatagtggcttacacaatgagcctgggagccgacagcagcatcgcttatagcaacaacaccatcgccatccctaccaacttctctatctccatcacaactgaagtcatgcctgtcagcatggccaagaccagcgtggactgcaacatgtacatctgcggcgattctaccgaatgcgccaacctgctgctccagtacgggtccttctgcacccaactgaacagggccctgtccggcatcgccgctgaacaggaccggaacacaagagaagtgttcgcccaagtgaagcaaatgtacaagacccccaccctgaagtacttcggcggcttcaacttcagccagatcctgcctgaccctctgaaacctacaaaaagaagcttcatcgaggacctgctgtttaacaaggtgaccctggccgacgccggcttcatgaaacagtacggcgagtgtctgggagacatcaacgccagagacctgatctgcgcccaaaaattcaatggactcacagtgctgccccccctgctgactgatgatatgatcgccgcctacactgctgctctggtgtccggcaccgccacagccggctggacctttggagccggagccgccctgcagatcccctttgccatgcagatggcctatcggttcaacgggatcggagttacacagaacgttctgtacgagaaccagaagcaaatcgccaatcagttcaacaaggccattagccagatccaggagagcctgacaaccaccagcaccgccctgggcaagctgcaagacgtagtgaatcagaacgcccaggccctgaataccctggtgaagcaactgtcaagcaatttcggcgctatcagcagcgtgctgaacgacatcctgagcagactggaccctcccgaagccgaggttcagatcgaccggctgatcacagggagactgcagtccctgcaaacatacgtgacccagcaactgatccgggccgccgaaatcagagctagtgccaacctggccgccaccaagatgagcgagtgcgtgctgggacaatctaagagagtcgacttctgcggcaagggctatcacctgatgagcttcccccaggccgctcctcatggcgtggtcttcctgcatgtgacctacgtgccctcccaggaacggaacttcaccacagcccctgccatctgccacgagggaaaagcctactttcccagagagggcgtgttcgtgtttaacggcacatcttggttcatcacacagagaaacttcttcagccctcagatcattacaacagataacaccttcgtcagcggaaattgtgacgtggtgattggcatcatcaacaacaccgtgtacgaccctctgcagcccgaactggacagcttcaaggaggaactggacaagtactttaagaaccacacatctccagatgtggacctgggcgacatctccggaatcaacgccagcgtggtgaacatccagaaagaaatcgacagactgaatgaggttgctaagaacctgaacgagtctttgatcgatctgcaggagctggggaagtacgagcagtacatcaagtggccatggtacgtgtggctgggcttcatcgccggcctgatcgccattgtgatggtgacaatcctgctgtgctgcatgacaagctgctgtagctgcctgaagggcgcttgttcctgtggctcttgctgcaagttcgatgaagatgatagcgaacccgtgctgaagggcgtgaagctgcactacacctga

SEQ ID NO: 33 - RNA, 3768 nt, synthetic construct
  source 1..3768: mol_type = other RNA
atgttcatcttcctgctgttcctgactctgaccagcggatctgatctggaccggtgcaccacctttgacgacgtgcaggcccctaactacacccagcacacctccagcatgagaggcgtgtactaccccgacgagattttcagatcagacaccctgtacctcacccaggacctgttcctgcctttctacagcaacgtgaccggctttcacaccatcaatcacaccttcggcaaccctgtgatacccttcaaggatgggatctactttgccgccacagagaaaagcaacgtggtccggggctgggtgtttgggtctacaatgaacaacaagagccagagcgtgatcatcatcaacaatagcaccaacgtggtcatcagagcttgtaatttcgagctgtgcgacaacccattcttcgccgtgtccaagcctatgggcacccagacccacacgatgatcttcgataatgccttcaattgcaccttcgagtacatctccgatgctttttctctcgacgtctctgagaagtccggcaatttcaagcacctgagagagttcgtgttcaaaaacaaagacggcttcctgtacgtgtacaagggctaccagccaatcgacgtggtgcgggacctgcctagcggctttaacacactgaagcccatcttcaagctgcctctgggcatcaacatcacaaactttagagccatcctgaccgcttttagccctgcccaggacatctggggcaccagcgccgctgcatacttcgtgggctacctgaaacccaccacatttatgctgaagtacgacgagaacggcacaatcaccgacgccgtggactgctctcagaaccccctggccgagctgaaatgttccgtgaagagctttgaaatcgataagggcatctaccagaccagcaactttcgggtggtgccttctggagacgtggtgcgcttccctaacattacaaacctgtgtccattcggcgaagtgtttaacgccaccaagttcccttctgtgtacgcctgggagagaaagaaaatcagcaactgcgtggccgattacagcgtgctgtataacagtacatttttcagcacattcaagtgttacggcgttagcgccacaaagcttaatgacttgtgcttctcaaacgtgtacgccgacagcttcgtggtcaagggcgacgacgtgcggcagattgcccctggtcagaccggcgtgatagccgattacaactacaagctgcccgacgatttcatgggctgcgtgctggcctggaacaccagaaatatcgacgccacaagcaccggcaattacaactacaagtatagatacctgaggcacggcaagctgagacctttcgagagagacatcagcaacgtgccattcagccctgatggcaagccttgcaccccacctgccctcaactgctactggcccctgaacgactacggcttctacaccacaacaggcattggctatcagccttacagagttgtggttctgagcttcgaactgctgaacgctcctgctaccgtttgtggccctaagctgagcaccgacctgatcaagaaccagtgcgtgaacttcaacttcaatggccttaccggcacgggcgtgctgacccctagcagcaaaagattccagccttttcagcagttcggcagagacgtgtcagacttcaccgatagcgtgcgggaccccaagacctctgaaatcctggacatcagcccatgttctttcggcggagtgagcgtgatcacccctggcactaacgcctcctccgaggtggccgtgctgtaccaggacgttaattgcaccgacgtgtccacagccatccacgccgaccaactgacccctgcttggagaatctactctaccggcaacaacgttttccagacacaggctggatgtctgatcggagccgagcatgtggacaccagctacgagtgcgacatccccatcggagccgggatctgcgccagctaccacaccgtgtccctgctccggtctaccagccagaagagcatagtggcttacacaatgagcctgggagccgacagcagcatcgcttatagcaacaacaccatcgccatccctaccaacttctctatctccatcacaactgaagtcatgcctgtcagcatggccaagaccagcgtggactgcaacatgtacatctgcggcgattctaccgaatgcgccaacctgctgctccagtacgggtccttctgcacccaactgaacagggccctgtccggcatcgccgctgaacaggaccggaacacaagagaagtgttcgcccaagtgaagcaaatgtacaagacccccaccctgaagtacttcggcggcttcaacttcagccagatcctgcctgaccctctgaaacctacaaaaagaagcttcatcgaggacctgctgtttaacaaggtgaccctggccgacgccggcttcatgaaacagtacggcgagtgtctgggagacatcaacgccagagacctgatctgcgcccaaaaattcaatggactcacagtgctgccccccctgctgactgatgatatgatcgccgcctacactgctgctctggtgtccggcaccgccacagccggctggacctttggagccggagccgccctgcagatcccctttgccatgcagatggcctatcggttcaacgggatcggagttacacagaacgttctgtacgagaaccagaagcaaatcgccaatcagttcaacaaggccattagccagatccaggagagcctgacaaccaccagcaccgccctgggcaagctgcaagacgtagtgaatcagaacgcccaggccctgaataccctggtgaagcaactgtcaagcaatttcggcgctatcagcagcgtgctgaacgacatcctgagcagactggaccctcccgaagccgaggttcagatcgaccggctgatcacagggagactgcagtccctgcaaacatacgtgacccagcaactgatccgggccgccgaaatcagagctagtgccaacctggccgccaccaagatgagcgagtgcgtgctgggacaatctaagagagtcgacttctgcggcaagggctatcacctgatgagcttcccccaggccgctcctcatggcgtggtcttcctgcatgtgacctacgtgccctcccaggaacggaacttcaccacagcccctgccatctgccacgagggaaaagcctactttcccagagagggcgtgttcgtgtttaacggcacatcttggttcatcacacagagaaacttcttcagccctcagatcattacaacagataacaccttcgtcagcggaaattgtgacgtggtgattggcatcatcaacaacaccgtgtacgaccctctgcagcccgaactggacagcttcaaggaggaactggacaagtactttaagaaccacacatctccagatgtggacctgggcgacatctccggaatcaacgccagcgtggtgaacatccagaaagaaatcgacagactgaatgaggttgctaagaacctgaacgagtctttgatcgatctgcaggagctggggaagtacgagcagtacatcaagtggccatggtacgtgtggctgggcttcatcgccggcctgatcgccattgtgatggtgacaatcctgctgtgctgcatgacaagctgctgtagctgcctgaagggcgcttgttcctgtggctcttgctgcaagttcgatgaagatgatagcgaacccgtgctgaagggcgtgaagctgcactacacctga

SEQ ID NO: 34 - protein, 30 aa, synthetic construct
WEARLARALARALARHLARALARALRACEA